(19) United States
(12) Patent Application Publication
(10) Pub. No.: US 2024/0376220 A1
(43) Pub. Date: Nov. 14, 2024

(54) Multivalent and Multispecific DR5-Binding Fusion Proteins

(71) Applicant: Inhibrx Biosciences, Inc., La Jolla, CA (US)

(72) Inventors: John C. Timmer, San Diego, CA (US); Kyle S. Jones, San Marcos, CA (US); Amir S. Razai, La Jolla, CA (US); Abrahim Hussain, La Jolla, CA (US); Katelyn M. Willis, San Diego, CA (US); Quinn Deveraux, La Jolla, CA (US); Brendan P. Eckelman, Encinitas, CA (US)

(73) Assignee: Inhibrx Biosciences, Inc., La Jolla, CA (US)

(21) Appl. No.: 18/626,699

(22) Filed: Apr. 4, 2024

Related U.S. Application Data
(62) Division of application No. 17/394,900, filed on Aug. 5, 2021, now Pat. No. 11,976,126.
Application No. 17/394,900 is a division of application No. 16/387,754, filed on Apr. 18, 2019, now Pat. No. 11,117,973.
Application No. 16/387,754 is a division of application No. 15/213,296, filed on Jul. 18, 2016, now Pat. No. 10,308,720.
(60) Provisional application No. 62/193,309, filed on Jul. 16, 2015.

(51) Int. Cl. C07K 16/28; A61K 39/00; A61K 39/395

(52) U.S. Cl. CPC C07K 16/2878; A61K 39/39558; A61K 2039/505; C07K 2317/22; C07K 2317/24; C07K 2317/33; C07K 2317/35; C07K 2317/567; C07K 2317/569; C07K 2317/73; C07K 2317/75

(57) ABSTRACT

The disclosure relates generally to molecules that specifically engage death receptor 5 (DR5), a member of the TNF receptor superfamily (TNFRSF). More specifically the disclosure relates to multivalent and multispecific molecules that bind at least DR5.

### RELATED APPLICATIONS

This application is a divisional of U.S. patent application Ser. No. 17/394,900, filed Aug. 5, 2021, which is a divisional of U.S. patent application Ser. No. 16/387,754, filed Apr. 18, 2019, now U.S. Pat. No. 11,117,973, which is a divisional of U.S. patent application Ser. No. 15/213,296, filed Jul. 18, 2016, now U.S. Pat. No. 10,308,720, which claims the benefit of U.S. Provisional Application No. 62/193,309, filed Jul. 16, 2015, the contents of which are incorporated herein by reference in their entirety.

### FIELD OF THE INVENTION

The disclosure relates generally to molecules that specifically engage death receptor 5 (DR5), a member of the TNF receptor superfamily (TNFRSF). More specifically the disclosure relates to multivalent and multispecific molecules that bind at least DR5.

### BACKGROUND OF THE INVENTION

The tumor necrosis factor receptor superfamily consists of several structurally related cell surface receptors. Activation by multimeric ligands is common feature of many of these receptors. Many members of the TNFRSF have therapeutic utility in numerous pathologies, if activated properly. Importantly to properly agonize this receptor family often requires higher order clustering and conventional bivalent antibodies are not ideal for this. Therefore, there exists a therapeutic need for more potent agonist molecules of the TNFRSF.

### SUMMARY OF THE INVENTION

The disclosure provides multivalent fusion polypeptides that bind at least death receptor 5 (DR5, also known as TRAIL receptor 2 (TRAILR2), or tumor necrosis factor receptor superfamily member 10B (TNFRSF10B)). These DR5 binding fusion polypeptides are also referred to herein as DR5-targeting molecules. DR5 is a member of the TNF receptor superfamily (TNFRSF) and a cell surface receptor of the TNF-receptor superfamily that binds TNF-related apoptosis-inducing ligand (TRAIL). TRAIL evolved to play critical roles in mammalian development and host defense by selectively eradicating unwanted, infected and malignant cells from healthy cell populations. On binding the TNF receptor family members DR4 or DR5, TRAIL induces cell death via caspase-dependent apoptosis. DR5 appears to be the primary receptor on tumor cells that facilitates the observed tumor biased activity of the TRAIL pathway. DR5 is activated by the natural ligand TRAIL, which brings three DR5 receptors within close proximity thereby activating intracellular caspase-8 and initiating activation of other death-inducing caspases, such as caspases-9 and caspases-3. Thus initiation of this cell death pathway requires clustering of DR5 receptors for efficient cell death.

Conventional antibodies targeting members of the TNF receptor superfamily (TNFRSF) have been shown to require an exogenous crosslinking to achieve sufficient agonist activity, as evidenced by the necessity for Fc-gamma Receptor (FcγRs) for the activity antibodies to DR4, DR5, GITR and OX40 (Ichikawa et al 2001 al Nat. Med. 7, 954-960, Li et al 2008 Drug Dev. Res. 69, 69-82; Pukac et al 2005 Br. J. Cancer 92, 1430-1441; Yanda et al 2008 Ann. Oncol. 19, 1060-1067; Yang et al 2007 Cancer Lett. 251:146-157; Bulliard et al 2013 JEM 210(9): 1685; Bulliard et al 2014 Immunol and Cell Biol 92: 475-480). In addition to crosslinking via FcγRs, other exogenous agents including addition of the oligomeric ligand or antibody binding entities (e.g. protein A and secondary antibodies) have be demonstrated to enhance anti-TNFRSF antibody clustering and downstream signaling. For instance, in vitro agonist activity of the CD137 antibody, PF-05082566, requires crosslinking via a secondary antibody (Fisher et al Cancer Immunol Immunother 2012 61:1721-1733). These findings suggest the need for clustering of TNFRSFs beyond a dimer.

Efforts to clinically exploit the TRAIL pathway for cancer therapy relied upon a recombinant version of the natural ligand TRAIL and antibodies specific for DR5. Antibody agonists targeting DR5 required a crosslinking agent in preclinical in vitro experiments. For example, the addition of the DR5 ligand TRAIL enhanced the apoptosis inducing ability of an anti-DR5 antibody, AMG655 (Graves et al2014 Cancer Cell 26: 177-189). Conventional antibodies are bivalent and capable clustering only two DR5 receptors (one per each FAB arm). Consistent with other members of the TNFRSF, clustering of two DR5 receptors is insufficient to mediate signaling and activate the cell death pathway in vitro. Surprisingly in vivo administration of DR5 targeting antibodies in pre-clinical mouse models of human cancers showed significant activity in a wide variety of tumor types. This activity was later shown to be dependent on mouse FcgammaR (FcγR) receptors. Clinical studies in humans failed to reproduce the robust responses seen in these pre-clinical mouse models. The lack of activity in humans is hypothesized to be due to insufficient antibody crosslinking. This may be due to differences in serum IgG, FcγR and or TRAIL concentrations between immune compromised mice and human cancer patients.

The present disclosure provides multivalent fusion proteins targeting DR5 that are capable of potently agonizing DR5 signaling mediating direct cell death. The fusion proteins of the present disclosure can be bivalent, trivalent, tetravalent, pentavalent, or hexavalent. Importantly, the fusion proteins of the present disclosure are capable of eliciting apoptosis of DR5 expressing cells independently of exogenous crosslinking agents.

In some embodiments, the fusion proteins of the present disclosure incorporate a binding domain (DR5BD) that binds DR5. In preferred embodiments, the DR5 binding DR5BD does not bind DR4, decoy R1, decoy R2, Osteopontin, or any other TNFRSF member. In preferred embodiments the DR5 binding DR5BD binds human and cynomolgus monkey DR5. In some embodiments, the DR5 binding DR5BD blocks the interaction of DR5 and its ligand TRAIL. In other embodiments, the DR5 binding DR5BD does not block the interaction of DR5 and its ligand TRAIL. In some embodiments, the fusion protein of the present disclosure incorporates multiple DR5 binding DR5BDs that recognize distinct epitopes on DR5. In some embodiments, the fusion protein of the present disclosure incorporates multiple DR5 binding DR5BDs, wherein some DR5BDs block the DR5-TRAIL interaction and other do not block the DR5-TRAIL interaction. In preferred embodiments, DR5 targeting fusion proteins of the present disclosure induce direct cell death of tumor cells. The DR5 targeting fusion proteins of the present disclosure have utility in treating tumors both hematologic and solid in nature.

The present disclosure provides multivalent DR5 binding fusion proteins, which comprise 2 or more DR5 binding domains (DR5BDs). In some embodiments, the fusion proteins of the present disclosure have utility in treating neoplasms. In some embodiments, the fusion proteins of the present disclosure bind DR5 expressed on a tumor cell. In some embodiments, the fusion protein contains two or more different DR5BDs, where each DR5BD binds DR5. In some embodiments, the fusion protein contains multiple copies of a DR5BD that binds DR5. For example, in some embodiments, the fusion protein contains at least two copies of a DR5BD that binds DR5. In some embodiments, the fusion protein contains at least three copies of a DR5BD that binds DR5. In some embodiments, the fusion protein contains at least four copies of a DR5BD that binds DR5. In some embodiments, the fusion protein contains at least five copies of a DR5BD that binds DR5. In some embodiments, the fusion protein contains at least six copies of a DR5BD that binds DR5. In some embodiments, the fusion protein contains six or more copies of a DR5BD that binds DR5.

Multivalent DR5 binding fusion proteins of the present disclosure are capable of inducing direct cell death of damaged, transformed, virally infected, or neoplastic cells without the need for exogenous crosslinking agents. In addition, DR5 binding fusion proteins of the present disclosure do not induce direct cell death of normal, non-transformed cells, non-virally infected or non-neoplastic cells. Importantly, the DR5BDs and fusion proteins composed thereof of the present disclosure have reduced or eliminated recognition by pre-existing antibodies directed toward single domain antibodies present in some human subjects.

TAS266 is a tetravalent humanized DR5-targeting nanobody-based therapeutic, which displays superior apoptosis inducing capacity compared to bivalent antibodies, without the need for additional crosslinking by FcγRs. (Huet, H. A., et al., Multivalent nanobodies targeting death receptor 5 elicit superior tumor cell killing through efficient caspase induction. mAbs Vol. 6, Iss. 6, 2014).

It has previously been predicted that approximately half of healthy human subjects have pre-existing antibodies recognizing human single domain antibodies, known as human anti-VH autoantibodies (HAVH), which target an epitope within human VH domains (Holland et al. J Clin Immunol (2013) 33:1192-1203)). Thus, it expected that humanized camelid-derived VHHs would also be recognized by HAVH autoantibodies as the target epitope seems to be cryptic and located within human germline framework regions. The interaction of HAVH autoantibodies (also called anti-drug antibodies (ADA) or anti-single domain antibodies (ASDA), herein) can cause enhanced clustering and activation. In agreement with this hypothesis, in a Phase I clinical trial, administration of TAS266 induced elevated AST and ALT levels indicative of hepatotoxicity. Elevated enzyme levels occurred in 3 out of 4 patients leading to termination of the TAS266 trial. It was noted that the 3 patients exhibiting clinical signs of hepatotoxicity had pre-existing ADA leading trial investigators to suspect that ADA-induced hyper-clustering of the DR5 receptor causing toxicity. It was noted that the one patient without ADA had no signs of toxicity (Isaacs R, Bilic S, Kentsch K, Huet H A, Hofmann M, Rasco D, Kundamal N, Tang Z, Cooksey J, Mahipal A. Unexpected hepatotoxicity in a phase I study of TAS266, a novel tetravalent agonistic Nanobody® targeting the DR5 receptor. Papadopoulos KP1, Cancer Chemother Pharmacol. 2015 May; 75(5):887-95. doi: 10.1007/s00280-015-2712-0. Epub 2015 February 27.). In support of this idea, it has been well-documented that aggregated forms of DR5 agonists induce hepatotoxicity whereas non-aggregated forms do not (J Lemke, S von Karstedt, J Zinngrebe and H Walczak. Getting TRAIL back on track for cancer therapy. Cell Death and Differentiation (2014) 21, 1350-1364).

In some embodiments, the fusion protein contains at least one DR5BD that comprises an amino acid sequence selected from the group consisting of SEQ ID NO: 15-91. In some embodiments, the fusion protein contains two or more copies of a DR5BD that comprises an amino acid sequence selected from the group consisting of SEQ ID NO: 15-91. In some embodiments, the fusion protein contains three or more copies of a DR5BD that comprises an amino acid sequence selected from the group consisting of SEQ ID NO: 15-91. In some embodiments, the fusion protein contains four or more copies of a DR5BD that comprises an amino acid sequence selected from the group consisting of SEQ ID NO: 15-91. In some embodiments, the fusion protein contains five or more copies of a DR5BD that comprises an amino acid sequence selected from the group consisting of SEQ ID NO: 15-91. In some embodiments, the fusion protein contains six or more copies of a DR5BD that comprises an amino acid sequence selected from the group consisting of SEQ ID NO: 15-91.

In some embodiments, the fusion protein contains at least one DR5BD that comprises a complementarity determining region 1 (CDR1) comprising an amino acid sequence selected from the group consisting of SEQ ID NO: 31, 128, 134, 138, 141, 142, 159, 162, 163, 168, 173, 176, 178, 181, and 188; a complementarity determining region 2 (CDR2) comprising an amino acid sequence selected from the group consisting of SEQ ID NO: 28, 129, 131-133, 135, 137, 139, 143, 160, 164, 166, 167, 169, 171, 172, 174, 177, 179, 182, 184, 185, and 189; and a complementarity determining region 3 (CDR3) comprising an amino acid sequence selected from the group consisting of SEQ ID NO: 130, 136, 140, 144-158, 161, 165, 170, 175, 180, 183, 186, 187, and 190. In some embodiments, the fusion protein contains two or more copies of a DR5BD that comprises a CDR1 comprising an amino acid sequence selected from the group consisting of SEQ ID NO: 31, 128, 134, 138, 141, 142, 159, 162, 163, 168, 173, 176, 178, 181, and 188; a CDR2 comprising an amino acid sequence selected from the group consisting of SEQ ID NO: 28, 129, 131-133, 135, 137, 139, 143, 160, 164, 166, 167, 169, 171, 172, 174, 177, 179, 182, 184, 185, and 189; and a CDR3 comprising an amino acid sequence selected from the group consisting of SEQ ID NO: 130, 136, 140, 144-158, 161, 165, 170, 175, 180, 183, 186, 187, and 190. In some embodiments, the fusion protein contains three or more copies of a DR5BD that comprises a CDR1 comprising an amino acid sequence selected from the group consisting of SEQ ID NO: 31, 128, 134, 138, 141, 142, 159, 162, 163, 168, 173, 176, 178, 181, and 188; a CDR2 comprising an amino acid sequence selected from the group consisting of SEQ ID NO: 28, 129, 131-133, 135, 137, 139, 143, 160, 164, 166, 167, 169, 171, 172, 174, 177, 179, 182, 184, 185, and 189; and a CDR3 comprising an amino acid sequence selected from the group consisting of SEQ ID NO: 130, 136, 140, 144-158, 161, 165, 170, 175, 180, 183, 186, 187, and 190. In some embodiments, the fusion protein contains four or more copies of a DR5BD that comprises a CDR1 comprising an amino acid sequence selected from the group consisting of SEQ ID NO: 31, 128, 134, 138, 141, 142, 159, 162, 163, 168, 173, 176, 178, 181, and 188; a CDR2 comprising an amino acid sequence selected from the group consisting of SEQ ID NO: 28, 129, 131-133, 135, 137, 139, 143, 160, 164, 166, 167, 169, 171, 172, 174, 177, 179, 182, 184, 185, and 189; and a CDR3 comprising an amino acid sequence selected from the group consisting of SEQ ID NO: 130, 136, 140, 144-158, 161, 165, 170, 175, 180, 183, 186, 187, and 190. In some embodiments, the fusion protein contains five or more copies of a DR5BD that comprises a CDR1 comprising an amino acid sequence selected from the group consisting of SEQ ID NO: 31, 128, 134, 138, 141, 142, 159, 162, 163, 168, 173, 176, 178, 181, and 188; a CDR2 comprising an amino acid sequence selected from the group consisting of SEQ ID NO: 28, 129, 131-133, 135, 137, 139, 143, 160, 164, 166, 167, 169, 171, 172, 174, 177, 179, 182, 184, 185, and 189; and a CDR3 comprising an amino acid sequence selected from the group consisting of SEQ ID NO: 130, 136, 140, 144-158, 161, 165, 170, 175, 180, 183, 186, 187, and 190. In some embodiments, the fusion protein contains six or more copies of a DR5BD that comprises a CDR1 comprising an amino acid sequence selected from the group consisting of SEQ ID NO: 31, 128, 134, 138, 141, 142, 159, 162, 163, 168, 173, 176, 178, 181, and 188; a CDR2 comprising an amino acid sequence selected from the group consisting of SEQ ID NO: 28, 129, 131-133, 135, 137, 139, 143, 160, 164, 166, 167, 169, 171, 172, 174, 177, 179, 182, 184, 185, and 189; and a CDR3 comprising an amino acid sequence selected from the group consisting of SEQ ID NO: 130, 136, 140, 144-158, 161, 165, 170, 175, 180, 183, 186, 187, and 190.

In some embodiments, the fusion protein contains at least one DR5BD that comprises an amino acid sequence selected from the group consisting of SEQ ID NO: 15-91 and at least one immunoglobulin Fc region polypeptide comprising an amino acid sequence selected from the group consisting of SEQ ID NOs: 1-5 or 127. In some embodiments, the fusion protein contains two or more copies of a DR5BD that comprises an amino acid sequence selected from the group consisting of SEQ ID NO: 15-91 and at least one immunoglobulin Fc region polypeptide comprising an amino acid sequence selected from the group consisting of SEQ ID NOs: 1-5 or 127. In some embodiments, the fusion protein contains three or more copies of a DR5BD that comprises an amino acid sequence selected from the group consisting of SEQ ID NO: 15-91 and at least one immunoglobulin Fc region polypeptide comprising an amino acid sequence selected from the group consisting of SEQ ID NOs: 1-5 or 127. In some embodiments, the fusion protein contains four or more copies of a DR5BD that comprises an amino acid sequence selected from the group consisting of SEQ ID NO: 15-91 and at least one immunoglobulin Fc region polypeptide comprising an amino acid sequence selected from the group consisting of SEQ ID NOs: 1-5 or 127. In some embodiments, the fusion protein contains five or more copies of a DR5BD that comprises an amino acid sequence selected from the group consisting of SEQ ID NO: 15-91 and at least one immunoglobulin Fc region polypeptide comprising an amino acid sequence selected from the group consisting of SEQ ID NOs: 1-5 or 127. In some embodiments, the fusion protein contains six or more copies of a DR5BD that comprises an amino acid sequence selected from the group consisting of SEQ ID NO: 15-91 and at least one immunoglobulin Fc region polypeptide comprising an amino acid sequence selected from the group consisting of SEQ ID NOs: 1-5 or 127.

In some embodiments, the fusion protein contains at least one DR5BD that comprises a CDR1 comprising an amino acid sequence selected from the group consisting of SEQ ID NO: 31, 128, 134, 138, 141, 142, 159, 162, 163, 168, 173, 176, 178, 181, and 188; a CDR2 comprising an amino acid sequence selected from the group consisting of SEQ ID NO: 28, 129, 131-133, 135, 137, 139, 143, 160, 164, 166, 167, 169, 171, 172, 174, 177, 179, 182, 184, 185, and 189; and a CDR3 comprising an amino acid sequence selected from the group consisting of SEQ ID NO: 130, 136, 140, 144-158, 161, 165, 170, 175, 180, 183, 186, 187, and 190; and at least one immunoglobulin Fc region polypeptide comprising an amino acid sequence selected from the group consisting of SEQ ID NOs: 1-5 or 127. In some embodiments, the fusion protein contains two or more copies of a DR5BD that comprises a CDR1 comprising an amino acid sequence selected from the group consisting of SEQ ID NO: 31, 128, 134, 138, 141, 142, 159, 162, 163, 168, 173, 176, 178, 181, and 188; a CDR2 comprising an amino acid sequence selected from the group consisting of SEQ ID NO: 28, 129, 131-133, 135, 137, 139, 143, 160, 164, 166, 167, 169, 171, 172, 174, 177, 179, 182, 184, 185, and 189; and a CDR3 comprising an amino acid sequence selected from the group consisting of SEQ ID NO: 130, 136, 140, 144-158, 161, 165, 170, 175, 180, 183, 186, 187, and 190; and at least one immunoglobulin Fc region polypeptide comprising an amino acid sequence selected from the group consisting of SEQ ID NOs: 1-5 or 127. In some embodiments, the fusion protein contains three or more copies of a DR5BD that comprises a CDR1 comprising an amino acid sequence selected from the group consisting of SEQ ID NO: 31, 128, 134, 138, 141, 142, 159, 162, 163, 168, 173, 176, 178, 181, and 188; a CDR2 comprising an amino acid sequence selected from the group consisting of SEQ ID NO: 28, 129, 131-133, 135, 137, 139, 143, 160, 164, 166, 167, 169, 171, 172, 174, 177, 179, 182, 184, 185, and 189; and a CDR3 comprising an amino acid sequence selected from the group consisting of SEQ ID NO: 130, 136, 140, 144-158, 161, 165, 170, 175, 180, 183, 186, 187, and 190; and at least one immunoglobulin Fc region polypeptide comprising an amino acid sequence selected from the group consisting of SEQ ID NOs: 1-5 or 127. In some embodiments, the fusion protein contains four or more copies of a DR5BD that comprises a CDR1 comprising an amino acid sequence selected from the group consisting of SEQ ID NO: 31, 128, 134, 138, 141, 142, 159, 162, 163, 168, 173, 176, 178, 181, and 188; a CDR2 comprising an amino acid sequence selected from the group consisting of SEQ ID NO: 28, 129, 131-133, 135, 137, 139, 143, 160, 164, 166, 167, 169, 171, 172, 174, 177, 179, 182, 184, 185, and 189; and a CDR3 comprising an amino acid sequence selected from the group consisting of SEQ ID NO: 130, 136, 140, 144-158, 161, 165, 170, 175, 180, 183, 186, 187, and 190; and at least one immunoglobulin Fc region polypeptide comprising an amino acid sequence selected from the group consisting of SEQ ID NOs: 1-5 or 127. In some embodiments, the fusion protein contains five or more copies of a DR5BD that comprises a CDR1 comprising an amino acid sequence selected from the group consisting of SEQ ID NO: 31, 128, 134, 138, 141, 142, 159, 162, 163, 168, 173, 176, 178, 181, and 188; a CDR2 comprising an amino acid sequence selected from the group consisting of SEQ ID NO: 28, 129, 131-133, 135, 137, 139, 143, 160, 164, 166, 167, 169, 171, 172, 174, 177, 179, 182, 184, 185, and 189; and a CDR3 comprising an amino acid sequence selected from the group consisting of SEQ ID NO: 130, 136, 140, 144-158, 161, 165, 170, 175, 180, 183, 186, 187, and 190; and at least one immunoglobulin Fc region polypeptide comprising an amino acid sequence selected from the group consisting of SEQ ID NOs: 1-5 or 127. In some embodiments, the fusion protein contains six or more copies of a DR5BD that comprises a CDR1 comprising an amino acid sequence selected from the group consisting of SEQ ID NO: 31, 128, 134, 138, 141, 142, 159, 162, 163, 168, 173, 176, 178, 181, and 188; a CDR2 comprising an amino acid sequence selected from the group consisting of SEQ ID NO: 28, 129, 131-133, 135, 137, 139, 143, 160, 164, 166, 167, 169, 171, 172, 174, 177, 179, 182, 184, 185, and 189; and a CDR3 comprising an amino acid sequence selected from the group consisting of SEQ ID NO: 130, 136, 140, 144-158, 161, 165, 170, 175, 180, 183, 186, 187, and 190; and at least one immunoglobulin Fc region polypeptide comprising an amino acid sequence selected from the group consisting of SEQ ID NOs: 1-5 or 127.

In some embodiments, the fusion protein comprises an amino acid sequence selected from the group consisting of SEQ ID NOs: 92-124. In some embodiments, the fusion protein comprises an amino acid sequence selected from the group consisting of SEQ ID NOs: 92-118. In some embodiments, the fusion protein comprises an amino acid sequence selected from the group consisting of SEQ ID NOs: 119-124.

The fusion proteins of the present disclosure are capable of enhanced clustering of TNFRSF members compared to non-cross-linked bivalent antibodies. The enhanced clustered of TNFRSF members mediated by the fusion proteins of the present disclosure induce enhanced TNFRSF-dependent signaling compared to non-cross-linked bivalent antibodies. In most embodiments, the fusion protein will incorporate more than 2 DR5BDs, for example, three, four, five, or six. In some embodiments the fusion protein will incorporate DR5BDs and a binding domain directed toward non-TNFRSF member antigen. In these embodiments, the interaction of the non-TNFRSF antigen is capable of providing the additional crosslinking function and TNFRSF activation is achieved with only one or two DR5BDs. In these embodiments, the fusion protein is multispecific, binding two distinct antigens. In other embodiments, the fusion protein incorporates three or more DR5BDs and a binding domain directed toward an antigen other than DR5, wherein the interaction with this additional antigen dose not enhance DR5 clustering beyond what is achieved by the DR5BD containing portion alone, but rather provides a biodistribution advantage, focusing the DR5 agonistic activity of the fusion protein to a specific site within a subject. For example, a tetravalent DR5 binding fusion protein of the present disclosure may include an additional antigen binding domain that focuses activity to a specific site, yet does not enhance the agonistic activity beyond that achieved by a tetravalent DR5 binding fusion protein lacking this additional antigen binding domain.

In some embodiments, DR5BDs of the present disclosure are derived from antibodies or antibody fragments including scFv, Fabs, single domain antibodies (sdAb), V_{NAR}, or VHHs. In preferred embodiments the DR5BDs are human or humanized sdAb. The sdAb fragments, can be derived from VHH, V_{NAR}, engineered VH or VK domains. VHHs can be generated from camelid heavy chain only antibodies. V_{NAR}s can be generated from cartilaginous fish heavy chain only antibodies. Various methods have been implemented to generate monomeric sdAbs from conventionally heterodimeric VH and VK domains, including interface engineering and selection of specific germline families. In other embodiments, the DR5BDs are derived from non-antibody scaffold proteins for example but not limited to designed ankyrin repeat proteins (darpins), avimer, anticalin/lipocalins, centyrins and fynomers.

Generally the fusion proteins of the present disclosure consist of at least two or more DR5BDs operably linked via a linker polypeptide. The utilization of sdAb fragments as the specific DR5BD within the fusion the present disclosure has the benefit of avoiding the heavy chain: light chain mis-pairing problem common to many bi/multispecific antibody approaches. In addition, the fusion proteins of the present disclosure avoid the use of long linkers necessitated by many bispecific antibodies.

In some embodiments, all of the DR5BDs of the fusion protein recognize the same epitope on DR5. For example, the fusion proteins of present disclosure may incorporate 2, 3, 4, 5, or 6 DR5BDs with distinct recognition specificities toward various epitopes on DR5. In these embodiments, the fusion proteins of the present disclosure with contain multiple DR5BDs that target distinct regions of DR5. In some embodiments, the DR5BDs may recognize different epitopes on DR5 or recognize epitopes on DR5 and a distinct antigen. For example, the present disclosure provides multispecific fusion proteins incorporating DR5BDs that bind DR5 and at least a second antigen.

In some embodiments, the fusion protein of the present disclosure is composed of a single polypeptide. In other embodiments, the fusion protein of the present disclosure is composed of more than one polypeptide. For example, wherein a heterodimerization domain is incorporated into the fusion protein so as the construct an asymmetric fusion protein. For example if an immunoglobulin Fc region is incorporated into the fusion protein the CH3 domain can be used as homodimerization domain, or the CH3 dimer interface region can be mutated so as to enable heterodimerization.

In some embodiments, the fusion protein contains the DR5BDs opposite ends. For example the DR5BDs are located on both the amino-terminal (N-terminal) portion of the fusion protein and the carboxy-terminal (C-terminal) portion of the fusion protein. In other embodiments, all the DR5BDs reside on the same end of the fusion protein. For example, DR5BDs reside on either the amino or carboxyl terminal portions of the fusion protein.

In some embodiments, the fusion protein contains an immunoglobulin Fc region. In some embodiments, the immunoglobulin Fc region is an IgG isotype selected from the group consisting of IgG1 isotype, IgG2 isotype, IgG3 isotype, and IgG4 subclass.

In some embodiments, the immunoglobulin Fc region or immunologically active fragment thereof is an IgG isotype. For example, the immunoglobulin Fc region of the fusion protein is of human IgG1 isotype, having an amino acid sequence:

(SEQ ID NO: 1)

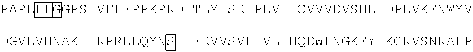

APIEKTISKA KGQPREPQVY TLPPSRDELT KNQVSLTCLV KGFYPSDIAV
EWESNGOPEN NYKTTPPVLD SDGSFFLYSK LTVDKSRWQQ GNVFSCSVMH
EALHNHYTQK SLSLSPGK

In some embodiments, the immunoglobulin Fc region or immunologically active fragment thereof comprises a human IgG1 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 1.

In some embodiments, the human IgG1 Fc region is modified at amino acid Asn297 (Boxed, Kabat Numbering) to prevent to glycosylation of the fusion protein, e.g., Asn297Ala (N297A) or Asn297Asp (N297D). In some embodiments, the Fc region of the fusion protein is modified at amino acid Leu235 (Boxed, Kabat Numbering) to alter Fc receptor interactions, e.g., Leu235Glu (L235E) or Leu235Ala (L235A). In some embodiments, the Fc region of the fusion protein is modified at amino acid Leu234 (Boxed, Kabat Numbering) to alter Fc receptor interactions, e.g., Leu234Ala (L234A). In some embodiments, the Fc region of the fusion protein is altered at both amino acid 234 and 235, e.g., Leu234Ala and Leu235Ala (L234A/L235A) or Leu234Val and Leu235Ala (L234V/L235A). In some embodiments, the Fc region of the fusion protein is altered at Gly235 to reduce Fc receptor binding. For example, wherein Gly235 is deleted from the fusion protein. In some embodiments, the human IgG1 Fc region is modified at amino acid Gly236 to enhance the interaction with CD32A, e.g., Gly236Ala (G236A). In some embodiments, the human IgG1 Fc region is lacks Lys447 (EU index of Kabat et al 1991 Sequences of Proteins of Immunological Interest).

In some embodiments, the Fc region of the fusion protein is altered at one or more of the following positions to reduce Fc receptor binding: Leu 234 (L234), Leu235 (L235), Asp265 (D265), Asp270 (D270), Ser298 (S298), Asn297 (N297), Asn325 (N325) or Ala327 (A327). For example, Leu 234Ala (L234A), Leu235Ala (L235A), Asp265Asn (D265N), Asp270Asn (D270N), Ser298Asn (S298N), Asn297Ala (N297A), Asn325Glu (N325E) or Ala327Ser (A327S). In preferred embodiments, modifications within the Fc region reduce binding to Fc-receptor-gamma receptors while have minimal impact on binding to the neonatal Fc receptor (FcRn).

In some embodiments, the Fc region of the fusion protein is lacking an amino acid at one or more of the following positions to reduce Fc receptor binding: Glu233 (E233), Leu234 (L234), or Leu235 (L235). In these embodiments, Fc deletion of these three amino acids reduces the complement protein Clq binding.

(SEQ ID NO: 2)
PAPGGPSVFL FPPKPKDTLM ISRTPEVTCV VVDVSHEDPE
VKFNWYVDGV EVHNAKTKPR EEQYNSTYRV VSVLTVLHQD
WLNGKEYKCK VSNKALPAPI EKTISKAKGQ PREPQVYTLP
PSRDELTKNQ VSLTCLVKGF YPSDIAVEWE SNGQPENNYK
TTPPVLDSDG SFFLYSKLTV DKSRWQQGNV FSCSVMHEAL
HNHYTQKSLS LSPGK

In some embodiments, the fusion or immunologically active fragment thereof comprises a human IgG2 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 2.

In some embodiments, the immunoglobulin Fc region or immunologically active fragment of the fusion protein is of human IgG2 isotype, having an amino acid sequence:

(SEQ ID NO: 3)
PAPPVAGPSV FLFPPKPKDT LMISRTPEVT CVVVDVSHED PEVQFNWYVD

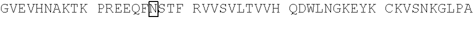

PIEKTISKTK GOPREPQVYT LPPSREEMTK NQVSLTCLVK GFYPSDISVE
WESNGOPENN YKTTPPMLDS DGSFFLYSKL TVDKSRWQQG NVFSCSVMHE
ALHNHYTOKS LSLSPGK

In some embodiments, the fusion or immunologically active fragment thereof comprises a human IgG2 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 3.

In some embodiments, the human IgG2 Fc region is modified at amino acid Asn297 (Boxed, to prevent to glycosylation of the antibody, e.g., Asn297Ala (N297A) or Asn297Asp (N297D). In some embodiments, the human IgG2 Fc region is lacks Lys447 (EU index of Kabat et al 1991 Sequences of Proteins of Immunological Interest).

In some embodiments, the immunoglobulin Fc region or immunologically active fragment of the fusion protein is of human IgG3 isotype, having an amino acid sequence:

(SEQ ID NO: 4)
PAPELLGGPS VFLFPPKPKD TLMISRTPEV TCVVVDVSHE DPEVQFKWYV

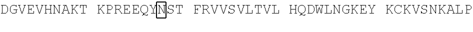

APIEKTISKT KGQPREPQVY TLPPSREEMT KNQVSLTCLV KGFYPSDIAV
EWESSGQPEN NYNTTPPMLD SDGSFFLYSK LTVDKSRWQQ GNIFSCSVMH

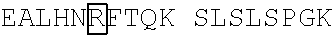

In some embodiments, the antibody or immunologically active fragment thereof comprises a human IgG3 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 4.

In some embodiments, the human IgG3 Fc region is modified at amino acid Asn297 (Boxed, Kabat Numbering) to prevent to glycosylation of the antibody, e.g., Asn297Ala (N297A) or Asn297Asp (N297D). In some embodiments, the human IgG3 Fc region is modified at amino acid 435 to extend the half-life, e.g., Arg435His (R435H). In some embodiments, the human IgG3 Fc region is lacks Lys447 (EU index of Kabat et al 1991 Sequences of Proteins of Immunological Interest).

In some embodiments, the immunoglobulin Fc region or immunologically active fragment of the fusion protein is of human IgG4 isotype, having an amino acid sequence:

(SEQ ID NO: 5)

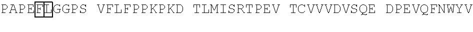

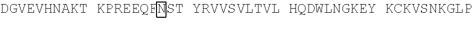

SSIEKTISKA KGQPREPQVY TLPPSQEEMT KNQVSLTCLV KGFYPSDIAV
EWESNGOPEN NYKTTPPVLD SDGSFFLYSR LTVDKSRWQE GNVFSCSVMH
EALHNHYTQK SLSLSLGK

In some embodiments, the antibody or immunologically active fragment thereof comprises a human IgG4 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 5.

In some embodiments, the immunoglobulin Fc region or immunologically active fragment of the fusion protein is of human IgG4 isotype, having an amino acid sequence:

(SEQ ID NO: 127)
PAPELLGGPS VFLFPPKPKD TLMISRTPEV TCVVVDVSQE DPEVQFNWYV

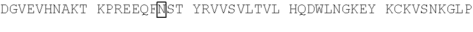

SSIEKTISKA KGOPREPQVY TLPPSQEEMT KNQVSLTCLV KGFYPSDIAV
EWESNGQPEN NYKTTPPVLD SDGSFFLYSR LTVDKSRWQE GNVFSCSVMH
EALHNHYTQK SLSLSLGK

In some embodiments, the antibody or immunologically active fragment thereof comprises a human IgG4 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 127.

In other embodiments, the human IgG4 Fc region is modified at amino acid 235 to alter Fc receptor interactions, e.g., Leu235Glu (L235E). In some embodiments, the human IgG4 Fc region is modified at amino acid Asn297 (Boxed, Kabat Numbering) to prevent to glycosylation of the antibody, e.g., Asn297Ala (N297A) or Asn297Asp (N297D). In some embodiments, the human IgG4 Fc region is lacks Lys447 (EU index of Kabat et al 1991 Sequences of Proteins of Immunological Interest).

In some embodiments, the human IgG Fc region is modified to enhance FcRn binding. Examples of Fc mutations that enhance binding to FcRn are Met252Tyr, Ser254Thr, Thr256Glu (M252Y, S254T, T256E, respectively) (Kabat numbering, Dall'Acqua et al 2006, J. Biol Chem Vol. 281(33) 23514-23524), Met428Leu and Asn434Ser (M428L, N434S) (Zalevsky et al 2010 Nature Biotech, Vol. 28(2) 157-159), or Met252Ile, Thr256Asp, Met428Leu (M252I, T256D, M428L, respectively), (EU index of Kabat et al 1991 Sequences of Proteins of Immunological Interest).

In some embodiments where the fusion protein of the disclosure includes an Fc polypeptide, the Fc polypeptide is mutated or modified. In these embodiments the mutated or modified Fc polypeptide includes the following mutations: Met252Tyr and Met428Leu or Met252Tyr and Met428Val (M252Y, M428L, or M252Y, M428V) using the Kabat numbering system.

In some embodiments, the human IgG Fc region is modified to alter antibody-dependent cellular cytotoxicity (ADCC) and/or complement-dependent cytotoxicity (CDC), e.g., the amino acid modifications described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1):1-11. Examples of mutations that enhance ADCC include modification at Ser239 and Ile332, for example Ser239Asp and Ile332Glu (S239D, I332E). Examples of mutations that enhance CDC include modifications at Lys326 and Glu333. In some embodiments the Fc region is modified at one or both of these positions, for example Lys326Ala and/or Glu333Ala (K326A and E333A) using the Kabat numbering system.

In some embodiments, the human IgG Fc region is modified to induce heterodimerization. For example, having an amino acid modification within the CH3 domain at Thr366, which when replaced with a more bulky amino acid, e.g., Try (T366W), is able to preferentially pair with a second CH3 domain having amino acid modifications to less bulky amino acids at positions Thr366, Leu368, and Tyr407, e.g., Ser, Ala and Val, respectively (T366S/L368A/Y407V). Heterodimerization via CH3 modifications can be further stabilized by the introduction of a disulfide bond, for example by changing Ser354 to Cys (S354C) and Y349 to Cys (Y349C) on opposite CH3 domains (Reviewed in Carter, 2001 Journal of Immunological Methods, 248: 7-15).

In some embodiments, the human IgG Fc region is modified to prevent dimerization. In these embodiments, the fusion proteins of the present disclosure are monomeric. For example modification at residue Thr366 to a charged residue, e.g. Thr366Lys, Thr366Arg, Thr366Asp, or Thr366Glu (T366K, T366R, T366D, or T366E, respectively), prevents CH3-CH3 dimerization.

In some embodiments, the Fc region of the fusion protein is altered at one or more of the following positions to reduce Fc receptor binding: Leu 234 (L234), Leu235 (L235), Asp265 (D265), Asp270 (D270), Ser298 (5298), Asn297 (N297), Asn325 (N325) or Ala327 (A327). For example, Leu 234Ala (L234A), Leu235Ala (L235A), Asp265Asn (D265N), Asp270Asn (D270N), Ser298Asn (S298N), Asn297Ala (N297A), Asn325Glu (N325E) or Ala327Ser (A327S). In preferred embodiments, modifications within the Fc region reduce binding to Fc-receptor-gamma receptors while have minimal impact on binding to the neonatal Fc receptor (FcRn).

In some embodiments, the fusion protein contains a polypeptide derived from an immunoglobulin hinge region. The hinge region can be selected from any of the human IgG subclasses. For example the fusion protein may contain a modified IgG1 hinge having the sequence of EPKSSDKTHTCPPC (SEQ ID NO: 6), where in the Cys220 that forms a disulfide with the C-terminal cysteine of the light chain is mutated to serine, e.g., Cys220Ser (C220S). In other embodiments, the fusion protein contains a truncated hinge having a sequence DKTHTCPPC (SEQ ID NO: 7).

In some embodiments, the fusion protein has a modified hinge from IgG4, which is modified to prevent or reduce strand exchange, e.g., Ser228Pro (S228P), having the sequence ESKYGPPCPPC (SEQ ID NO: 8). In some embodiments, the fusion protein contains linker polypeptides. In other embodiments, the fusion protein contains linker and hinge polypeptides.

In some embodiments, the fusion proteins of the present disclosure lack or have reduced Fucose attached to the N-linked glycan-chain at N297. There are numerous ways to prevent fucosylation, including but not limited to production in a FUT8 deficient cell line; addition inhibitors to the mammalian cell culture media, for example Castanospermine; and metabolic engineering of the production cell line.

In some embodiments, the DR5BD is engineered to eliminate recognition by pre-existing antibodies found in humans. In some embodiments, single domain antibodies of the present disclosure are modified by mutation of position Leu11, for example Leu11Glu (L11E) or Leu11Lys (L11K). In other embodiments, single domain antibodies of the present disclosure are modified by changes in carboxy-terminal region, for example the terminal sequence consists of GQGTLVTVKPGG (SEQ ID NO: 9) or GQGTLVTVEPGG (SEQ ID NO: 10) or modification thereof. In some embodiments, the single domain antibodies of the present disclosure are modified by mutation of position 11 and by changes in carboxy-terminal region.

In some embodiments, the DR5BDs of the fusion proteins of the present disclosure are operably linked via amino acid linkers. In some embodiments, these linkers are composed predominately of the amino acids Glycine and Serine, denoted as GS-linkers herein. The GS-linkers of the fusion proteins of the present disclosure can be of various lengths, for example 5, 6, 7, 8, 9, 10, 11, 12, 13, 14, 15, 16, 17, 18, 19, 20 amino acids in length.

In some embodiments, the GS-linker comprises an amino acid sequence selected from the group consisting of GGSGGS, i.e., (GGS)₂ (SEQ ID NO: 11); GGSGGSGGS, i.e., (GGS)₃ (SEQ ID NO: 12); GGSGGSGGSGGS, i.e., (GGS)₄ (SEQ ID NO: 13); and GGSGGSGGSGGSGGS, i.e., (GGS)₅ (SEQ ID NO: 14).

In some embodiments, the multivalent TNFRSF binding fusion protein is tetravalent. In some embodiments, the tetravalent TNFRSF binding fusion protein has the following structure: VHH-Linker-VHH-Linker-Hinge-Fc, where the VHH is a humanized or fully human VHH sequence that binds at least DR5.

In some embodiments, the multivalent TNFRSF binding fusion protein is tetravalent. In some embodiments, the tetravalent TNFRSF binding fusion protein has the following structure: DR5BD-Linker-DR5BD-Linker-Hinge-Fe, where the DR5BD is a humanized or fully human VHH sequence.

In some embodiments, the multivalent TNFRSF binding fusion protein is hexavalent. In some embodiments, the hexavalent TNFRSF binding fusion protein has the following structure: VHH-Linker-VHH-Linker-VHH-Linker-Hinge-Fc, where the VHH is a humanized or fully human VHH sequence that binds at least DR5.

In some embodiments, the multivalent TNFRSF binding fusion protein is hexavalent. In some embodiments, the hexavalent TNFRSF binding fusion protein has the following structure: DR5BD-Linker-DR5BD-Linker-DR5BD-Linker-Hinge-Fc, where the DR5BD is a humanized or fully human VHH sequence.

In some embodiments, the multivalent fusion proteins targeting DR5 of the present disclosure are operably linked via amino acid linkers. In some embodiments, these linkers are composed predominately of the amino acids Glycine and Serine, denoted as GS-linkers herein. The GS-linkers of the fusion proteins of the present disclosure can be of various lengths, for example 5, 6, 7, 8, 9, 10, 11, 12, 13, 14, 15, 16, 17, 18, 19, 20 amino acids in length.

In some embodiments, the GS-linker comprises an amino acid sequence selected from the group consisting of GGSGGS, i.e., (GGS)₂ (SEQ ID NO: 11); GGSGGSGGS, i.e., (GGS)₃ (SEQ ID NO: 12); GGSGGSGGSGGS, i.e., (GGS)₄ (SEQ ID NO: 13); and GGSGGSGGSGGSGGS, i.e., (GGS)₅ (SEQ ID NO: 14).

In some embodiments, the multivalent DR5 binding fusion protein is tetravalent. In some embodiments, the tetravalent DR5 binding fusion protein has the following structure: VHH-Linker-VHH-Linker-Hinge-Fc, where the VHH is a humanized or fully human VHH sequence. In some embodiments, the VHH sequence is selected from the group consisting of SEQ ID NO: 15-91. In some embodiments, the tetravalent DR5 binding fusion protein comprises an amino acid sequence selected from the group consisting of SEQ ID NOs: 92-118.

In some embodiments, the multivalent DR5 binding fusion protein is hexavalent. In some embodiments, the hexavalent DR5 binding fusion protein has the following structure: VHH-Linker-VHH-Linker-VHH-Linker-Hinge-Fc, where the VHH is a humanized or fully human VHH sequence. In some embodiments, the VHH sequence is selected from the group consisting of SEQ ID NO: 15-91. In some embodiments, the hexavalent DR5 binding fusion protein comprises an amino acid sequence selected from the group consisting of SEQ ID NOs: 119-124.

### BRIEF DESCRIPTION OF FIGURES

FIG. 1 is schematic of exemplary multivalent and multispecific fusion proteins of the present disclosure.

FIGS. 2A, 2B, 2C, 2D, 2E, 2F, and 2G are a series of graphs demonstrating the binding of representative DR5 VHHs or humanized variants thereof to either human DR5 (FIGS. 2A, 2B, 2E, 2F, and 2G) and cyno DR5 (FIGS. 2C and 2D). FIGS. 2A, 2B, 2E, 2F, and 2G demonstrate the binding of some VHHs and humanized VHHs binding to human DR5 as assessed by flow cytometry on DR5 expressing CHO cells. FIGS. 2C and 2D demonstrate the binding of VHHs and humanized VHHs binding to cyno DR5 as assessed by ELISA using recombinant cyno DR5. In FIGS. 2A, B, C, D and E, the DR5 targeting fusion proteins used were bivalent, and formats used were VHH-Fc or humanized (hz) hzVHH-Fc. In FIGS. 2F and 2G, humanized tetravalent (VHH-linker-VHH-Fc) DR5 targeting Fc-fusion proteins were used.

FIGS. 3A, 3B, and 3C are a series of graphs demonstrating the direct apoptosis inducing capacity of DR5 targeting fusion proteins of the present disclosure. In all assays, the Colo205 cells were used and the DR5 targeting VHH was H10 formatted as (A) H10-Fc (bivalent), (B) H10-linker-H10-Fc (tetravalent), or (C) H10-linker-H10-linker-H10-Fc (hexavalent). FIG. 3A is a graph demonstrating the enhanced apoptosis inducing capacity of a bivalent DR5 targeting fusion protein when a crosslinking agent is used. FIG. 3B is a graph demonstrating the enhanced apoptosis inducing capacity of a tetravalent DR5 targeting fusion protein compared to a bivalent DR5 targeting fusion protein. FIG. 3C is a graph demonstrating the enhanced apoptosis inducing capacity of a tetravalent and furthermore hexavalent DR5 targeting fusion protein compared to a bivalent DR5 targeting fusion protein and TRAIL.

FIG. 4A is a graph demonstrating the ability of a hexavalent DR5 targeting fusion protein to induce apoptosis of the resistant cell line Panc-1. Colo205 is shown for comparison. H10 DR5 targeting VHH is shown.

FIG. 4B is a graph demonstrating the enhanced sensitivity of Panc-1 to a tetravalent DR5 targeting fusion protein when doxorubicin is added. The DR5 targeting VHH shown is humanized F03 (hzF03), formatted as hzF03-linker-hzF03-Fc.

FIG. 5 is a graph demonstrating the anti-tumor activity of tetravalent DR5 targeting fusion proteins of the present disclosure in a murine tumor xenograft model with Colo-205 cells. Fusion proteins were dosed at 1 mg/kg weekly for 4 weeks via IV administration. Dosing began when tumors reached approximately 300 mm³.

FIGS. 6A, 6B, 6C, 6D, 6E, 6F, 6G, 6H, 6I, and 6J are a series of graphs demonstrating direct cell death inducing capacity of some the tetravalent DR5 targeting fusion proteins of the present disclosure compared to TAS266 (a tetravalent DR5 nanobody described in PCT Publication No. WO 2011/098520A1) on various cancer cell lines (FIGS. 6A and 6B) Colo-205, Panc-1 (FIGS. 6C and 6J), JL-1 (FIG. 6D), HCT-116 (FIG. 6E), NCI-H28 (FIG. 6F), NCI-H460 (FIG. 6G), HT-29 (FIG. 6H), and MSTO-211H (FIG. 6I). In FIGS. 6A-6D, the DR5 targeting VHH is a humanized variant of 1F5 (hz1F5) formatted as hzVHH-linker-hzVHH-Fc. In FIGS. 6E-6J, the DR5 targeting VHH is a humanized variant of either 1F2 (hz1F2) or 2C6 (hz2C6) formatted as hzVHH-linker-hzVHH-Fc variants.

FIG. 7A is a graph demonstrating the differences in autoantibody recognition of TAS266, (a tetravalent DR5 nanobody described in PCT Publication No. WO 2011/098520A1) and humanized tetravalent 1F5 (Tet-hz1F5v5) of present disclosure. This graph depicts the results from the serum of 45 human donors. Autoantibodies containing either a kappa or lambda light chain were detected in separate assays using the respective anti-human Ig Kappa or anti-human Ig Lambda HRP-conjugated secondary antibodies. Data are normalized to positive control of an IgG antibody having either a lambda or kappa light chain, respectively. TAS266 displays significant autoantibody recognition while autoantibody recognition of Tet-hz1F5v5 is reduced to that of IgG control background.

FIG. 7B is a graph that demonstrates that pooled serum from multiple human donors (IVIG, Gamunex®-C, Grifols) contains some IgG antibodies that recognize single domain antibodies (sdAb) including TAS266.

FIG. 7C is a graph that demonstrates that recognition of TAS266 by autoantibodies within IVIG induces apoptosis of primary human hepatocytes.

FIGS. 8A and 8B are a series of graphs demonstrating the autoantibody recognition-dependent hepatotoxicity of TAS266, but not Tet-hz1F5v5, on HepRG™ the terminally differentiated hepatic cells derived from a hepatic progenitor cell line. In FIG. 8A, apoptosis was monitored using a caspase-3/7-specific fluorogenic substrate with IncuCyte Zoom live cell imager (Essen Biosciences), data shown is at 48 hours. In FIG. 8B, apoptosis was monitored after 48 hours using a CellTiter Glo assay (Promega). IVIG (Gamunex®-C, Grifols) was used an sdAb-directed autoantibody containing antibody pool.

FIGS. 9A, 9B, 9C, and 9D are a series of graphs demonstrating the autoantibody recognition-dependent hepatoxicity of TAS266, but not the tetravalent DR5 targeting fusion proteins of the present disclosure. HepRG™ cells were used as a surrogate for human hepatocytes. IVIG (Gamunex®-C, Grifols) was used an sdAb-directed autoantibody containing antibody pool. FIGS. 9A and 9C depict independent 48 hour assays and demonstrate that TAS266 induces hepatotoxicity when crosslinked by autoantibodies. Moderate hepatotoxicity was observed when a crosslinking anti-human Fc secondary was added to the tetravalent DR5 targeting fusion proteins of the present disclosure, hz1F5, hz1F2 or hz2C6, formatted as hzVHH-linker-hzVHH-Fc. Cell viability was assessed by CellTiter Glo (Promega). FIG. 9D depicts the reduction autoantibody recognition-dependent hepatotoxicity of TAS266 when it is modified at amino acid positions Leu11 and the C-terminal region of each of the four DR5 sdAbs (FIX-TAS266, SEQ ID NO: 126). This data demonstrate that hepatocytoxicity of FIX-266 in the presence of IVIG is reduced to that of TAS266 in the absence of IVIG. HepRG cell viability was assessed by CellTiter Glo (Promega). FIG. 9B depicts the kinetics of autoantibody recognition-dependent hepatotoxicity of TAS266 as well as the secondary antibody crosslinking-dependent hepatotoxicity of Tet-hz1F5v6. Apoptosis was monitored over a 46 hour period using a caspase-3/7-specific fluorogenic substrate with IncuCyte Zoom live cell imager (Essen Biosciences). Tetravalent DR5 targeting fusion proteins of the present disclosure do not induce hepatotoxicity in the presence or absence of sdAb-directed autoantibody containing antibodies.

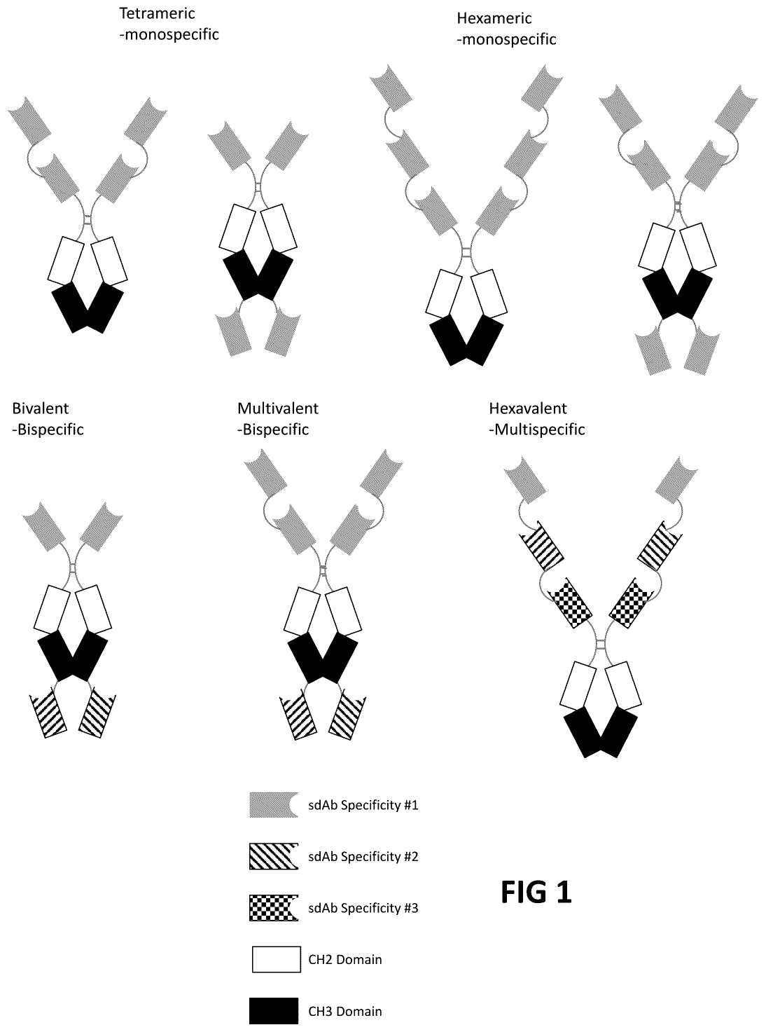

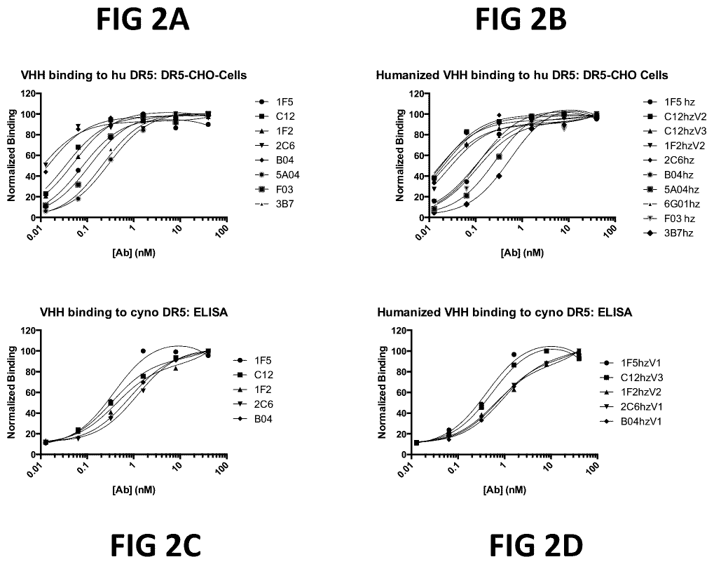

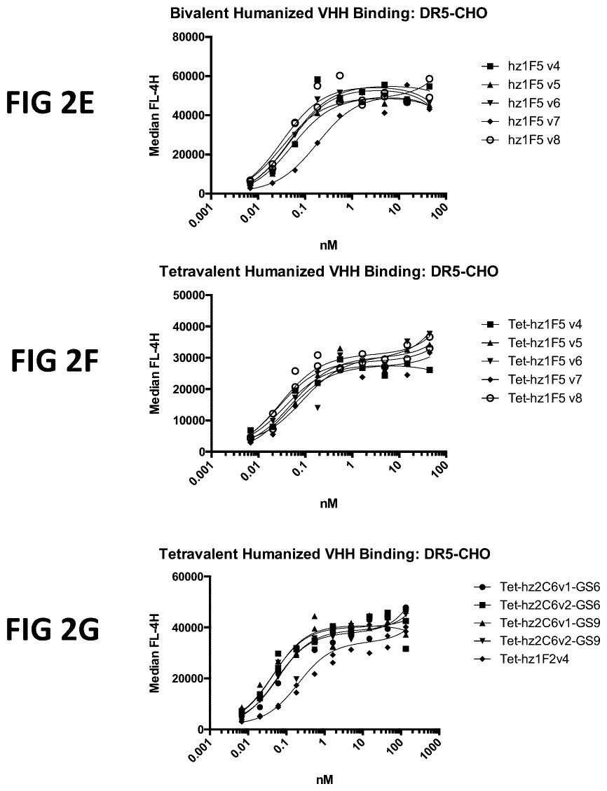

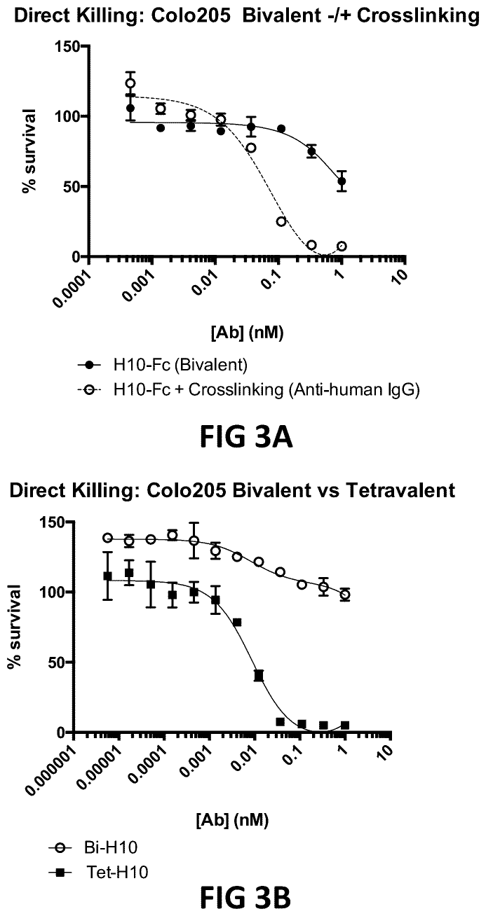

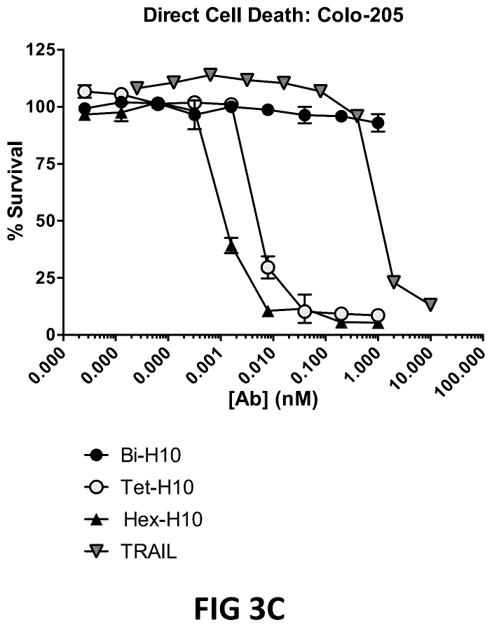

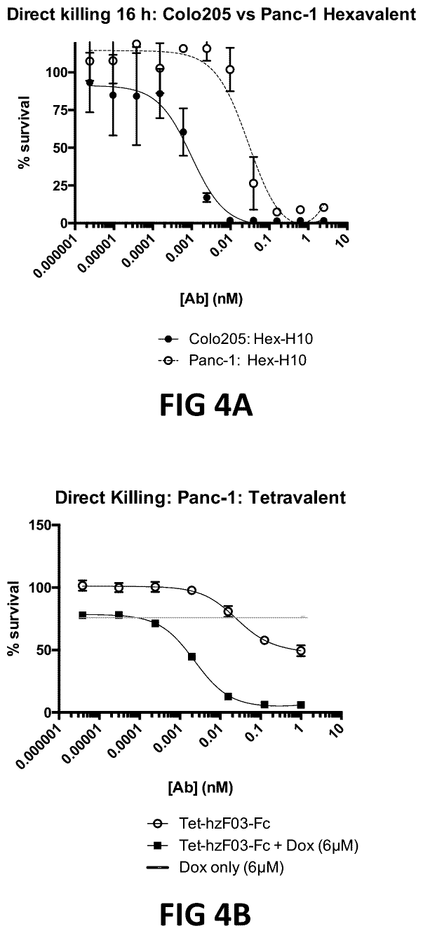

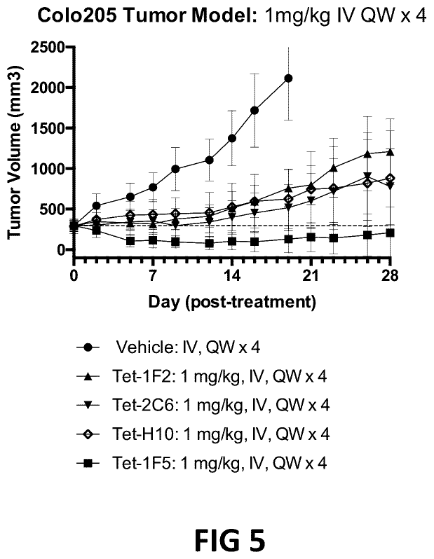

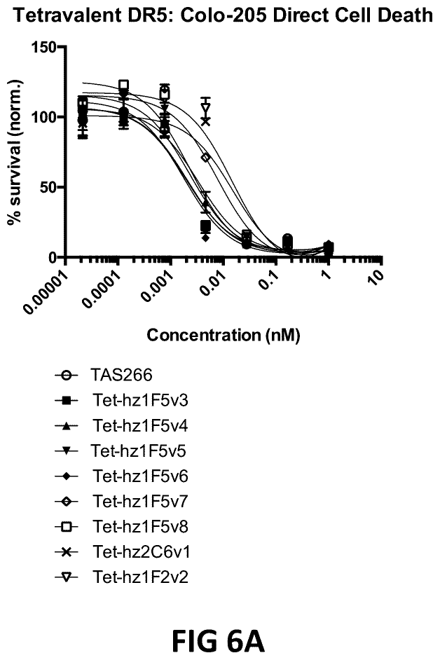

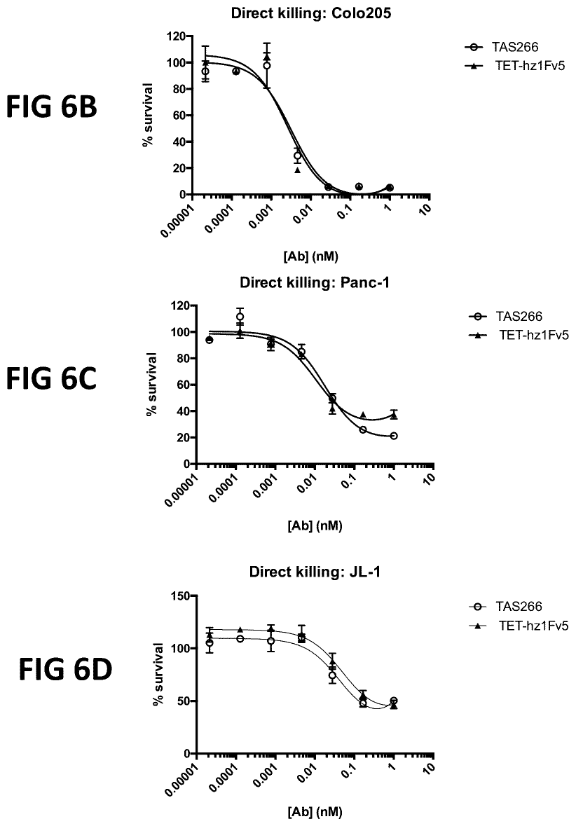

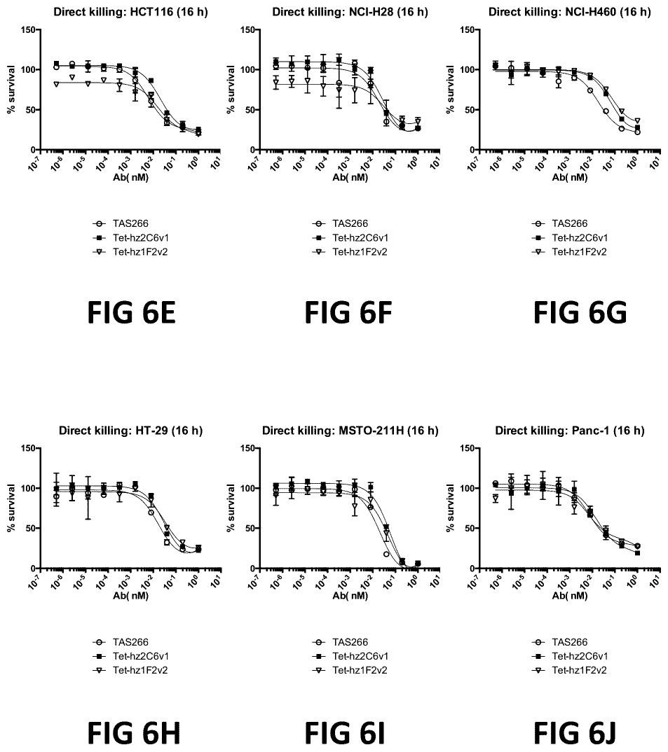

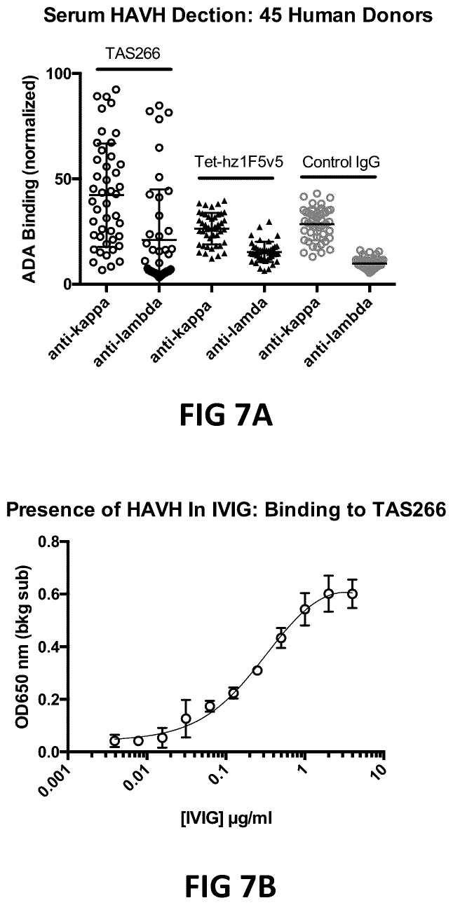

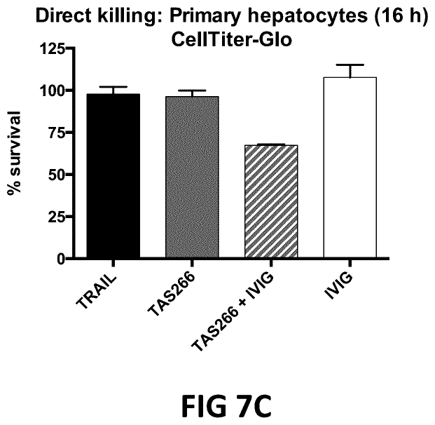

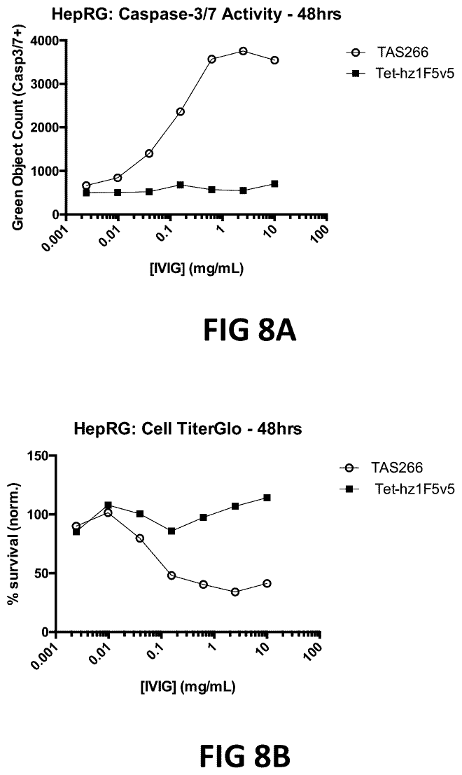

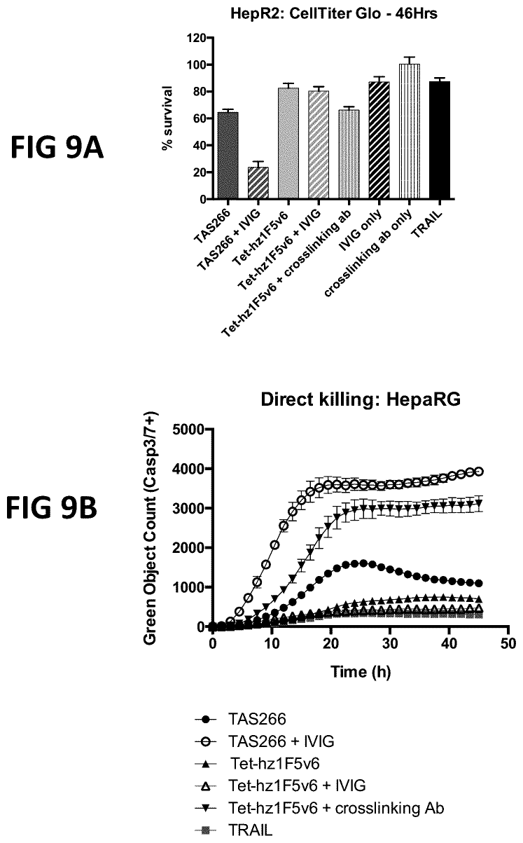

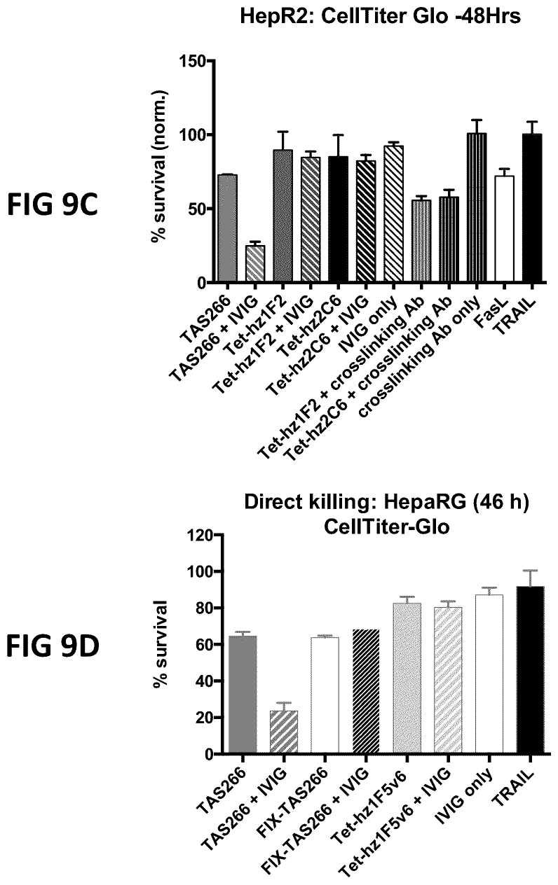

### DETAILED DESCRIPTION

The disclosure provides molecules that specifically engage death receptor 5 (DR5), a member of the TNF receptor superfamily (TNFRSF). More specifically this disclosure relates to multivalent molecules that bind at least DR5. These multivalent TNFRSF binding fusion proteins comprise two or more TNFRSF binding domains (DR5BDs), where at least one DR5BD binds DR5. These molecules are referred to herein as DR5-targeting molecules.

These DR5-targeting molecules include at least one copy of a single-domain antibody (sdAb) sequence that specifically binds DR5. In some embodiments, the DR5-targeting molecules include two or more copies of a sdAb that specifically binds DR5, for example, three or more, four or more, five or more, or six or more copies of a sdAb that specifically binds DR5.

A single-domain antibody (sdAb) is an antibody fragment consisting of a single monomeric variable antibody domain that is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain).

Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and/or bovine. In some embodiments, a single domain antibody as used herein is a naturally occurring single domain antibody known as heavy chain antibody devoid of light chains. For clarity reasons, this variable domain derived from a heavy chain antibody naturally devoid of light chain is known herein as a VHH to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the disclosure.

A single-domain antibody can be obtained by immunization of dromedaries, camels, llamas, alpacas or sharks with the desired antigen and subsequent isolation of the mRNA coding for heavy-chain antibodies. By reverse transcription and polymerase chain reaction, a gene library of single-domain antibodies containing several million clones is produced. Screening techniques like phage display and ribosome display help to identify the clones binding the antigen. (See e.g., Arbabi Ghahroudi, M.; Desmyter, A.; et al. (1997). “Selection and identification of single domain antibody fragments from camel heavy-chain antibodies”. FEBS Letters 414 (3): 521-526.)

A different method uses gene libraries from animals that have not been immunized beforehand. Such naïve libraries usually contain only antibodies with low affinity to the desired antigen, making it necessary to apply affinity maturation by random mutagenesis as an additional step. (Saerens, D.; et al. (2008). “Single-domain antibodies as building blocks for novel therapeutics”. Current Opinion in Pharmacology 8 (5): 600-608.)

When the most potent clones have been identified, their DNA sequence is optimized, for example to improve their stability towards enzymes. Another goal is humanization to prevent immunological reactions of the human organism against the antibody. Humanization is unproblematic because of the homology between camelid VHH and human VH fragments. (See e.g., Saerens, et al., (2008). “Single-domain antibodies as building blocks for novel therapeutics”. Current Opinion in Pharmacology 8 (5): 600-608.) The final step is the translation of the optimized single-domain antibody in E. coli, Saccharomyces cerevisiae or other suitable organisms.

Single domain antibody fragments are also derived from conventional antibodies. In some embodiments, single-domain antibodies can be made from common murine or human IgG with four chains. (Holt, L. J.; et al. (2003). “Domain antibodies: proteins for therapy”. Trends in Biotechnology 21 (11): 484-490.) The process is similar, comprising gene libraries from immunized or naïve donors and display techniques for identification of the most specific antigens. A problem with this approach is that the binding region of common IgG consists of two domains (VH and VL), which tend to dimerize or aggregate because of their lipophilicity. Monomerization is usually accomplished by replacing lipophilic by hydrophilic amino acids, but often results in a loss of affinity to the antigen. (See e.g., Borrebaeck, C. A. K.; Ohlin, M. (2002). “Antibody evolution beyond Nature”. Nature Biotechnology 20 (12): 1189-90.) If affinity can be retained, the single-domain antibodies can likewise be produced in E. coli, S. cerevisiae or other organisms.

Monovalent single domain antibodies can be made multivalent via several methods. For example the cDNA encoding a first sdAb can be genetically fused to a linker encoding DNA sequence followed by a second cDNA encoding an sdAb and so forth and so on. Alternatively, the cDNA encoding an sdAb can be fused to cDNA encoding a second protein or fragment thereof that naturally multimerizes or is engineered to multimerize. For example, fusion of an sdAb to an IgG Fc region will dimerize the sdAb. Wherein a tandem sdAb encoding constructed is linked to an Fc encoding construct the resultant fusion protein once expressed will be tetravalent. Wherein a construct that encodes three sdAbs is linked to an Fc encoding construct the resultant fusion protein once expressed will be hexavalent. This disclosure contemplates the use of the additional multimerization domains, including collagen homotrimerization and heterotrimerization domains, leucine zipper domains, p53 tetramerization domains, c-Jun:Fos heterodimeric peptide sequences, cartilage oligomeric matrix protein (COMP48), trimeric adiponectin, trimeric surfactant protein D, and/or synaptic acetylcholinesterase tetramer.

### Death Receptor 5 (TRIAL-R2, TNFRSF10B) Targeting

The TNF-related apoptosis-inducing ligand (TRAIL) evolved to play critical roles in mammalian development and host defense by selectively eradicating unwanted, infected and malignant cells from healthy cell populations. On binding the TNF receptor family members DR4 or DR5, TRAIL induces cell death via caspase-dependent apoptosis. DR5 (TNFRSF10B) appears to be the primary receptor on tumor cells that facilitates the observed tumor biased activity of the TRAIL pathway. DR5 is activated by the natural ligand TRAIL, which brings three DR5 receptors within close proximity thereby activating intracellular caspase-8 and initiating activation of other death-inducing caspases, such as caspases-9 and caspases-3. Thus initiation of this cell death pathway requires clustering of DR5 receptors for efficient cell death.

Efforts to clinically exploit the TRAIL pathway for cancer therapy relied upon a recombinant version of the natural ligand TRAIL and antibodies specific for DR5. Antibody agonists targeting DR5 required a crosslinking agent in preclinical in vitro experiments. This was due to the fact the conventional antibodies resulted in clustering of only two DR5 receptors (one per each heavy and light chain). Two DR5 receptors are insufficient to activate the cell death pathway thus the need for a crosslinking agent. Surprisingly in vivo administration of DR5 targeting antibodies in pre-clinical mouse models of human cancers showed significant activity in a wide variety of tumor types. This activity was later shown to be dependent on mouse FcgammaR (FcγR) receptors. Clinical studies in humans failed to reproduce the robust responses seen in these pre-clinical mouse models. The lack of activity in humans is hypothesized to be due to insufficient antibody crosslinking. This may be due to differences in serum IgG, FcgammaR (FcγR) and or TRAIL concentrations between immune compromised mice and human cancer patients.

The present disclosure provides multivalent fusion proteins targeting DR5 that are capable of potently agonizing DR5 signaling mediating direct cell death. The fusion proteins of the present disclosure can be trivalent, tetravalent, pentavalent, or hexavalent. Importantly, the fusion proteins of the present disclosure are capable of eliciting apoptosis of DR5 expressing cells independently of exogenous crosslinking agents.

In some embodiments, the fusion proteins of the present disclosure incorporate a DR5BD that binds DR5. In preferred embodiments, the DR5 binding DR5BD does not bind DR4, decoy R1, decoy R2, Osteopontin, or any other TNFRSF member. In preferred embodiments the DR5 binding DR5BD binds human and cynomolgus monkey DR5. In some embodiments, the DR5 binding DR5BD blocks the interaction of DR5 and its ligand TRAIL. In other embodiments, the DR5 binding DR5BD does not block the interaction of DR5 and its ligand TRAIL. In some embodiments, the fusion protein of the present disclosure incorporates multiple DR5 binding DR5BDs that recognize distinct epitopes on DR5. In some embodiments, the fusion protein of the present disclosure incorporates multiple DR5 binding DR5BDs, wherein some DR5BDs block the DR5-TRAIL interaction and other do not block the DR5-TRAIL interaction. In preferred embodiments, DR5 targeting fusion proteins of the present disclosure induce direct cell death of tumor cells. The DR5 targeting fusion proteins of the present disclosure have utility in treating tumors of both hematologic and solid in nature.

Exemplary DR5 Binding sdAbs

DR5 VHH (llama-derived) and humanized sequences are shown below, and the CDR sequences are shown below each sequence. In some embodiments, the DR5 binding sdAb is fused to an IgG Fc region and in these embodiments the fusion protein is bivalent having two DR5 binding domains per molecule. In some embodiments, two DR5 binding sdAbs (2×) are fused to an IgG Fc region and in these embodiments the fusion protein is tetravalent having four DR5 binding domains per molecule. In some embodiments, three DR5 binding sdAbs (3×) are fused to an IgG Fc region and in these embodiments the fusion protein is hexavalent having six DR5 binding domains per molecule.

1F5
(SEQ ID NO: 15)
QVQLVQSGGGLVQAGDSLRLSCAASGLTFPNYGMGWFRQAPGEEREFLAVIYWSGGTVFYADSV
KGRFTISRDAAKNMVYLQMNSLKSDDTAVYYCAVTIRGAATQTWKYDYWGRGTQVTVSS
CDR1:
(SEQ ID NO: 128)
SGLTFPNYGM
CDR2:
(SEQ ID NO: 129)
VIYWSGGTVF
CDR3:
(SEQ ID NO: 130)
AVTIRGAATQTWKYDYW
hz1F5v1
(SEQ ID NO: 16)
EVQLLESGGGLVQPGGSLRLSCAASGLTFPNYGMSWFRQAPGKGLEFVSAIYWSGGTVYYADSV
KGRFTISRDNSKNTLYLQMNSLRAEDTAVYYCAVTIRGAATQTWKYDYWGQGTLVTVSS
CDR1:
(SEQ ID NO: 128)
SGLTFPNYGM
CDR2:
(SEQ ID NO: 131)
AIYWSGGTVY
CDR3:
(SEQ ID NO: 130)
AVTIRGAATQTWKYDYW
hz1F5v1opt
(SEQ ID NO: 17)
EVQLLESGGGEVQPGGSLRLSCAASGLTFPNYGMSWFRQAPGKGLEFVSAIYWSGGTVYYAESV
KGRFTISRDNAKNTLYLQMSSLRAEDTAVYYCAVTIRGAATQTWKYDYWGQGTLVTVKPGG
CDR1:
(SEQ ID NO: 128)
SGLTFPNYGM
CDR2:
(SEQ ID NO: 131)
AIYWSGGTVY
CDR3:
(SEQ ID NO: 130)
AVTIRGAATQTWKYDYW
hz1F5v1opt1
(SEQ ID NO: 18)
EVQLLESGGGEVQPGGSLRLSCAASGLTFPNYGMSWFRQAPGKGLEFVSAIYWSGGTVYYAESV
KGRFTISRDNAKNTLYLQMSSLRAEDTAVYYCAVTIRGAATQTWKYDYWGQGTLVTVKP
CDR1:
(SEQ ID NO: 128)
SGLTFPNYGM
CDR2:
(SEQ ID NO: 131)
AIYWSGGTVY
CDR3:
(SEQ ID NO: 130)
AVTIRGAATQTWKYDYW
hz1F5v2
(SEQ ID NO: 19)
EVQLLESGGGEVQPGGSLRLSCAASGLTFPNYGMSWFRQAPGKEREFVSAIYWSGGTVYYAESV
KGRFTISRDNAKNTLYLQMSSLRAEDTAVYYCAVTIRGAATQTWKYDYWGQGTQVTVKP
CDR1:
(SEQ ID NO: 128)
SGLTFPNYGM
CDR2:
(SEQ ID NO: 131)
AIYWSGGTVY
CDR3:
(SEQ ID NO: 130)
AVTIRGAATQTWKYDYW
hz1F5v1DS
(SEQ ID NO: 20)
EVQLLESGGGEVQPGGSLRLSCAASGLTFPNYGMSWFRQAPGKGLEFVCAIYWSGGTVYYAESV
KGRFTCSRDNAKNTLYLQMSSLRAEDTAVYYCAVTIRGAATQTWKYDYWGQGTLVTVKPGG
CDR1:
(SEQ ID NO: 128)
SGLTFPNYGM
CDR2:
(SEQ ID NO: 131)
AIYWSGGTVY
CDR3:
(SEQ ID NO: 130)
AVTIRGAATQTWKYDYW
hz1F5v3
(SEQ ID NO: 85)
EVQLLESGGGEVQPGGSLRLSCAASGLTFPNYGMGWFRQAPGKEREFVSAIYWSGGTVFYAESV
KGRFTISRDNAKNTVYLQMSSLRAEDTAVYYCAVTIRGAATQTWKYDYWGQGTLVTVKP
CDR1:
(SEQ ID NO: 128)
SGLTFPNYGM
CDR2:
(SEQ ID NO: 132)
AIYWSGGTVF
CDR3:
(SEQ ID NO: 130)
AVTIRGAATQTWKYDYW
hz1F5v4
(SEQ ID NO: 86)
EVQLLESGGGEVQPGGSLRLSCAASGLTFPNYGMGWFRQAPGKEREFLAVIYWSGGTVFYAESV
KGRFTISRDNAKNTVYLQMSSLRAEDTAVYYCAVTIRGAATQTWKYDYWGQGTLVTVKP
CDR1:
(SEQ ID NO: 128)
SGLTEPNYGM
CDR2:
(SEQ ID NO: 129)
VIYWSGGTVF
CDR3:
(SEQ ID NO: 130)
AVTIRGAATQTWKYDYW
hz1F5v5
(SEQ ID NO: 87)
EVQLLESGGGEVQPGGSLRLSCAASGLTFPNYGMGWFRQAPGKEREFVSAIYWSGGTVYYAESV
KGRFTISRDNAKNTLYLQMSSLRAEDTAVYYCAVTIRGAATQTWKYDYWGQGTLVTVKP
CDR1:
(SEQ ID NO: 128)
SGLTFPNYGM
CDR2:
(SEQ ID NO: 131)
AIYWSGGTVY
CDR3:
(SEQ ID NO: 130)
AVTIRGAATQTWKYDYW
hz1F5v6
(SEQ ID NO: 88)
EVQLLESGGGEVQPGGSLRLSCAASGLTFPNYGMGWFRQAPGKEREFLAVIYWSGGTVYYAESV
KGRFTISRDNAKNTLYLQMSSLRAEDTAVYYCAVTIRGAATQTWKYDYWGQGTLVTVKP
CDR1:
(SEQ ID NO: 128)
SGLTFPNYGM
CDR2:
(SEQ ID NO: 133)
VIYWSGGTVY
CDR3:
(SEQ ID NO: 130)
AVTIRGAATQTWKYDYW
hz1F5v7
(SEQ ID NO: 89)
EVQLLESGGGEVQPGGSLRLSCAASGLTFPNYGMGWFRQAPGKEREFVSAIYWSGGTVYYAESV
KGRFTISRDNAKNTVYLQMSSLRAEDTAVYYCAVTIRGAATQTWKYDYWGQGTLVTVKP
CDR1:
(SEQ ID NO: 128)
SGLTEPNYGM
CDR2:
(SEQ ID NO: 131)
AIYWSGGTVY
CDR3:
(SEQ ID NO: 130)
AVTIRGAATQTWKYDYW
hz1F5v8
(SEQ ID NO: 90)
EVQLLESGGGEVQPGGSLRLSCAASGLTFPNYGMGWFRQAPGKEREFLAVIYWSGGTVYYAESV
KGRFTISRDNAKNTVYLQMSSLRAEDTAVYYCAVTIRGAATQTWKYDYWGQGTLVTVKP
CDR1:
(SEQ ID NO: 128)
SGLTFPNYGM
CDR2:
(SEQ ID NO: 133)
VIYWSGGTVY
CDR3:
(SEQ ID NO: 130)
AVTIRGAATQTWKYDYW
2C6
(SEQ ID NO: 21)
QVQLVQSGGGLVQAGGSLRLTCTASGRTVSNYAMGWFRQTPGKDREFVAALNWSGDTTSYADSV
RGRFTISRDNTRNTVYLQMDSLKREDTAVYYCAAAQSFRRGGAPYGDNYWGQGTQVTVSS
CDR1:
(SEQ ID NO: 134)
SGRTVSNYAM
CDR2:
(SEQ ID NO: 135)
ALNWGGDTTS
CDR3:
(SEQ ID NO: 136)
AAAQSFRRGGAPYGDNYW
hz2C6v1
(SEQ ID NO: 22)
EVQLLESGGGLVQPGGSLRLSCAASGRTVSNYAMSWFRQAPGKGLEFVSALNWGGDTTYYADSV
KGRFTISRDNSKNTLYLQMNSLRAEDTAVYYCAAAQSFRRGGAPYGDNYWWGQGTLVTVSS
CDR1:
(SEQ ID NO: 134)
SGRTVSNYAM
CDR2:
(SEQ ID NO: 137)
ALNWGGDTTY
CDR3:
(SEQ ID NO: 136)
AAAQSFRRGGAPYGDNYW
hz2C6v1opt
(SEQ ID NO: 23)
EVQLLESGGGEVQPGGSLRLSCAASGRTVSNYAMSWFRQAPGKGLEFVSALNWGGDTTYYAESV
KGRFTISRDNAKNTLYLQMSSLRAEDTAVYYCAAAQSFRRGGAPYGDNYWGQGTLVTVKPGG
CDR1:
(SEQ ID NO: 134)
SGRTVSNYAM
CDR2:
(SEQ ID NO: 137)
ALNWGGDTTY
CDR3:
(SEQ ID NO: 136)
AAAQSFRRGGAPYGDNYW
hzC06v2
(SEQ ID NO: 91)
EVQLLESGGGEVQPGGSLRLSCAASGRTVSNYAMGWFRQAPGKDREFVSALNWGGDTTYYAESV
KGRFTISRDNAKNTLYLQMSSLRAEDTAVYYCAAAQSFRRGGAPYGDNYWGQGTLVTVKP
CDR1:
(SEQ ID NO: 134)
SGRTVSNYAM
CDR2:
(SEQ ID NO: 137)
ALNWGGDTTY
CDR3:
(SEQ ID NO: 136)
AAAQSFRRGGAPYGDNYW
C12
(SEQ ID NO: 24)
EVQLVQSGGGLVQAGDSLRLSCAASGRALTGYHMAWFRQAPGKEREFVTYGIWDRAGAAYADSV
KGRFTMSRDNAKNTVYLQMNNLKTEDTAVYYCAASMAVRTYYSPRSYDSWGQGTQVTVSS
CDR1:
(SEQ ID NO: 138)
SGRALTGYHMAW
CDR2:
(SEQ ID NO: 139)
YGIWDRAGAA
CDR3:
(SEQ ID NO: 140)
ASMAVRTYYSPRSYDSW
hzC12v2
(SEQ ID NO: 25)
EVQLLESGGGLVQPGGSLRLSCAASGRALTGYHMSWFRQAPGKGREFVSYGIWDRAGAAYADSV
KGRFTISRDNSKNTLYLQMNSLRAEDTAVYYCAASMAVRTYYSPRSYDSWGQGTLVTVSS
CDR1:
(SEQ ID NO: 141)
SGRALTGYHMSW
CDR2:
(SEQ ID NO: 139)
YGIWDRAGAA
CDR3:
(SEQ ID NO: 140)
ASMAVRTYYSPRSYDSW
hzC12v3
(SEQ ID NO: 26)
EVQLLESGGGLVQPGGSLRLSCAASGRALTGYHMSWFRQAPGKGLEFVSYGIWDRAGAAYADSV
KGRFTISRDNSKNTLYLQMNSLRAEDTAVYYCAASMAVRTYYSPRSYDSWGQGTLVTVSS
CDR1:
(SEQ ID NO: 141)
SGRALTGYHMSW
CDR2:
(SEQ ID NO: 139)
YGIWDRAGAA
CDR3:
(SEQ ID NO: 140)
ASMAVRTYYSPRSYDSW
1F2
(SEQ ID NO: 27)
EVQLVQSGGGLVQAGGSLRLSCAASGSTFSSLDMGWFRQAPGKERAFVAAISRSGDNIYYAESV
KGRFTISRDNAENTTYLQMNSLKPEDSAVYYCAVDSQPTYSGGVYYPRYGMDVWGQGTQVTVSS
CDR1:
(SEQ ID NO: 142)
SGSTFSSLDMGW
CDR2:
(SEQ ID NO: 143)
AISRSGDNIY
CDR3:
(SEQ ID NO: 144)
AVDSQPTYSGGVYYPRYGMDVW
hz1F2v2
(SEQ ID NO: 29)
EVQLLESGGGLVQPGGSLRLSCAASGSTFSSLDMGWFRQAPGKGREFVSAISRSGDNIYYADSV
KGRFTISRDNSKNTLYLQMNSLRAEDTAVYYCAVDSQPTYSGGVYYPRYGMDVWGQGTLVTVSS
CDR1:
(SEQ ID NO: 142)
SGSTFSSLDMGW
CDR2:
(SEQ ID NO: 143)
AISRSGDNIY
CDR3:
(SEQ ID NO: 144)
AVDSQPTYSGGVYYPRYGMDVW
hz1F2v1
(SEQ ID NO: 30)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDSQPTYSGGVYYPRYGMDVWGQGTLVTVKP
CDR1:
(SEQ ID NO: 142)
SGSTFSSLDMGW
CDR2:
(SEQ ID NO: 143)
AISRSGDNIY
CDR3:
(SEQ ID NO: 144)
AVDSQPTYSGGVYYPRYGMDVW
hz1F2v2
(SEQ ID NO: 32)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDTQPTYSGGVYYPRYGMDVWGQGTLVTVKP
CDR1:
(SEQ ID NO: 142)
SGSTFSSLDMGW
CDR2:
(SEQ ID NO: 143)
AISRSGDNIY
CDR3:
(SEQ ID NO: 145)
AVDTQPTYSGGVYYPRYGMDVW
hz1F2v3
(SEQ ID NO: 33)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDAQPTYSGGVYYPRYGMDVWGQGTLVTVKP
CDR1:
(SEQ ID NO: 142)
SGSTFSSLDMGW
CDR2:
(SEQ ID NO: 143)
AISRSGDNIY
CDR3:
(SEQ ID NO: 146)
AVDAQPTYSGGVYYPRYGMDVW
hz1F2v4
(SEQ ID NO: 34)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVESQPTYSGGVYYPRYGMDVWGQGTLVTVKP
CDR1:
(SEQ ID NO: 142)
SGSTFSSLDMGW
CDR2:
(SEQ ID NO: 143)
AISRSGDNIY
CDR3:
(SEQ ID NO: 147)
AVESQPTYSGGVYYPRYGMDVW
hz1F2v5
(SEQ ID NO: 35)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDSQPTYSGGVYYPRYGYDVWGQGTLVTVKP
GG
CDR1:
(SEQ ID NO: 142)
SGSTFSSLDMGW
CDR2:
(SEQ ID NO: 143)
AISRSGDNIY
CDR3:
(SEQ ID NO: 148)
AVDSQPTYSGGVYYPRYGYDVW
hz1F2v6
(SEQ ID NO: 36)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDSQPTYSGGVYYPRYGDDVWGQGTLVTVKP
GG
CDR1:
(SEQ ID NO: 142)
SGSTFSSLDMGW
CDR2:
(SEQ ID NO: 143)
AISRSGDNIY
CDR3:
(SEQ ID NO: 148)
AVDSQPTYSGGVYYPRYGDDVW
hz1F2v7
(SEQ ID NO: 37)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDSQPTYSGGVYYPRYGLDVWGQGTLVTVKP
GG
CDR1:
(SEQ ID NO: 142)
SGSTFSSLDMGW
CDR2:
(SEQ ID NO: 143)
AISRSGDNIY
CDR3:
(SEQ ID NO: 149)
AVDSQPTYSGGVYYPRYGLDVW
hz1F2-DS
(SEQ ID NO: 38)
EVQLLESGGGEVQPGGSLRLSCAASGSTFSSLDMGWFRQAPGKGREFVCAISRSGDNIYYAESV
KGRFTCSRDNAKNTLYLQMSSLRAEDTAVYYCAVESQPTYSGGVYYPRYGMDVWGQGTLVTVKP
GG
CDR1:
(SEQ ID NO: 142)
SGSTFSSLDMGW
CDR2:
(SEQ ID NO: 143)
AISRSGDNIY
CDR3:
(SEQ ID NO: 147)
AVESQPTYSGGVYYPRYGMDVW
hz1F2-MA
(SEQ ID NO: 39)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDAQPTYSGGVYYPRYGADVWGQGTLVTVKP
GG
CDR1:
(SEQ ID NO: 142)
SGSTFSSLDMGW
CDR2:
(SEQ ID NO: 143)
AISRSGDNIY
CDR3:
(SEQ ID NO: 150)
AVDAQPTYSGGVYYPRYGADVW
hz1F2-ME
(SEQ ID NO: 40)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDAQPTYSGGVYYPRYGEDVWGQGTLVTVKP
GG
CDR1:
(SEQ ID NO: 142)
SGSTFSSLDMGW
CDR2:
(SEQ ID NO: 143)
AISRSGDNIY
CDR3:
(SEQ ID NO: 150)
AVDAQPTYSGGVYYPRYGEDVW
hz1F2-MH
(SEQ ID NO: 41)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDAQPTYSGGVYYPRYGHDVWGQGTLVTVKP
GG
CDR1:
(SEQ ID NO: 142)
SGSTFSSLDMGW
CDR2:
(SEQ ID NO: 143)
AISRSGDNIY
CDR3:
(SEQ ID NO: 151)
AVDAQPTYSGGVYYPRYGHDVW
hz1F2-MN
(SEQ ID NO: 42)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDAQPTYSGGVYYPRYGNDVWGQGTLVTVKP
GG
CDR1:
(SEQ ID NO: 142)
SGSTFSSLDMGW
CDR2:
(SEQ ID NO: 143)
AISRSGDNIY
CDR3:
(SEQ ID NO: 152)
AVDAQPTYSGGVYYPRYGNDVW
hz1F2-MP
(SEQ ID NO: 43)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDAQPTYSGGVYYPRYGPDVWGQGTLVTVKP
GG
CDR1:
(SEQ ID NO: 142)
SGSTFSSLDMGW
CDR2:
(SEQ ID NO: 143)
AISRSGDNIY
CDR3:
(SEQ ID NO: 153)
AVDAQPTYSGGVYYPRYGPDVW
hz1F2-MQ
(SEQ ID NO: 44)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDAQPTYSGGVYYPRYGQDVWGQGTLVTVKP
GG
CDR1:
(SEQ ID NO: 142)
SGSTFSSLDMGW
CDR2:
(SEQ ID NO: 143)
AISRSGDNIY
CDR3:
(SEQ ID NO: 154)
AVDAQPTYSGGVYYPRYGQDVW
hz1F2-MR
(SEQ ID NO: 45)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDAQPTYSGGVYYPRYGRDVWGQGTLVTVKP
GG
CDR1:
(SEQ ID NO: 142)
SGSTFSSLDMGW
CDR2:
(SEQ ID NO: 143)
AISRSGDNIY
CDR3:
(SEQ ID NO: 155)
AVDAQPTYSGGVYYPRYGRDVW
hz1F2-MS
(SEQ ID NO: 46)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDAQPTYSGGVYYPRYGSDVWGQGTLVTVKP
GG
CDR1:
(SEQ ID NO: 142)
SGSTFSSLDMGW
CDR2:
(SEQ ID NO: 143)
AISRSGDNIY
CDR3:
(SEQ ID NO: 156)
AVDAQPTYSGGVYYPRYGSDVW
hz1F2-MT
(SEQ ID NO: 47)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDAQPTYSGGVYYPRYGTDVWGQGTLVTVKP
GG
CDR1:
(SEQ ID NO: 142)
SGSTFSSLDMGW
CDR2:
(SEQ ID NO: 143)
AISRSGDNIY
CDR3:
(SEQ ID NO: 157)
AVDAQPTYSGGVYYPRYGTDVW
hz1F2-MV
(SEQ ID NO: 48)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVDAQPTYSGGVYYPRYGVDVWGQGTLVTVKP
GG
CDR1:
(SEQ ID NO: 142)
SGSTFSSLDMGW
CDR2:
(SEQ ID NO: 143)
AISRSGDNIY
CDR3:
(SEQ ID NO: 158)
AVDAQPTYSGGVYYPRYGVDVW
B04
(SEQ ID NO: 49)
EVQLVQSGGGLVQAGGSLRLSCAASGRAFSNYALGWFRQAPGKEREFIAAINWNGENRYGVDSV
KGRFTISRDNAQNMGYLQMNNLKPEDTAVYRCAAALSFRIGGEPYGDAYWGQGTQVTVSS
CDR1:
(SEQ ID NO: 159)
SGRAFSNYALGW
CDR2:
(SEQ ID NO: 160)
AINWNGENRY
CDR3:
(SEQ ID NO: 161)
AAALSFRIGGEPYGDAYW
hzB04v1
(SEQ ID NO: 50)
EVQLLESGGGLVQPGGSLRLSCAASGRAFSNYAMSWFRQAPGKGLEFVSAINWNGENRYYADSV
KGRFTISRDNSKNTLYLQMNSLRAEDTAVYYCAAALSFRIGGEPYGDAYWGQGTLVTVSS
CDR1:
(SEQ ID NO: 162)
SGRAFSNYAMSW
CDR2:
(SEQ ID NO: 160)
AINWNGENRY
CDR3:
(SEQ ID NO: 161)
AAALSFRIGGEPYGDAYW
5A04
(SEQ ID NO: 51)
QVQLQESGGGLVQAGGSLRLSCVASGSIFTNNAMGWYRQAPGKQRDLVAQITMGGGITNYAPSM
EGRFAISRDNAKSTVYLQMNNLKPEDTAVYYCNAEVKSADWGAYANYWGQGTQVTVSS
CDR1:
(SEQ ID NO: 163)
SGSIFTNNAM
CDR2:
(SEQ ID NO: 164)
QITMGGGITN
CDR3:
(SEQ ID NO: 165)
NAEVKSADWGAYANYW
hz5A04v1
(SEQ ID NO: 52)
EVQLLESGGGLVQPGGSLRLSCAASGSIFTNNAMSWYRQAPGKGLELVSAITMGGGITYYADSV
KGRFTISRDNSKNTLYLQMNSLRAEDTAVYYCNAEVKSADWGAYANYWGQGTLVTVSS
CDR1:
(SEQ ID NO: 163)
SGSIFTNNAM
CDR2:
(SEQ ID NO: 166)
AITMGGGITY
CDR3:
(SEQ ID NO: 165)
NAEVKSADWGAYANYW
hz5A04v2
(SEQ ID NO: 53)
EVQLLESGGGLVQPGGSLRLSCAASGSIFTNNAMSWYRQAPGKGRELVSQITMGGGITYYADSV
KGRFTISRDNSKNTLYLQMNSLRAEDTAVYYCNAEVKSADWGAYANYWGQGTLVTVSS
CDR1:
(SEQ ID NO: 163)
SGSIFTNNAM
CDR2:
(SEQ ID NO: 167)
QITMGGGITY
CDR3:
(SEQ ID NO: 165)
NAEVKSADWGAYANYW
F03
(SEQ ID NO: 54)
QVQLQESGGGLVQAGGSLRLSCAASGRSISNYAMGWFRQAPGKEREFLAASVWNNGGNYYADSV
KGRFTASRDDAKSTAYLQMSRLRPEDTGIYYCVVARTPETPITSARGANYWGQGTQVTVSS
CDR1:
(SEQ ID NO: 168)
SGRSISNYAM
CDR2:
(SEQ ID NO: 169)
ASVWNNGGNY
CDR3:
(SEQ ID NO: 170)
VVARTPETPITSARGANYW
hzF03v2
(SEQ ID NO: 55)
EVQLLESGGGLVQPGGSLRLSCAASGRSISNYAMGWFRQAPGKEREFVSASVWNNGGNYYADSV
KGRFTISRDNSKNTLYLQMNSLRAEDTAVYYCVVARTPETPITSARGANYWGQGTLVTVSS
CDR1:
(SEQ ID NO: 168)
SGRSISNYAM
CDR2:
(SEQ ID NO: 169)
ASVWNNGGNY
CDR3:
(SEQ ID NO: 170)
VVARTPETPITSARGANYW
hzF03v1opt
(SEQ ID NO: 56)
EVQLLESGGGEVQPGGSLRLSCAASGRSISNYAMGWFRQAPGKEREFVSASVWNNGGNYYAESV
KGRFTISRDNAKNTLYLQMSSLRAEDTAVYYCVVARTPETPITSARGANYWGQGTLVTVKPGG
CDR1:
(SEQ ID NO: 168)
SGRSISNYAM
CDR2:
(SEQ ID NO: 169)
ASVWNNGGNY
CDR3:
(SEQ ID NO: 170)
VVARTPETPITSARGANYW
hzF03v2opt
(SEQ ID NO: 57)
EVQLLESGGGEVQPGGSLRLSCAASGRSISNYAMGWFRQAPGKEREFVSASVWNNGGNYYAESV
KGRFTISRDDAKSTLYLQMSSLRAEDTAVYYCVVARTPETPITSARGANYWGQGTLVTVKPGG
CDR1:
(SEQ ID NO: 168)
SGRSISNYAM
CDR2:
(SEQ ID NO: 169)
ASVWNNGGNY
CDR3:
(SEQ ID NO: 170)
VVARTPETPITSARGANYW
hzF03v3opt
(SEQ ID NO: 58)
EVQLLESGGGEVQPGGSLRLSCAASGRSISNYAMGWFRQAPGKEREFVSASVWNQGGNYYAESV
KGRFTISRDNAKNTLYLQMSSLRAEDTAVYYCVVARTPETPITSARGANYWGQGTLVTVKPGG
CDR1:
(SEQ ID NO: 168)
SGRSISNYAM
CDR2:
(SEQ ID NO: 171)
ASVWNQGGNY
CDR3:
(SEQ ID NO: 170)
VVARTPETPITSARGANYW
hzF03v4opt
(SEQ ID NO: 59)
EVQLLESGGGEVQPGGSLRLSCAASGRSISNYAMGWFRQAPGKEREFVSASVWNNAGNYYAESV
KGRFTISRDNAKNTLYLQMSSLRAEDTAVYYCVVARTPETPITSARGANYWGQGTLVTVKPGG
CDR1:
(SEQ ID NO: 168)
SGRSISNYAM
CDR2:
(SEQ ID NO: 172)
ASVWNNAGNY
CDR3:
(SEQ ID NO: 170)
VVARTPETPITSARGANYW
hzF03v5opt
(SEQ ID NO: 60)
EVQLLESGGGEVQPGGSLRLSCAASGRSISNYAMGWFRQAPGKEREFVSASVWNQGGNYYAESV
KGRFTISRDDAKSTLYLQMSSLRAEDTAVYYCVVARTPETPITSARGANYWGQGTLVTVKPGG
CDR1:
(SEQ ID NO: 168)
SGRSISNYAM
CDR2:
(SEQ ID NO: 171)
ASVWNQGGNY
CDR3:
(SEQ ID NO: 170)
VVARTPETPITSARGANYW
hzF03v6opt
(SEQ ID NO: 61)
EVQLLESGGGEVQPGGSLRLSCAASGRSISNYAMGWFRQAPGKEREFVSASVWNNAGNYYAESV
KGRFTISRDDAKSTLYLQMSSLRAEDTAVYYCVVARTPETPITSARGANYWGQGTLVTVKPGG
CDR1:
(SEQ ID NO: 168)
SGRSISNYAM
CDR2:
(SEQ ID NO: 172)
ASVWNNAGNY
CDR3:
(SEQ ID NO: 170)
VVARTPETPITSARGANYW
3B7
(SEQ ID NO: 62)
QVQLQESGGGSVQAGGSLTLSCAASGRAASDYAVGWFRQAPGKEREFVAACNWSGEDTVYAYIV
KGRFTISRDNAGNTVSLRMSSLEPEDTAVYYCAAAPSFSRSVLDGNLSQIDYWGQGTQVTVSS
CDR1:
(SEQ ID NO: 173)
SGRAASDYAV
CDR2:
(SEQ ID NO: 174)
ACNWSGEDTV
CDR3:
(SEQ ID NO: 175)
AAAPSFSRSVLDGNLSQIDYW
hz3B7v2
(SEQ ID NO: 63)
EVQLLESGGGLVQPGGSLRLSCAASGRAASDYAMSWFRQAPGKGLEFVSAINWGGEDTVYADSV
KGRFTISRDNSKNTLYLQMNSLRAEDTAVYYCAAAPSFSRSVLDGNLSQIDYWGQGTLVTVSS
CDR1:
(SEQ ID NO: 176)
SGRAASDYAM
CDR2:
(SEQ ID NO: 177)
INWGGEDTV
CDR3:
(SEQ ID NO: 175)
AAAPSESRSVLDGNLSQIDYW
6G01
(SEQ ID NO: 64)
QVQLVQSGGGLAQAGGSLRLSCVASGRTFTNYAMGWFRQAPGKEREFVAAINWSGDSTYHADSV
KGRFTISRDNAKDSVYLQMTKLKPEDTADYYCASAESFSRGGLPYGMNYWGQGTQVTVSS
CDR1:
(SEQ ID NO: 178)
SGRTFTNYAM
CDR2:
(SEQ ID NO: 179)
AINWSGDSTY
CDR3:
(SEQ ID NO: 180)
ASAESFSRGGLPYGMNYW
hz6G01v1
(SEQ ID NO: 65)
EVQLLESGGGLVQPGGSLRLSCAASGRTFTNYAMSWFRQAPGKGLEFVSAINWSGDSTYYADSV
KGRFTISRDNSKNTLYLQMNSLRAEDTAVYYCASAESFSRGGLPYGMNYWGQGTLVTVSS
CDR1:
(SEQ ID NO: 178)
SGRTFTNYAM
CDR2:
(SEQ ID NO: 179)
AINWSGDSTY
CDR3:
(SEQ ID NO: 180)
ASAESFSRGGLPYGMNYW
hz6G01v1opt
(SEQ ID NO: 66)
EVQLLESGGGEVQPGGSLRLSCAASGRTFTNYAMSWFRQAPGKGLEFVSAINWSGDSTYYAESV
KGRFTISRDNAKNTLYLQMSSLRAEDTAVYYCASAESFSRGGLPYGMNYWGQGTLVTVKPGG
CDR1:
(SEQ ID NO: 178)
SGRTFTNYAM
CDR2:
(SEQ ID NO: 179)
AINWSGDSTY
CDR3:
(SEQ ID NO: 180)
ASAESFSRGGLPYGMNYW
H10
(SEQ ID NO: 67)
QVQLVQSGGGLVQAGGSLTLSCAASVSTFGTSPVGWFRQAPGKEREFVSAIRWDGVGAYYADSV
RGRFKNSKDNAKRTAYLQMNRLKPEDTAVYYCALPRRGDSELPSTVKEYGYWGQGTQVTVSS
CDR1:
(SEQ ID NO: 181)
SVSTFGTSPV
CDR2:
(SEQ ID NO: 182)
AIRWDGVGAY
CDR3:
(SEQ ID NO: 183)
ALPRRGDSELPSTVKEYGYW
hzH10v3
(SEQ ID NO: 68)
EVQLLESGGGEVQPGGSLRLSCAASVSTFGTSPVGWFRQAPGKEREFVSAIRWEGVGAYYAESV
KGRFTISRDNAKNTLYLQMSSLRAEDTAVYYCALPRRGDSELPSTVKEYGYWGQGTLVTVKP
CDR1:
(SEQ ID NO: 181)
SVSTFGTSPV
CDR2:
(SEQ ID NO: 184)
AIRWEGVGAY
CDR3:
(SEQ ID NO: 183)
ALPRRGDSELPSTVKEYGYW
hzH10v2
(SEQ ID NO: 69)
EVQLLESGGGLVQPGGSLRLSCAASVSTFGTSPVGWFRQAPGKEREFVSAIRWDGVGAYYADSV
KGRFTISRDNSKNTLYLQMNSLRAEDTAVYYCALPRRGDSELPSTVKEYGYWGQGTLVTVSS
CDR1:
(SEQ ID NO: 181)
SVSTFGTSPV
CDR2:
(SEQ ID NO: 182)
AIRWDGVGAY
CDR3:
(SEQ ID NO: 183)
ALPRRGDSELPSTVKEYGYW
hzH10v1opt
(SEQ ID NO: 70)
EVQLLESGGGEVQPGGSLRLSCAASVSTFGTSPVGWFRQAPGKEREFVSAIRWDGVGAYYAESV
KGRFTISRDNAKNTLYLQMSSLRAEDTAVYYCALPRRGDSELPSTVKEYGYWGQGTLVTVKPGG
CDR1:
(SEQ ID NO: 181)
SVSTFGTSPV
CDR2:
(SEQ ID NO: 182)
AIRWDGVGAY
CDR3:
(SEQ ID NO: 183)
ALPRRGDSELPSTVKEYGYW
hzH10-DS
(SEQ ID NO: 71)
EVQLLESGGGEVQPGGSLRLSCAASVSTFGTSPVGWFRQAPGKEREFVCAIRWEGVGAYYAESV
KGRFTCSRDNAKNTLYLQMSSLRAEDTAVYYCALPRRGDSELPSTVKEYGYWGQGTLVTVKPGG
CDR1:
(SEQ ID NO: 181)
SVSTFGTSPV
CDR2:
(SEQ ID NO: 184)
AIRWEGVGAY
CDR3:
(SEQ ID NO: 183)
ALPRRGDSELPSTVKEYGYW
hzH10v4opt
(SEQ ID NO: 72)
EVQLLESGGGEVQPGGSLRLSCAASVSTFGTSPVGWFRQAPGKEREFVSAIRWDAVGAYYAESV
KGRFTISKDNAKRTLYLQMSSLRAEDTAVYYCALPRRGDSELPSTVKEYGYWGQGTLVTVKPGG
CDR1:
(SEQ ID NO: 181)
SVSTFGTSPV
CDR2:
(SEQ ID NO: 185)
AIRWDAVGAY
CDR3:
(SEQ ID NO: 183)
ALPRRGDSELPSTVKEYGYW
hzH10v5opt
(SEQ ID NO: 73)
EVQLLESGGGEVQPGGSLRLSCAASVSTFGTSPVGWFRQAPGKEREFVSAIRWDGVGAYYAESV
KGRFTISKDNAKRTLYLQMSSLRAEDTAVYYCALPRRGESELPSTVKEYGYWGQGTLVTVKPGG
CDR1:
(SEQ ID NO: 181)
SVSTFGTSPV
CDR2:
(SEQ ID NO: 182)
AIRWDGVGAY
CDR3:
(SEQ ID NO: 186)
ALPRRGESELPSTVKEYGYW
hzH10v6opt
(SEQ ID NO: 74)
EVQLLESGGGEVQPGGSLRLSCAASVSTFGTSPVGWFRQAPGKEREFVSAIRWDGVGAYYAESV
KGRFTISKDNAKRTLYLQMSSLRAEDTAVYYCALPRRGDAELPSTVKEYGYWGQGTLVTVKPGG
CDR1:
(SEQ ID NO: 181)
SVSTFGTSPV
CDR2:
(SEQ ID NO: 182)
AIRWDGVGAY
CDR3:
(SEQ ID NO: 187)
ALPRRGDAELPSTVKEYGYW
hzH10v7opt
(SEQ ID NO: 75)
EVQLLESGGGEVQPGGSLRLSCAASVSTFGTSPVGWFRQAPGKEREFVSAIRWDGVGAYYAESV
KGRFTISRDNAKNTLYLQMSSLRAEDTAVYYCALPRRGDSELPSTVKEYGYWGQGTLVTVKPGG
CDR1:
(SEQ ID NO: 181)
SVSTFGTSPV
CDR2:
(SEQ ID NO: 182)
AIRWDGVGAY
CDR3:
(SEQ ID NO: 183)
ALPRRGDSELPSTVKEYGYW
hzH10v8opt
(SEQ ID NO: 76)
EVQLLESGGGEVQPGGSLRLSCAASVSTFGTSPVGWFRQAPGKEREFVSAIRWEGVGAYYAESV
KGRFTISKDNAKRTLYLQMSSLRAEDTAVYYCALPRRGESELPSTVKEYGYWGQGTLVTVKPGG
CDR1:
(SEQ ID NO: 181)
SVSTFGTSPV
CDR2:
(SEQ ID NO: 184)
AIRWEGVGAY
CDR3:
(SEQ ID NO: 186)
ALPRRGESELPSTVKEYGYW
hzH10opt
(SEQ ID NO: 77)
EVQLLESGGGEVQPGGSLRLSCAASVSTFGTSPVGWFRQAPGKEREFVSAIRWEGVGAYYAESV
KGRFTISKDNAKRTLYLQMSSLRAEDTAVYYCALPRRGDAELPSTVKEYGYWGQGTLVTVKPGG
CDR1:
(SEQ ID NO: 181)
SVSTFGTSPV
CDR2:
(SEQ ID NO: 184)
AIRWEGVGAY
CDR3:
(SEQ ID NO: 187)
ALPRRGDAELPSTVKEYGYW
hzH10v10opt
(SEQ ID NO: 78)
EVQLLESGGGEVQPGGSLRLSCAASVSTFGTSPVGWFRQAPGKEREFVSAIRWDAVGAYYAESV
KGRFTISKDNAKRTLYLQMSSLRAEDTAVYYCALPRRGESELPSTVKEYGYWGQGTLVTVKPGG
CDR1:
(SEQ ID NO: 181)
SVSTFGTSPV
CDR2:
(SEQ ID NO: 185)
AIRWDAVGAY
CDR3:
(SEQ ID NO: 186)
ALPRRGESELPSTVKEYGYW
H11
(SEQ ID NO: 79)
QLQLQESGGGLVQAGDSLRLSCQVSGRTLSAYLMAWFRQAPNKVREYLGRIRWNEGDTYYPDSV
KGRFTISKDDAKNTVYLRMNSLKPEDTAVYYCAARSIFNPSDQYVYWGQGTQVTVSS
CDR1:
(SEQ ID NO: 188)
SGRTLSAYLM
CDR2:
(SEQ ID NO: 189)
RIRWNEGDTY
CDR3:
(SEQ ID NO: 190)
AARSIFNPSDQYVYW
hzH11v1
(SEQ ID NO: 80)
EVQLLESGGGLVQPGGSLRLSCAASGRTLSAYLMSWFRQAPGKGLEYVSAIRWNEGDTYYADSV
KGRFTISRDNSKNTLYLQMNSLRAEDTAVYYCAARSIFNPSDQYVYWGQGTLVTVSS
CDR1:
(SEQ ID NO: 188)
SGRTLSAYLM
CDR2:
(SEQ ID NO: 28)
AIRWNEGDTY
CDR3:
(SEQ ID NO: 190)
AARSIFNPSDQYVYW
hzH11v2
(SEQ ID NO: 81)
EVQLLESGGGLVQPGGSLRLSCAVSGRTLSAYLMSWFRQAPGKGREYVSRIRWNEGDTYYADSV
KGRFTISRDNSKNTLYLQMNSLKAEDTAVYYCAARSIFNPSDQYVYWGQGTLVTVSS
CDR1:
(SEQ ID NO: 188)
SGRTLSAYLM
CDR2:
(SEQ ID NO: 189)
RIRWNEGDTY
CDR3:
(SEQ ID NO: 190)
AARSIFNPSDQYVYW
1F10
(SEQ ID NO: 82)
EVQLVQSGGGLVQAGGSLRLSCAASGSTFSSLDMGWFRQAPGKERAFVAAISRSGDNIYYAESV
KGRFTISRDNAENTMYLQMNSLKPEDSAVYYCAVESQPTYSGGVYYPRYGMDVWGQGTQVTVSS
CDR1:
(SEQ ID NO: 142)
SGSTFSSLDMGW
CDR2:
(SEQ ID NO: 143)
AISRSGDNIY
CDR3:
(SEQ ID NO: 147)
AVESQPTYSGGVYYPRYGMDVW
hz1F10
(SEQ ID NO: 83)
EVQLLESGGGLVQPGGSLRLSCAASGSTFSSLDMSWFRQAPGKGLEFVSAISRSGDNIYYADSV
KGRFTISRDNSKNTLYLQMNSLRAEDTAVYYCAVESQPTYSGGVYYPRYGMDVWGQGTLVTVSS
CDR1:
(SEQ ID NO: 31)
SGSTFSSLDMSW
CDR2:
(SEQ ID NO: 143)
AISRSGDNIY
CDR3:
(SEQ ID NO: 147)
AVESQPTYSGGVYYPRYGMDVW
hz1F10v2
(SEQ ID NO: 84)
EVQLLESGGGLVQPGGSLRLSCAASGSTFSSLDMGWFRQAPGKGREFVSAISRSGDNIYYADSV
KGRFTISRDNSKNTLYLQMNSLRAEDTAVYYCAVESQPTYSGGVYYPRYGMDVWGQGTLVTVSS
CDR1:
(SEQ ID NO: 142)
SGSTFSSLDMGW
CDR2:
(SEQ ID NO: 143)
AISRSGDNIY
CDR3:
(SEQ ID NO: 147)
AVESQPTYSGGVYYPRYGMDVW
2x_1F5-DS
(SEQ ID NO: 92)
EVQLLESGGGEVQPGGSLRLSCAASGLTFPNYGMSWFRQAPGKGLEFVCAIYWSGGTVYYAESV
KGRFTCSRDNAKNTLYLQMSSLRAEDTAVYYCAVTIRGAATQTWKYDYWGQGTLVTVKPGGSGG
SEVQLLESGGGEVQPGGSLRLSCAASGLTFPNYGMSWFRQAPGKGLEFVCAIYWSGGTVYYAES
VKGRFTCSRDNAKNTLYLQMSSLRAEDTAVYYCAVTIRGAATQTWKYDYWGQGTLVTVKPGGGG
2x_1F5
(SEQ ID NO: 93)
EVQLLESGGGEVQPGGSLRLSCAASGLTFPNYGMSWFRQAPGKGLEFVSAIYWSGGTVYYAESV
KGRFTISRDNAKNTLYLQMSSLRAEDTAVYYCAVTIRGAATQTWKYDYWGQGTLVTVKPGGSGG
SEVQLLESGGGEVQPGGSLRLSCAASGLTFPNYGMSWFRQAPGKGLEFVSAIYWSGGTVYYAES
VKGRFTISRDNAKNTLYLQMSSLRAEDTAVYYCAVTIRGAATQTWKYDYWGQGTLVTVKPGGGG
2x_1F5_gs6
(SEQ ID NO: 94)
EVQLLESGGGLVQPGGSLRLSCAASGSTFSSLDMGWFRQAPGKGREFVSAISRSGDNIYYADSV
KGRFTISRDNSKNTLYLQMNSLRAEDTAVYYCAVDSQPTYSGGVYYPRYGMDVWGQGTLVTVSG
SGGGGSEVQLLESGGGLVQPGGSLRLSCAASGSTFSSLDMGWFRQAPGKGREFVSAISRSGDNI
YYADSVKGRFTISRDNSKNTLYLQMNSLRAEDTAVYYCAVDSQPTYSGGVYYPRYGMDVWGQGT
LVTVSSAGGGG
2x_1F5_gs12
(SEQ ID NO: 95)
EVQLLESGGGLVQPGGSLRLSCAASGLTFPNYGMSWFRQAPGKGLEFVSAIYWSGGTVYYADSV
KGRFTISRDNSKNTLYLQMNSLRAEDTAVYYCAVTIRGAATQTWKYDYWGQGTLVTVSSGGGSG
GSGGGGSEVQLLESGGGLVQPGGSLRLSCAASGLTFPNYGMSWFRQAPGKGLEFVSAIYWSGGT
VYYADSVKGRFTISRDNSKNTLYLQMNSLRAEDTAVYYCAVTIRGAATQTWKYDYWGQGTLVTV
SSAGGGG
2x_1F5_gs15
(SEQ ID NO: 96)
EVQLLESGGGLVQPGGSLRLSCAASGLTFPNYGMSWFRQAPGKGLEFVSAIYWSGGTVYYADSV
KGRFTISRDNSKNTLYLQMNSLRAEDTAVYYCAVTIRGAATQTWKYDYWGQGTLVTVSSGGGGS
GGGGSGGGGSEVQLLESGGGLVQPGGSLRLSCAASGLTFPNYGMSWFRQAPGKGLEFVSAIYWS
GGTVYYADSVKGRFTISRDNSKNTLYLQMNSLRAEDTAVYYCAVTIRGAATQTWKYDYWGQGTL
VTVSSAGGGG
2x_hz1F2v2-gs6
(SEQ ID NO: 97)
EVQLLESGGGLVQPGGSLRLSCAASGSTFSSLDMGWFRQAPGKGREFVSAISRSGDNIYYADSV
KGRFTISRDNSKNTLYLQMNSLRAEDTAVYYCAVDSQPTYSGGVYYPRYGMDVWGQGTLVTVSG
SGGGGSEVQLLESGGGLVQPGGSLRLSCAASGSTFSSLDMGWFRQAPGKGREFVSAISRSGDNI
YYADSVKGRFTISRDNSKNTLYLQMNSLRAEDTAVYYCAVDSQPTYSGGVYYPRYGMDVWGQGT
LVTVSSAGGGG
2x_hz1F2v2-gs9
(SEQ ID NO: 98)
EVQLLESGGGLVQPGGSLRLSCAASGSTFSSLDMGWFRQAPGKGREFVSAISRSGDNIYYADSV
KGRFTISRDNSKNTLYLQMNSLRAEDTAVYYCAVDSQPTYSGGVYYPRYGMDVWGQGTLVTVSS
GGGSGGGGSEVQLLESGGGLVQPGGSLRLSCAASGSTFSSLDMGWFRQAPGKGREFVSAISRSG
DNIYYADSVKGRFTISRDNSKNTLYLQMNSLRAEDTAVYYCAVDSQPTYSGGVYYPRYGMDVWG
QGTLVTVSSAGGGG
2x_hz1F2v2-gs12
(SEQ ID NO: 99)
EVQLLESGGGLVQPGGSLRLSCAASGSTFSSLDMGWFRQAPGKGREFVSAISRSGDNIYYADSV
KGRFTISRDNSKNTLYLQMNSLRAEDTAVYYCAVDSQPTYSGGVYYPRYGMDVWGQGTLVTVSS
GGGSGGSGGGGSEVQLLESGGGLVQPGGSLRLSCAASGSTFSSLDMGWFRQAPGKGREFVSAIS
RSGDNIYYADSVKGRFTISRDNSKNTLYLQMNSLRAEDTAVYYCAVDSQPTYSGGVYYPRYGMD
VWGQGTLVTVSSAGGGG
2x_hz1F2v2-gs15
(SEQ ID NO: 100)
EVQLLESGGGLVQPGGSLRLSCAASGSTFSSLDMSWFRQAPGKGLEFVSAISRSGDNIYYADSV
KGRFTISRDNSKNTLYLQMNSLRAEDTAVYYCAVDSQPTYSGGVYYPRYGMDVWGQGTLVTVSS
GGGGSGGGGSGGGGSEVQLLESGGGLVQPGGSLRLSCAASGSTFSSLDMSWFRQAPGKGLEFVS
AISRSGDNIYYADSVKGRFTISRDNSKNTLYLQMNSLRAEDTAVYYCAVDSQPTYSGGVYYPRY
GMDVWGQGTLVTVSSAGGGG
2x_hzB04v1-gs6
(SEQ ID NO: 101)
EVQLLESGGGLVQPGGSLRLSCAASGRAFSNYAMSWFRQAPGKGLEFVSAINWNGENRYYADSV
KGRFTISRDNSKNTLYLQMNSLRAEDTAVYYCAAALSFRIGGEPYGDAYWGQGTLVTVSGSGGG
GSEVQLLESGGGLVQPGGSLRLSCAASGRAFSNYAMSWFRQAPGKGLEFVSAINWNGENRYYAD
SVKGRFTISRDNSKNTLYLQMNSLRAEDTAVYYCAAALSFRIGGEPYGDAYWGQGTLVTVSSAG
GGG
2x_hzB04v1-gs12
(SEQ ID NO: 102)
EVQLLESGGGLVQPGGSLRLSCAASGRAFSNYAMSWFRQAPGKGLEFVSAINWNGENRYYADSV
KGRFTISRDNSKNTLYLQMNSLRAEDTAVYYCAAALSFRLGGEPYGDAYWGQGTLVTVSSGGGS
GGSGGGGSEVQLLESGGGLVQPGGSLRLSCAASGRAFSNYAMSWFRQAPGKGLEFVSAINWNGE
NRYYADSVKGRFTISRDNSKNTLYLQMNSLRAEDTAVYYCAAALSFRIGGEPYGDAYWGQGTLV
TVSSAGGGG
2x_hzB04v1-gs15
(SEQ ID NO: 103)
EVQLLESGGGLVQPGGSLRLSCAASGRAFSNYAMSWFRQAPGKGLEFVSAINWNGENRYYADSV
KGRFTISRDNSKNTLYLQMNSLRAEDTAVYYCAAALSFRLGGEPYGDAYWGQGTLVTVSSGGGG
SGGGGSGGGGSEVQLLESGGGLVQPGGSLRLSCAASGRAFSNYAMSWFRQAPGKGLEFVSAINW
NGENRYYADSVKGRFTISRDNSKNTLYLQMNSLRAEDTAVYYCAAALSFRLGGEPYGDAYWGQG
TLVTVSSAGGGG
2x_F03v2-gs6
(SEQ ID NO: 104)
EVQLLESGGGLVQPGGSLRLSCAASGRSISNYAMGWFRQAPGKEREFVSASVWNNGGNYYADSV
KGRFTISRDNSKNTLYLQMNSLRAEDTAVYYCVVARTPETPITSARGANYWGQGTLVTVSGSGG
GGSEVQLLESGGGLVQPGGSLRLSCAASGRSISNYAMGWFRQAPGKEREFVSASVWNNGGNYYA
DSVKGRFTISRDNSKNTLYLQMNSLRAEDTAVYYCVVARTPETPITSARGANYWGQGTLVTVSS
AGGGG
2x_F03v1-gs6
(SEQ ID NO: 105)
EVQLLESGGGKVQPGGSLRLSCAASGRSISNYAMSWFRQAPGKGLEFVSASVWNNGGNYYADSV
KGRFTISRDNSKNTLYLQMNSLRAEDTAVYYCVVARTPETPITSARGANYWGQGTLVTVSGSGG
GGSEVQLLESGGGLVQPGGSLRLSCAASGRSISNYAMSWFRQAPGKGLEFVSASVWNNGGNYYA
DSVKGRFTISRDNSKNTLYLQMNSLRAEDTAVYYCVVARTPETPITSARGANYWGQGTLVTVSS
AGGGG
2x_F03v1-gs9
(SEQ ID NO: 106)
EVQLLESGGGLVQPGGSLRLSCAASGRSISNYAMSWFRQAPGKGLEFVSASVWNNGGNYYADSV
KGRFTISRDNSKNTLYLQMNSLRAEDTAVYYCVVARTPETPITSARGANYWGQGTLVTVSSGGG
SGGGGSEVQLLESGGGLVQPGGSLRLSCAASGRSISNYAMSWFRQAPGKGLEFVSASVWNNGGN
YYADSVKGRFTISRDNSKNTLYLQMNSLRAEDTAVYYCVVARTPETPITSARGANYWGQGTLVT
VSSAGGGG
2x_F03v1-gs12
(SEQ ID NO: 107)
EVQLLESGGGLVQPGGSLRLSCAASGRSISNYAMSWFRQAPGKGLEFVSASVWNNGGNYYADSV
KGRFTISRDNSKNTLYLQMNSLRAEDTAVYYCVVARTPETPITSARGANYWGQGTLVTVSSGGG
SGGSGGGGSEVQLLESGGGLVQPGGSLRLSCAASGRSISNYAMSWFRQAPGKGLEFVSASVWNN
GGNYYADSVKGRFTISRDNSKNTLYLQMNSLRAEDTAVYYCVVARTPETPITSARGANYWGQGT
LVTVSSAGGGG
2x_F03v1-gs15
(SEQ ID NO: 108)
EVQLLESGGGKVQPGGSLRLSCAASGRSISNYAMSWFRQAPGKGLEFVSASVWNNGGNYYADSV
KGRFTISRDNSKNTLYLQMNSLRAEDTAVYYCVVARTPETPITSARGANYWGQGTLVTVSGSGG
GGSEVQLLESGGGLVQPGGSLRLSCAASGRSISNYAMSWFRQAPGKGLEFVSASVWNNGGNYYA
DSVKGRFTISRDNSKNTLYLQMNSLRAEDTAVYYCVVARTPETPITSARGANYWGQGTLVTVSS
AGGGG
2x_hzH10v2-gs6
(SEQ ID NO: 109)
EVQLLESGGGLVQPGGSLRLSCAASVSTFGTSPVGWFRQAPGKEREFVSAIRWDGVGAYYADSV
KGRFTISRDNSKNTLYLQMNSLRAEDTAVYYCALPRRGDSELPSTVKEYGYWGQGTLVTVSGSG
GGGSEVQLLESGGGLVQPGGSLRLSCAASVSTFGTSPVGWFRQAPGKEREFVSAIRWDGVGAYY
ADSVKGRFTISRDNSKNTLYLQMNSLRAEDTAVYYCALPRRGDSELPSTVKEYGYWGQGTLVTV
SSAGGGG
2x_hzH10v2-gs15
(SEQ ID NO: 110)
EVQLLESGGGLVQPGGSLRLSCAASVSTFGTSPVGWFRQAPGKEREFVSAIRWDGVGAYYADSV
KGRFTISRDNSKNTLYLQMNSLRAEDTAVYYCALPRRGDSELPSTVKEYGYWGQGTLVTVSSGG
GGSGGGGSGGGGSEVQLLESGGGLVQPGGSLRLSCAASVSTFGTSPVGWFRQAPGKEREFVSAI
RWDGVGAYYADSVKGRFTISRDNSKNTLYLQMNSLRAEDTAVYYCALPRRGDSELPSTVKEYGY
WGQGTLVTVSSAGGGG
2x_hz1F5v3_gs6
(SEQ ID NO: 111)
EVQLLESGGGEVQPGGSLRLSCAASGLTFPNYGMGWFRQAPGKEREFVSAIYWSGGTVFYAESV
KGRFTISRDNAKNTVYLQMSSLRAEDTAVYYCAVTIRGAATQTWKYDYWGQGTLVTVKPGGSGG
SEVQLLESGGGEVQPGGSLRLSCAASGLTFPNYGMGWFRQAPGKEREFVSAIYWSGGTVFYAES
VKGRFTISRDNAKNTVYLQMSSLRAEDTAVYYCAVTIRGAATQTWKYDYWGQGTLVTVKPGGGG
DKTHTCPPC
2x_hz1F5v4_gs6
(SEQ ID NO: 112)
EVQLLESGGGEVQPGGSLRLSCAASGLTFPNYGMGWFRQAPGKEREFLAVIYWSGGTVFYAESV
KGRFTISRDNAKNTVYLQMSSLRAEDTAVYYCAVTIRGAATQTWKYDYWGQGTLVTVKPGGSGG
SEVQLLESGGGEVQPGGSLRLSCAASGLTFPNYGMGWFRQAPGKEREFLAVIYWSGGTVEYAES
VKGRFTISRDNAKNTVYLQMSSLRAEDTAVYYCAVTIRGAATQTWKYDYWGQGTLVTVKPGGGG
DKTHTCPPC
2x_hz1F5v5_gs6
(SEQ ID NO: 113)
EVQLLESGGGEVQPGGSLRLSCAASGLTFPNYGMGWFRQAPGKEREFVSAIYWSGGTVYYAESV
KGRFTISRDNAKNTLYLQMSSLRAEDTAVYYCAVTIRGAATQTWKYDYWGQGTLVTVKPGGSGG
SEVQLLESGGGEVQPGGSLRLSCAASGLTFPNYGMGWFRQAPGKEREFVSAIYWSGGTVYYAES
VKGRFTISRDNAKNTLYLQMSSLRAEDTAVYYCAVTIRGAATQTWKYDYWGQGTLVTVKPGGGG
DKTHTCPPC
2x_hz1F5v6_gs6
(SEQ ID NO: 114)
EVQLLESGGGEVQPGGSLRLSCAASGLTFPNYGMGWFRQAPGKEREFLAVIYWSGGTVYYAESV
KGRFTISRDNAKNTLYLQMSSLRAEDTAVYYCAVTIRGAATQTWKYDYWGQGTLVTVKPGGSGG
SEVQLLESGGGEVQPGGSLRLSCAASGLTFPNYGMGWFRQAPGKEREFLAVIYWSGGTVYYAES
VKGRFTISRDNAKNTLYLQMSSLRAEDTAVYYCAVTIRGAATQTWKYDYWGQGTLVTVKPGGGG
DKTHTCPPC
2x_hz1F5v7_gs6
(SEQ ID NO: 115)
EVQLLESGGGEVQPGGSLRLSCAASGLTFPNYGMGWFRQAPGKEREFVSAIYWSGGTVYYAESV
KGRFTISRDNAKNTVYLQMSSLRAEDTAVYYCAVTIRGAATQTWKYDYWGQGTLVTVKPGGSGG
SEVQLLESGGGEVQPGGSLRLSCAASGLTFPNYGMGWFRQAPGKEREFVSAIYWSGGTVYYAES
VKGRFTISRDNAKNTVYLQMSSLRAEDTAVYYCAVTIRGAATQTWKYDYWGQGTLVTVKPGGGG
DKTHTCPPC
2x_hz1F5v8_gs6
(SEQ ID NO: 116)
EVQLLESGGGEVQPGGSLRLSCAASGLTFPNYGMGWFRQAPGKEREFLAVIYWSGGTVYYAESV
KGRFTISRDNAKNTVYLQMSSLRAEDTAVYYCAVTIRGAATQTWKYDYWGQGTLVTVKPGGSGG
SEVQLLESGGGEVQPGGSLRLSCAASGLTFPNYGMGWFRQAPGKEREFLAVIYWSGGTVYYAES
VKGRFTISRDNAKNTVYLQMSSLRAEDTAVYYCAVTIRGAATQTWKYDYWGQGTLVTVKPGGGG
DKTHTCPPC
2x_hzC06v2_gs6
(SEQ ID NO: 117)
EVQLLESGGGEVQPGGSLRLSCAASGRTVSNYAMGWFRQAPGKDREFVSALNWGGDTTYYAESV
KGRFTISRDNAKNTLYLQMSSLRAEDTAVYYCAAAQSFRRGGAPYGDNYWGQGTLVTVKPGGSG
GSEVQLLESGGGEVQPGGSLRLSCAASGRTVSNYAMGWFRQAPGKDREFVSALNWGGDTTYYAE
SVKGRFTISRDNAKNTLYLQMSSLRAEDTAVYYCAAAQSFRRGGAPYGDNYWGQGTLVTVKPGG
GGDKTHTCPPC
2x_hzC06v2_gs9
(SEQ ID NO: 118)
EVQLLESGGGEVQPGGSLRLSCAASGRTVSNYAMGWFRQAPGKDREFVSALNWGGDTTYYAESV
KGRFTISRDNAKNTLYLQMSSLRAEDTAVYYCAAAQSFRRGGAPYGDNYWGQGTLVTVKPGGSG
GSGGSEVQLLESGGGEVQPGGSLRLSCAASGRTVSNYAMGWFRQAPGKDREFVSALNWGGDTTY
YAESVKGRFTISRDNAKNTLYLQMSSLRAEDTAVYYCAAAQSFRRGGAPYGDNYWGQGTLVTVK
PGGGGDKTHTCPPC
3x_hzF03
(SEQ ID NO: 119)
EVQLLESGGGLVQPGGSLRLSCAASGRSISNYAMSWFRQAPGKGLEFVSASVWNNGGNYYADSV
KGRFTISRDNSKNTLYLQMNSLRAEDTAVYYCVVARTPETPITSARGANYWGQGTLVTVSSGGG
GSGGGGSGGGGSEVQLLESGGGLVQPGGSLRLSCAASGRSISNYAMSWFRQAPGKGLEFVSASV
WNNGGNYYADSVKGRFTISRDNSKNTLYLQMNSLRAEDTAVYYCVVARTPETPITSARGANYWG
QGTLVTVSSGGGGSGGGGSGGGGSEVQLLESGGGLVQPGGSLRLSCAASGRSISNYAMSWFRQA
PGKGLEFVSASVWNNGGNYYADSVKGRFTISRDNSKNTLYLQMNSLRAEDTAVYYCVVARTPET
PITSARGANYWGQGTLVTVSSAGGGG
3x_H10-DS
(SEQ ID NO: 120)
EVQLLESGGGEVQPGGSLRLSCAASVSTFGTSPVGWFRQAPGKEREFVCAIRWEGVGAYYAESV
KGRFTCSRDNAKNTLYLQMSSLRAEDTAVYYCALPRRGDSELPSTVKEYGYWGQGTLVTVKPGG
SGGSEVQLLESGGGEVQPGGSLRLSCAASVSTFGTSPVGWFRQAPGKEREFVCAIRWEGVGAYY
AESVKGRFTCSRDNAKNTLYLQMSSLRAEDTAVYYCALPRRGDSELPSTVKEYGYWGQGTLVTV
KPGGSGGSEVQLLESGGGEVQPGGSLRLSCAASVSTFGTSPVGWFRQAPGKEREFVCAIRWEGV
GAYYAESVKGRFTCSRDNAKNTLYLQMSSLRAEDTAVYYCALPRRGDSELPSTVKEYGYWGQGT
LVTVKPGGGG
3x_H10
(SEQ ID NO: 121)
EVQLLESGGGEVQPGGSLRLSCAASVSTFGTSPVGWFRQAPGKEREFVSAIRWEGVGAYYAESV
KGRFTISRDNAKNTLYLQMSSLRAEDTAVYYCALPRRGDSELPSTVKEYGYWGQGTLVTVKPGG
SGGSEVQLLESGGGEVQPGGSLRLSCAASVSTFGTSPVGWFRQAPGKEREFVSAIRWEGVGAYY
AESVKGRFTISRDNAKNTLYLQMSSLRAEDTAVYYCALPRRGDSELPSTVKEYGYWGQGTLVTV
KPGGSGGSEVQLLESGGGEVQPGGSLRLSCAASVSTFGTSPVGWFRQAPGKEREFVSAIRWEGV
GAYYAESVKGRFTISRDNAKNTLYLQMSSLRAEDTAVYYCALPRRGDSELPSTVKEYGYWGQGT
LVTVKPGGGG
3x_1F2-DS
(SEQ ID NO: 122)
EVQLLESGGGEVQPGGSLRLSCAASGSTFSSLDMGWFRQAPGKGREFVCAISRSGDNIYYAESV
KGRFTCSRDNAKNTLYLQMSSLRAEDTAVYYCAVESQPTYSGGVYYPRYGMDVWGQGTLVTVKP
GGSGGSEVQLLESGGGEVQPGGSLRLSCAASGSTFSSLDMGWFRQAPGKGREFVCAISRSGDNI
YYAESVKGRFTCSRDNAKNTLYLQMSSLRAEDTAVYYCAVESQPTYSGGVYYPRYGMDVWGQGT
LVTVKPGGSGGSEVQLLESGGGEVQPGGSLRLSCAASGSTFSSLDMGWFRQAPGKGREFVCAIS
RSGDNIYYAESVKGRFTCSRDNAKNTLYLQMSSLRAEDTAVYYCAVESQPTYSGGVYYPRYGMD
VWGQGTLVTVKPGGGG
3x_1F2
(SEQ ID NO: 123)
EVQLLESGGGEVQPGGSLRLSCAASGSTFSSLDMGWFRQAPGKGREFVSAISRSGDNIYYAESV
KGRFTISRDNAKNTLYLQMSSLRAEDTAVYYCAVESQPTYSGGVYYPRYGMDVWGQGTLVTVKP
GGSGGSEVQLLESGGGEVQPGGSLRLSCAASGSTFSSLDMGWFRQAPGKGREFVSAISRSGDNI
YYAESVKGRFTISRDNAKNTLYLQMSSLRAEDTAVYYCAVESQPTYSGGVYYPRYGMDVWGQGT
LVTVKPGGSGGSEVQLLESGGGEVQPGGSLRLSCAASGSTFSSLDMGWFRQAPGKGREFVSAIS
RSGDNIYYAESVKGRFTISRDNAKNTLYLQMSSLRAEDTAVYYCAVESQPTYSGGVYYPRYGMD
VWGQGTLVTVKPGGGG
3x_H10-gs15
(SEQ ID NO: 124)
QVQLVQSGGGLVQAGGSLTLSCAASVSTFGTSPVGWFRQAPGKEREFVSAIRWDGVGAYYADSV
RGRFKNSKDNAKRTAYLQMNRLKPEDTAVYYCALPRRGDSELPSTVKEYGYWGQGTQVTVSSGG
GGSGGGGSGGGGSQVQLVQSGGGLVQAGGSLTLSCAASVSTFGTSPVGWFRQAPGKEREFVSAI
RWDGVGAYYADSVRGRFKNSKDNAKRTAYLQMNRLKPEDTAVYYCALPRRGDSELPSTVKEYGY
WGQGTQVTVSSGGGGSGGGGSGGGGSQVQLVQSGGGLVQAGGSLTLSCAASVSTFGTSPVGWER
QAPGKEREFVSAIRWDGVGAYYADSVRGRFKNSKDNAKRTAYLQMNRLKPEDTAVYYCALPRRG
DSELPSTVKEYGYWGQGTQVTVSSAGGGG
TAS266/11H6_hu_tetramer
(SEQ ID NO: 125)
EVQLLESGGGLVQPGGSLRLSCAASGTFDKINNMGWYRQAPGKQRDLVAQITPGGITDYADSVK
GRFTISRDNSKNTLYLQMNSLRPEDTAVYYCNAEILKRAYIDVYVNYWGQGTLVTVSSGGGGSG
GGGSGGGGSGGGGSGGGGSGGGGSGGGGSEVQLLESGGGLVQPGGSLRLSCAASGTFDKINNMG
WYRQAPGKQRDLVAQITPGGITDYADSVKGRFTISRDNSKNTLYLQMNSLRPEDTAVYYCNAEI
LKRAYIDVYVNYWGQGTLVTVSSGGGGSGGGGSGGGGSGGGGSGGGGSGGGGSGGGGSEVQLLE
SGGGLVQPGGSLRLSCAASGTFDKINNMGWYRQAPGKQRDLVAQITPGGITDYADSVKGRFTIS
RDNSKNTLYLQMNSLRPEDTAVYYCNAEILKRAYIDVYVNYWGQGTLVTVSSGGGGSGGGGSGG
GGSGGGGSGGGGSGGGGSGGGGSEVQLLESGGGLVQPGGSLRLSCAASGTFDKINNMGWYRQAP
GKQRDLVAQITPGGITDYADSVKGRFTISRDNSKNTLYLQMNSLRPEDTAVYYCNAEILKRAYI
DVYVNYWGQGTLVTVSS
FIX-TAS266
(SEQ ID NO: 126)
EVQLLESGGGEVQPGGSLRLSCAASGTFDKINNMGWYRQAPGKQRDLVAQITPGGITDYADSVK
GRFTISRDNSKNTLYLQMNSLRPEDTAVYYCNAEILKRAYIDVYVNYWGQGTLVTVKPGGGGSG
GGGSGGGGSGGGGSGGGGGGGGSGGGGSEVQLLESGGGEVQPGGSLRLSCAASGTFDKINNMG
WYRQAPGKQRDLVAQITPGGITDYADSVKGRFTISRDNSKNTLYLQMNSLRPEDTAVYYCNAEI
LKRAYIDVYVNYWGQGTLVTVKPGGGGSGGGGSGGGGSGGGGSGGGGSGGGGSGGGGSEVQLLE
SGGGEVQPGGSLRLSCAASGTFDKINNMGWYRQAPGKQRDLVAQITPGGITDYADSVKGRFTIS
RDNSKNTLYLQMNSLRPEDTAVYYCNAEILKRAYIDVYVNYWGQGTLVTVKPGGGGSGGGGSGG
GGSGGGGSGGGGSGGGGSGGGGSEVQLLESGGGEVQPGGSLRLSCAASGTFDKINNMGWYRQAP
GKQRDLVAQITPGGITDYADSVKGRFTISRDNSKNTLYLQMNSLRPEDTAVYYCNAEILKRAYI
DVYVNYWGQGTLVTVKPGG

The DR5-targeting proteins described herein are useful in a variety of therapeutic, diagnostic and prophylactic indications. For example, the DR5-targeting proteins are useful in treating a variety of diseases and disorders in a subject. In some embodiments, the DR5-targeting proteins are useful in treating, alleviating a symptom of, ameliorating and/or delaying the progression of a disease or disorder in a subject suffering from or identified as being at risk for an inflammatory disease or disorder. In some embodiments, the DR5-targeting proteins are useful in treating, alleviating a symptom of, ameliorating and/or delaying the progression of a cancer or other neoplastic condition. In some embodiments, the cancer is bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, mesothelioma, leukemia, lymphoma, myeloma, and virus-related cancer. In certain embodiments, the cancer is a metastatic cancer, refractory cancer, or recurrent cancer. In some embodiments, the DR5-targeting proteins are useful in reducing or depleting the number of T regulatory cells in a tumor of a subject in need thereof. In some embodiments, the DR5-targeting proteins are useful in stimulating an immune response in a subject. In some embodiments, the DR5-targeting proteins are useful in treating, alleviating a symptom of, ameliorating and/or delaying the progression of an autoimmune disease or disorder. In some embodiments, the DR5-targeting proteins are useful in treating, alleviating a symptom of, ameliorating and/or delaying the progression of viral, bacterial and parasitic infections.

Therapeutic formulations of the disclosure, which include a DR5-targeting molecule of the disclosure, are used to treat or alleviate a symptom associated with a disease or disorder associated with aberrant activity and/or expression of DR5 in a subject. A therapeutic regimen is carried out by identifying a subject, e.g., a human patient suffering from (or at risk of developing) a disease or disorder associated with aberrant activity and/or expression of DR5 using standard methods, including any of a variety of clinical and/or laboratory procedures. The term patient includes human and veterinary subjects. The term subject includes humans and other mammals.

Efficaciousness of treatment is determined in association with any known method for diagnosing or treating the particular disease or disorder associated with aberrant activity and/or expression of DR5. Alleviation of one or more symptoms of the disease or disorder associated with aberrant activity and/or expression of DR5 indicates that the DR5-targeting molecule confers a clinical benefit.

Therapeutic uses of the DR5-targeting molecules of the disclosure can also include the administration of one or more additional agents.

In some embodiments, the DR5-targeting molecule is administered during and/or after treatment in combination with one or more additional agents. In some embodiments, the DR5-targeting molecule and the additional agent are formulated into a single therapeutic composition, and the DR5-targeting molecule and additional agent are administered simultaneously. Alternatively, the DR5-targeting molecule and additional agent are separate from each other, e.g., each is formulated into a separate therapeutic composition, and the DR5-targeting molecule and the additional agent are administered simultaneously, or the DR5-targeting molecule and the additional agent are administered at different times during a treatment regimen. For example, the DR5-targeting molecule is administered prior to the administration of the additional agent, the DR5-targeting molecule is administered subsequent to the administration of the additional agent, or the DR5-targeting molecule and the additional agent are administered in an alternating fashion. As described herein, the DR5-targeting molecule and additional agent are administered in single doses or in multiple doses.

In some embodiments, the DR5-targeting molecule and the additional agent(s) are administered simultaneously. For example, the DR5-targeting molecule and the additional agent(s) can be formulated in a single composition or administered as two or more separate compositions. In some embodiments, the DR5-targeting molecule and the additional agent(s) are administered sequentially, or the DR5-targeting molecule and the additional agent are administered at different times during a treatment regimen.

Methods for the screening of DR5 targeting molecules that possess the desired specificity include, but are not limited to, enzyme linked immunosorbent assay (ELISA), enzymatic assays, flow cytometry, and other immunologically mediated techniques known within the art.

The disclosure further provides nucleic acid sequences and particularly DNA sequences that encode the present fusion proteins. Preferably, the DNA sequence is carried by a vector suited for extrachromosomal replication such as a phage, virus, plasmid, phagemid, cosmid, YAC, or episome. In particular, a DNA vector that encodes a desired fusion protein can be used to facilitate the methods of preparing the DR5-targeting molecules described herein and to obtain significant quantities of the fusion protein. The DNA sequence can be inserted into an appropriate expression vector, i.e., a vector which contains the necessary elements for the transcription and translation of the inserted protein-coding sequence. A variety of host-vector systems may be utilized to express the protein-coding sequence. These include mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA or cosmid DNA. Depending on the host-vector system utilized, any one of a number of suitable transcription and translation elements may be used.

The disclosure also provides methods of producing a DR5-targeting molecule by culturing a cell under conditions that lead to expression of the polypeptide, wherein the cell comprises an isolated nucleic acid molecule encoding a DR5-targeting molecule described herein, and/or vectors that include these isolated nucleic acid sequences. The disclosure provides methods of producing a DR5-targeting molecule by culturing a cell under conditions that lead to expression of the DR5-targeting molecule, wherein the cell comprises an isolated nucleic acid molecule encoding a DR5-targeting molecule described herein, and/or vectors that include these isolated nucleic acid sequences.

The fusion proteins of the disclosure (also referred to herein as “active compounds”), and derivatives, fragments, analogs and homologs thereof, can be incorporated into pharmaceutical compositions suitable for administration. Such compositions typically comprise the fusion protein and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a kit, container, pack, or dispenser together with instructions for administration. These pharmaceutical compositions can be included in diagnostic kits with instructions for use.

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients. The term patient includes human and veterinary subjects.

As utilized in accordance with the present disclosure, the following terms, unless otherwise indicated, shall be understood to have the following meanings:

As used herein, the terms “targeting fusion protein” and “antibody” can be synonyms. As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” “or directed against” is meant that the antibody reacts with one or more antigenic determinants of the desired antigen and does not react with other polypeptides or binds at much lower affinity (K_{d}>10⁻⁶). Antibodies include, but are not limited to, polyclonal, monoclonal, chimeric, dAb (domain antibody), single chain, Fab, Fab′ and F(ab′)₂ fragments, Fᵥ, scFvs, an Fab expression library, and single domain antibody (sdAb) fragments, for example V_{H}H, V_{NAR}, engineered V_{H} or V_{K}.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses (also known as isotypes) as well, such as IgG₁, IgG₂, and others. Furthermore, in humans, the light chain may be a kappa chain or a lambda chain.

The term “monoclonal antibody” (mAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

The single domain antibody (sdAb) fragments portions of the fusion proteins of the present disclosure are referred to interchangeably herein as targeting polypeptides herein.

As used herein, the term “epitope” includes any protein determinant capable of specific binding to/by an immunoglobulin or fragment thereof, or a T-cell receptor. The term “epitope” includes any protein determinant capable of specific binding to/by an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≤1 μM; e.g., ≤100 nM, preferably ≤10 nM and more preferably ≤1 nM.

As used herein, the terms “immunological binding” and “immunological binding properties” and “specific binding” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (kₒₙ) and the “off rate constant” (k_{off}) can be determined by calculation of the concentrations and the actual rates of association and dissociation. (See Nature 361:186-87 (1993)). The ratio of k_{off}/kₒₙ enables the cancellation of all parameters not related to affinity, and is equal to the dissociation constant K_{d}. (See, generally, Davies et al. (1990) Annual Rev Biochem 59:439-473). An antibody of the present disclosure is said to specifically bind to an antigen, when the equilibrium binding constant (K_{d}) is ≤1 μM, preferably ≤100 nM, more preferably ≤10 nM, and most preferably δ100 pM to about 1 pM, as measured by assays such as radioligand binding assays, surface plasmon resonance (SPR), flow cytometry binding assay, or similar assays known to those skilled in the art.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present disclosure, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the disclosure.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991).

The term “polypeptide fragment” as used herein refers to a polypeptide that has an amino terminal and/or carboxy-terminal deletion, but where the remaining amino acid sequence is identical to the corresponding positions in the naturally-occurring sequence deduced, for example, from a full length cDNA sequence. Fragments typically are at least 5, 6, 8 or 10 amino acids long, preferably at least 14 amino acids long' more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has specific binding to DR5, under suitable binding conditions. Typically, polypeptide analogs comprise a conservative amino acid substitution (or addition or deletion) with respect to the naturally-occurring sequence. Analogs typically are at least 20 amino acids long, preferably at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986), Veber and Freidinger TINS p.392 (1985); and Evans et al. J. Med. Chem. 30:1229 (1987). Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH--(cis and trans), —COCH₂—, CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992)); for example, by adding internal cysteine residues capable of forming intramolecular disulfide bridges which cyclize the peptide.

The term “agent” is used herein to denote a chemical compound, a mixture of chemical compounds, a biological macromolecule, and/or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

As used herein, the terms “treat,” treating,” “treatment,” and the like refer to reducing and/or ameliorating a disorder and/or symptoms associated therewith. By “alleviate” and/or “alleviating” is meant decrease, suppress, attenuate, diminish, arrest, and/or stabilize the development or progression of a disease such as, for example, a cancer. It will be appreciated that, although not precluded, treating a disorder or condition does not require that the disorder, condition or symptoms associated therewith be completely eliminated.

In this disclosure, “comprises,” “comprising,” “containing,” “having,” and the like can have the meaning ascribed to them in U.S. Patent law and can mean “includes,” “including,” and the like; the terms “consisting essentially of” or “consists essentially” likewise have the meaning ascribed in U.S. Patent law and these terms are open-ended, allowing for the presence of more than that which is recited so long as basic or novel characteristics of that which is recited are not changed by the presence of more than that which is recited, but excludes prior art embodiments.

By “effective amount” is meant the amount required to ameliorate the symptoms of a disease relative to an untreated patient. The effective amount of active compound(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “subject” is meant a mammal, including, but not limited to, a human or non-human mammal, such as a bovine, equine, canine, rodent, ovine, primate, camelid, or feline.

The term “administering,” as used herein, refers to any mode of transferring, delivering, introducing, or transporting a therapeutic agent to a subject in need of treatment with such an agent. Such modes include, but are not limited to, oral, topical, intravenous, intraperitoneal, intramuscular, intradermal, intranasal, and subcutaneous administration.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the terms “a,” “an,” and “the” are understood to be singular or plural. Unless specifically stated or obvious from context, as used herein, the term “or” is understood to be inclusive.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The invention will be further described in the following examples, which do not limit the scope of the disclosure described in the claims.

### EXAMPLES

### Example 1: Binding Assays

Binding of DR5-targeting fusion proteins was assessed by flow cytometry, using a CHO cell line stably transfected with cDNA encoding full length DR5 or cancer cell lines that endogenously express DR5. A titration series of the fusion protein was incubated with the DR5-expressing cell lines (approx. 2.5-5×10⁴ cells/well) for 30 minutes at 4° C. in FACS Buffer (PBS 1% BSA, 0.1% NaN₃ pH 7.4) in 96 well plates. Following 3 wash steps in FACS buffer, an APC-conjugated anti-human Fcγ specific secondary antibody (Jackson ImmunoResearch) was added and incubated for 30 minutes at 4° C. Following three additional wash steps in FACS buffer bound antibody was detected via flow cytometry (IQue Intellicyte). Binding of fusion proteins to cynomologus monkey DR5 (cynoDR5) was determined by ELISA wherein a recombinant protein corresponding to the extracellular domain (ECD) of cynoDR5 fused to a murine Fc region (mFc) was immobilized on Medisorp 96 well plates (Nunc). Following sufficient blocking and washing steps, bound fusion proteins were detected using an HRP-conjugated anti-human Fcγ specific secondary antibody (Jackson ImmunoResearch) and TMB reagent and absorbance read at A₆₅₀ₙₘ.

### Example 2: Apoptosis Assays

Antibody-mediated direct killing of cells was determined by measuring the amount of ATP present following a 16-48 h treatment period using CellTiter-Glo® (Promega G7572). Cancer cells were seeded at 1.5-3×10⁴ cells/well at 7×10⁴ cells/well in 96-well flat-bottom tissue culture treated plates. An alternative method for measuring cell death is to fluorescently stain cells using IncuCyte™ Caspase-3/7 Reagent for Apoptosis (Essen BioScience 4440) during antibody treatment and quantify fluorescent cells using an IncuCyte® ZOOM System some embodiments, the fusion protein contains a polypeptide. Cell lines used include Colo-205 (ATCC® CCL-222™), Panc-1 (ATCC® CRL-1469™), HCT-116 (ATCC® CCL-247™), JL-1 (DSMZ ACC 596), NCI-H28 (ATCC® CRL-5820™), NCI-H460 (ATCC® HTB-177™), HT-29 (ATCC® HTB-38™). MSTO-211H (ATCC® CRL-2081™). In some experiments, an anti-human IgG Fcγ-specific secondary (Jackson ImmunoResearch) antibody was used to crosslink and further cluster the DR5 targeting fusion proteins of the present disclosure. In other experiments 6 μM doxycycline was used to sensitize cells to DR5-mediated apoptosis.

### Example 3: Pre-Existing Autoantibodies Recognizing sdAbs

Pre-existing human anti-VH (HAVH) in human plasma or IVIG (purified IgG from pooled human plasma, trade name Gamunex®-C) were measured by ELISA. Test articles (TAS266, fusion proteins or therapeutic antibodies) were coated on an ELISA plate in PBS, the plate was blocked by 3% BSA in PBS, then human plasma or IVIG (as a source of naturally occurring HAVH) was diluted in PBS+0.1% polysorbate-20 (PBST) and allowed to bind to the plate. After washing the plate with PBST, bound plasma antibodies (HAVH) were detected by anti-light chain secondary antibodies (anti-human IgKappa or anti-IgLambda) conjugated to HRP, and developed with TMB substrate. This strategy of detecting HAVH by anti-light chain secondary antibody is compatible with test articles lacking light chains, which includes TAS266 as well as the described multivalent sdAbs, and facilitates detection of HAVH of any isotype. Control therapeutic antibodies with kappa or lambda light chains were coated and used as 100% binding reference data points to normalize the data to, and served as control IgG for the opposite secondary antibody.

### Example 4: Hepatotoxicity Assays

Primary human hepatocytes or HepRG™ (Thermo Fisher Scientific) the terminally differentiated hepatic cells derived from a hepatic progenitor cell line were used to assess DR5 agonist mediated apoptosis of hepatocytes. All assays were conducted in a similar manner to the apoptosis assays using cancer cell lines (Example 2). Pooled human IgG from multiple donors, IVIG (Gamunex®-C, Grifols), was used as source of natural sdAb-directed autoantibodies, also termed human anti-VH (HAVH) autoantibodies. In some experiments, IVIG was titrated or used at a fixed concentration. In some assays, FIX-TAS266, which is a modified version of TAS266 that is engineered to avoid recognition by HAVH autoantibodies, was included. FIX-2TAS66 includes modifications a Leu11 and the C-terminal region of each of the four DR5 sdAbs of TAS266.

1. A method of reducing or depleting the number of T regulatory cells in a tumor in a subject, comprising administering to a subject in need thereof a therapeutically effective amount of an isolated polypeptide that binds death receptor 5 (DR5) and comprises a plurality of DR5 binding domains (DR5BDs), wherein each DR5BD is a VHH comprising a CDR1 comprising the amino acid sequence of SEQ ID NO: 128, a CDR2 comprising the amino acid sequence of SEQ ID NO: 131, and a CDR3 comprising the amino acid sequence of SEQ ID NO: 130, and wherein adjacent DR5BDs are operably linked by an amino acid linker.

2.-7. (canceled)

8. The method of claim 1, wherein the plurality of DR5BDs is two DR5BDs.

9. The method of claim 1, wherein the plurality of DR5BDs is four DR5BDs.

10. The method of claim 1, wherein the plurality of DR5BDs is six DR5BDs.

11. The method of claim 1, wherein each DR5BD comprises the amino acid sequence of SEQ ID NO: 87.

12. The method of claim 8, wherein each DR5BD comprises the amino acid sequence of SEQ ID NO: 87.

13. The method of claim 9, wherein each DR5BD comprises the amino acid sequence of SEQ ID NO: 87.

14. The method of claim 10, wherein each DR5BD comprises the amino acid sequence of SEQ ID NO: 87.

15.-18. (canceled)

19. The method of claim 1, wherein the isolated polypeptide comprises an immunoglobulin hinge region and an immunoglobulin Fc region.

20. The method of claim 19, wherein the immunoglobulin Fc region is an IgG1 Fc region, an IgG2 Fc region, an IgG3 Fc region, or an IgG4 Fc region.

21. The method of claim 19, wherein the immunoglobulin Fc region comprises an amino acid sequence selected from SEQ ID NOs: 1-5 or 127.

22.-25. (canceled)

26. The method of claim 1, wherein each VHH is a humanized VHH.

27.-32. (canceled)

33. The method of claim 9, wherein the polypeptide is a homodimer of the structure: DR5BD-Linker-DR5BD-Linker-Hinge-Fc, where each the DR5BD is a humanized VHH sequence.

34.-46. (canceled)

47. The method of claim 33, wherein each DR5BD comprises the amino acid sequence of SEQ ID NO: 87.

48. The method of claim 1, wherein the polypeptide comprises the amino acid sequence of SEQ ID NO: 113.

49. The method of claim 48, wherein the polypeptide is a homodimer of the amino acid sequence of SEQ ID NO: 113 fused to an Fc region.

50. A method of reducing or depleting the number of T regulatory cells in a tumor in a subject, comprising administering to a subject in need thereof a therapeutically effective amount of an isolated polypeptide that binds death receptor 5 (DR5), wherein the polypeptide is a homodimer of the amino acid sequence of SEQ ID NO: 113 fused to an Fc region of SEQ ID NO: 2.

51. The method of claim 1, wherein each amino acid linker consists of 5-20 amino acids.

52. The method of claim 51, wherein each amino acid linker is composed predominantly of glycine and serine.

53. The method of claim 52, wherein each amino acid linker comprises an amino acid sequence selected from GGSGGS (SEQ ID NO: 11); GGSGGSGGS (SEQ ID NO: 12); GGSGGSGGSGGS (SEQ ID NO: 13); and GGSGGSGGSGGSGGS (SEQ ID NO: 14).

54. The method of claim 19, wherein the immunoglobulin hinge region comprises an amino acid sequence selected from EPKSSDKTHTCPPC (SEQ ID NO: 6), DKTHTCPPC (SEQ ID NO: 7), and ESKYGPPCPPC (SEQ ID NO: 8)

55. The method of claim 47, wherein each amino acid linker consists of 5-20 amino acids.

56. The method of claim 55, wherein each amino acid linker is composed predominantly of glycine and serine.

57. The method of claim 56, wherein each amino acid linker comprises an amino acid sequence selected from GGSGGS (SEQ ID NO: 11); GGSGGSGGS (SEQ ID NO: 12); GGSGGSGGSGGS (SEQ ID NO: 13); and GGSGGSGGSGGSGGS (SEQ ID NO: 14).

58. A method of treating, alleviating a symptom of, ameliorating and/or delaying the progression of a viral, bacterial, or parasitic infection, comprising administering to a subject in need thereof a therapeutically effective amount of an isolated polypeptide that binds death receptor 5 (DR5) and comprises a plurality of DR5 binding domains (DR5BDs), wherein each DR5BD is a VHH comprising a CDR1 comprising the amino acid sequence of SEQ ID NO: 128, a CDR2 comprising the amino acid sequence of SEQ ID NO: 131, and a CDR3 comprising the amino acid sequence of SEQ ID NO: 130, and wherein adjacent DR5BDs are operably linked by an amino acid linker.

59. A method of treating, alleviating a symptom of, ameliorating and/or delaying the progression of an autoimmune disease or disorder, comprising administering to a subject in need thereof a therapeutically effective amount of an isolated polypeptide that binds at least death receptor 5 (DR5) and comprises a plurality of DR5 binding domains (DR5BDs), wherein each DR5BD is a VHH comprising a CDR1 comprising the amino acid sequence of SEQ ID NO: 128, a CDR2 comprising the amino acid sequence of SEQ ID NO: 131, and a CDR3 comprising the amino acid sequence of SEQ ID NO: 130, and wherein adjacent DR5BDs are operably linked by an amino acid linker.


SEQUENCE LISTING (190 sequences)

SEQ ID NO: 1 - protein, 218 aa, synthetic construct
  REGION 1..218: note = chemically synthesized
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, synthetic construct
  REGION 1..215: note = chemically synthesized
PAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 217 aa, synthetic construct
  REGION 1..217: note = chemically synthesized
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 4 - protein, 218 aa, synthetic construct
  REGION 1..218: note = chemically synthesized
PAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 5 - protein, 218 aa, synthetic construct
  REGION 1..218: note = chemically synthesized
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 14 aa, synthetic construct
  REGION 1..14: note = chemically synthesized
EPKSSDKTHTCPPC

SEQ ID NO: 7 - protein, 9 aa, synthetic construct
  REGION 1..9: note = chemically synthesized
DKTHTCPPC

SEQ ID NO: 8 - protein, 11 aa, synthetic construct
  REGION 1..11: note = chemically synthesized
ESKYGPPCPPC

SEQ ID NO: 9 - protein, 12 aa, synthetic construct
  REGION 1..12: note = chemically synthesized
GQGTLVTVKPGG

SEQ ID NO: 10 - protein, 12 aa, synthetic construct
  REGION 1..12: note = chemically synthesized
GQGTLVTVEPGG

SEQ ID NO: 11 - protein, 6 aa, synthetic construct
  REGION 1..6: note = chemically synthesized
GGSGGS

SEQ ID NO: 12 - protein, 9 aa, synthetic construct
  REGION 1..9: note = chemically synthesized
GGSGGSGGS

SEQ ID NO: 13 - protein, 12 aa, synthetic construct
  REGION 1..12: note = chemically synthesized
GGSGGSGGSGGS

SEQ ID NO: 14 - protein, 15 aa, synthetic construct
  REGION 1..15: note = chemically synthesized
GGSGGSGGSGGSGGS

SEQ ID NO: 15 - protein, 123 aa, synthetic construct
  REGION 1..123: note = chemically synthesized
QVQLVQSGGGLVQAGDSLRLSCAASGLTFPNYGMGWFRQAPGEEREFLAVIYWSGGTVFYADSVKGRFTISRDAAKNMVYLQMNSLKSDDTAVYYCAVTIRGAATQTWKYDYWGRGTQVTVSS

SEQ ID NO: 16 - protein, 123 aa, synthetic construct
  REGION 1..123: note = chemically synthesized
EVQLLESGGGLVQPGGSLRLSCAASGLTFPNYGMSWFRQAPGKGLEFVSAIYWSGGTVYYADSVKGRFTISRDNSKNTLYLQMNSLRAEDTAVYYCAVTIRGAATQTWKYDYWGQGTLVTVSS

SEQ ID NO: 17 - protein, 125 aa, synthetic construct
  REGION 1..125: note = chemically synthesized
EVQLLESGGGEVQPGGSLRLSCAASGLTFPNYGMSWFRQAPGKGLEFVSAIYWSGGTVYYAESVKGRFTISRDNAKNTLYLQMSSLRAEDTAVYYCAVTIRGAATQTWKYDYWGQGTLVTVKPGG

SEQ ID NO: 18 - protein, 123 aa, synthetic construct
  REGION 1..123: note = chemically synthesized
EVQLLESGGGEVQPGGSLRLSCAASGLTFPNYGMSWFRQAPGKGLEFVSAIYWSGGTVYYAESVKGRFTISRDNAKNTLYLQMSSLRAEDTAVYYCAVTIRGAATQTWKYDYWGQGTLVTVKP

SEQ ID NO: 19 - protein, 123 aa, synthetic construct
  REGION 1..123: note = chemically synthesized
EVQLLESGGGEVQPGGSLRLSCAASGLTFPNYGMSWFRQAPGKEREFVSAIYWSGGTVYYAESVKGRFTISRDNAKNTLYLQMSSLRAEDTAVYYCAVTIRGAATQTWKYDYWGQGTQVTVKP

SEQ ID NO: 20 - protein, 125 aa, synthetic construct
  REGION 1..125: note = chemically synthesized
EVQLLESGGGEVQPGGSLRLSCAASGLTFPNYGMSWFRQAPGKGLEFVCAIYWSGGTVYYAESVKGRFTCSRDNAKNTLYLQMSSLRAEDTAVYYCAVTIRGAATQTWKYDYWGQGTLVTVKPGG

SEQ ID NO: 21 - protein, 124 aa, synthetic construct
  REGION 1..124: note = chemically synthesized
QVQLVQSGGGLVQAGGSLRLTCTASGRTVSNYAMGWFRQTPGKDREFVAALNWSGDTTSYADSVRGRFTISRDNTRNTVYLQMDSLKREDTAVYYCAAAQSFRRGGAPYGDNYWGQGTQVTVSS

SEQ ID NO: 22 - protein, 125 aa, synthetic construct
  REGION 1..125: note = chemically synthesized
EVQLLESGGGLVQPGGSLRLSCAASGRTVSNYAMSWFRQAPGKGLEFVSALNWGGDTTYYADSVKGRFTISRDNSKNTLYLQMNSLRAEDTAVYYCAAAQSFRRGGAPYGDNYWWGQGTLVTVSS

SEQ ID NO: 23 - protein, 126 aa, synthetic construct
  REGION 1..126: note = chemically synthesized
EVQLLESGGGEVQPGGSLRLSCAASGRTVSNYAMSWFRQAPGKGLEFVSALNWGGDTTYYAESVKGRFTISRDNAKNTLYLQMSSLRAEDTAVYYCAAAQSFRRGGAPYGDNYWGQGTLVTVKPGG

SEQ ID NO: 24 - protein, 124 aa, synthetic construct
  REGION 1..124: note = chemically synthesized
EVQLVQSGGGLVQAGDSLRLSCAASGRALTGYHMAWFRQAPGKEREFVTYGIWDRAGAAYADSVKGRFTMSRDNAKNTVYLQMNNLKTEDTAVYYCAASMAVRTYYSPRSYDSWGQGTQVTVSS

SEQ ID NO: 25 - protein, 124 aa, synthetic construct
  REGION 1..124: note = chemically synthesized
EVQLLESGGGLVQPGGSLRLSCAASGRALTGYHMSWFRQAPGKGREFVSYGIWDRAGAAYADSVKGRFTISRDNSKNTLYLQMNSLRAEDTAVYYCAASMAVRTYYSPRSYDSWGQGTLVTVSS

SEQ ID NO: 26 - protein, 124 aa, synthetic construct
  REGION 1..124: note = chemically synthesized
EVQLLESGGGLVQPGGSLRLSCAASGRALTGYHMSWFRQAPGKGLEFVSYGIWDRAGAAYADSVKGRFTISRDNSKNTLYLQMNSLRAEDTAVYYCAASMAVRTYYSPRSYDSWGQGTLVTVSS

SEQ ID NO: 27 - protein, 128 aa, synthetic construct
  REGION 1..128: note = chemically synthesized
EVQLVQSGGGLVQAGGSLRLSCAASGSTFSSLDMGWFRQAPGKERAFVAAISRSGDNIYYAESVKGRFTISRDNAENTTYLQMNSLKPEDSAVYYCAVDSQPTYSGGVYYPRYGMDVWGQGTQVTVSS

SEQ ID NO: 28 - protein, 10 aa, synthetic construct
  REGION 1..10: note = chemically synthesized
AIRWNEGDTY

SEQ ID NO: 29 - protein, 128 aa, synthetic construct
  REGION 1..128: note = chemically synthesized
EVQLLESGGGLVQPGGSLRLSCAASGSTFSSLDMGWFRQAPGKGREFVSAISRSGDNIYYADSVKGRFTISRDNSKNTLYLQMNSLRAEDTAVYYCAVDSQPTYSGGVYYPRYGMDVWGQGTLVTVSS

SEQ ID NO: 30 - protein, 128 aa, synthetic construct
  REGION 1..128: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDSQPTYSGGVYYPRYGMDVWGQGTLVTVKP

SEQ ID NO: 31 - protein, 12 aa, synthetic construct
  REGION 1..12: note = chemically synthesized
SGSTFSSLDMSW

SEQ ID NO: 32 - protein, 128 aa, synthetic construct
  REGION 1..128: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDTQPTYSGGVYYPRYGMDVWGQGTLVTVKP

SEQ ID NO: 33 - protein, 128 aa, synthetic construct
  REGION 1..128: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDAQPTYSGGVYYPRYGMDVWGQGTLVTVKP

SEQ ID NO: 34 - protein, 128 aa, synthetic construct
  REGION 1..128: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVESQPTYSGGVYYPRYGMDVWGQGTLVTVKP

SEQ ID NO: 35 - protein, 130 aa, synthetic construct
  REGION 1..130: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDSQPTYSGGVYYPRYGYDVWGQGTLVTVKPGG

SEQ ID NO: 36 - protein, 130 aa, synthetic construct
  REGION 1..130: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDSQPTYSGGVYYPRYGDDVWGQGTLVTVKPGG

SEQ ID NO: 37 - protein, 130 aa, synthetic construct
  REGION 1..130: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDSQPTYSGGVYYPRYGLDVWGQGTLVTVKPGG

SEQ ID NO: 38 - protein, 130 aa, synthetic construct
  REGION 1..130: note = chemically synthesized
EVQLLESGGGEVQPGGSLRLSCAASGSTFSSLDMGWFRQAPGKGREFVCAISRSGDNIYYAESVKGRFTCSRDNAKNTLYLQMSSLRAEDTAVYYCAVESQPTYSGGVYYPRYGMDVWGQGTLVTVKPGG

SEQ ID NO: 39 - protein, 130 aa, synthetic construct
  REGION 1..130: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDAQPTYSGGVYYPRYGADVWGQGTLVTVKPGG

SEQ ID NO: 40 - protein, 130 aa, synthetic construct
  REGION 1..130: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDAQPTYSGGVYYPRYGEDVWGQGTLVTVKPGG

SEQ ID NO: 41 - protein, 130 aa, synthetic construct
  REGION 1..130: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDAQPTYSGGVYYPRYGHDVWGQGTLVTVKPGG

SEQ ID NO: 42 - protein, 130 aa, synthetic construct
  REGION 1..130: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDAQPTYSGGVYYPRYGNDVWGQGTLVTVKPGG

SEQ ID NO: 43 - protein, 130 aa, synthetic construct
  REGION 1..130: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDAQPTYSGGVYYPRYGPDVWGQGTLVTVKPGG

SEQ ID NO: 44 - protein, 130 aa, synthetic construct
  REGION 1..130: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDAQPTYSGGVYYPRYGQDVWGQGTLVTVKPGG

SEQ ID NO: 45 - protein, 130 aa, synthetic construct
  REGION 1..130: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDAQPTYSGGVYYPRYGRDVWGQGTLVTVKPGG

SEQ ID NO: 46 - protein, 130 aa, synthetic construct
  REGION 1..130: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDAQPTYSGGVYYPRYGSDVWGQGTLVTVKPGG

SEQ ID NO: 47 - protein, 130 aa, synthetic construct
  REGION 1..130: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDAQPTYSGGVYYPRYGTDVWGQGTLVTVKPGG

SEQ ID NO: 48 - protein, 130 aa, synthetic construct
  REGION 1..130: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVDAQPTYSGGVYYPRYGVDVWGQGTLVTVKPGG

SEQ ID NO: 49 - protein, 124 aa, synthetic construct
  REGION 1..124: note = chemically synthesized
EVQLVQSGGGLVQAGGSLRLSCAASGRAFSNYALGWFRQAPGKEREFIAAINWNGENRYGVDSVKGRFTISRDNAQNMGYLQMNNLKPEDTAVYRCAAALSFRLGGEPYGDAYWGQGTQVTVSS

SEQ ID NO: 50 - protein, 124 aa, synthetic construct
  REGION 1..124: note = chemically synthesized
EVQLLESGGGLVQPGGSLRLSCAASGRAFSNYAMSWFRQAPGKGLEFVSAINWNGENRYYADSVKGRFTISRDNSKNTLYLQMNSLRAEDTAVYYCAAALSFRLGGEPYGDAYWGQGTLVTVSS

SEQ ID NO: 51 - protein, 122 aa, synthetic construct
  REGION 1..122: note = chemically synthesized
QVQLQESGGGLVQAGGSLRLSCVASGSIFTNNAMGWYRQAPGKQRDLVAQITMGGGITNYAPSMEGRFAISRDNAKSTVYLQMNNLKPEDTAVYYCNAEVKSADWGAYANYWGQGTQVTVSS

SEQ ID NO: 52 - protein, 122 aa, synthetic construct
  REGION 1..122: note = chemically synthesized
EVQLLESGGGLVQPGGSLRLSCAASGSIFTNNAMSWYRQAPGKGLELVSAITMGGGITYYADSVKGRFTISRDNSKNTLYLQMNSLRAEDTAVYYCNAEVKSADWGAYANYWGQGTLVTVSS

SEQ ID NO: 53 - protein, 122 aa, synthetic construct
  REGION 1..122: note = chemically synthesized
EVQLLESGGGLVQPGGSLRLSCAASGSIFTNNAMSWYRQAPGKGRELVSQITMGGGITYYADSVKGRFTISRDNSKNTLYLQMNSLRAEDTAVYYCNAEVKSADWGAYANYWGQGTLVTVSS

SEQ ID NO: 54 - protein, 125 aa, synthetic construct
  REGION 1..125: note = chemically synthesized
QVQLQESGGGLVQAGGSLRLSCAASGRSISNYAMGWFRQAPGKEREFLAASVWNNGGNYYADSVKGRFTASRDDAKSTAYLQMSRLRPEDTGIYYCVVARTPETPITSARGANYWGQGTQVTVSS

SEQ ID NO: 55 - protein, 125 aa, synthetic construct
  REGION 1..125: note = chemically synthesized
EVQLLESGGGLVQPGGSLRLSCAASGRSISNYAMGWFRQAPGKEREFVSASVWNNGGNYYADSVKGRFTISRDNSKNTLYLQMNSLRAEDTAVYYCVVARTPETPITSARGANYWGQGTLVTVSS

SEQ ID NO: 56 - protein, 127 aa, synthetic construct
  REGION 1..127: note = chemically synthesized
EVQLLESGGGEVQPGGSLRLSCAASGRSISNYAMGWFRQAPGKEREFVSASVWNNGGNYYAESVKGRFTISRDNAKNTLYLQMSSLRAEDTAVYYCVVARTPETPITSARGANYWGQGTLVTVKPGG

SEQ ID NO: 57 - protein, 127 aa, synthetic construct
  REGION 1..127: note = chemically synthesized
EVQLLESGGGEVQPGGSLRLSCAASGRSISNYAMGWFRQAPGKEREFVSASVWNNGGNYYAESVKGRFTISRDDAKSTLYLQMSSLRAEDTAVYYCVVARTPETPITSARGANYWGQGTLVTVKPGG

SEQ ID NO: 58 - protein, 127 aa, synthetic construct
  REGION 1..127: note = chemically synthesized
EVQLLESGGGEVQPGGSLRLSCAASGRSISNYAMGWFRQAPGKEREFVSASVWNQGGNYYAESVKGRFTISRDNAKNTLYLQMSSLRAEDTAVYYCVVARTPETPITSARGANYWGQGTLVTVKPGG

SEQ ID NO: 59 - protein, 127 aa, synthetic construct
  REGION 1..127: note = chemically synthesized
EVQLLESGGGEVQPGGSLRLSCAASGRSISNYAMGWFRQAPGKEREFVSASVWNNAGNYYAESVKGRFTISRDNAKNTLYLQMSSLRAEDTAVYYCVVARTPETPITSARGANYWGQGTLVTVKPGG

SEQ ID NO: 60 - protein, 127 aa, synthetic construct
  REGION 1..127: note = chemically synthesized
EVQLLESGGGEVQPGGSLRLSCAASGRSISNYAMGWFRQAPGKEREFVSASVWNQGGNYYAESVKGRFTISRDDAKSTLYLQMSSLRAEDTAVYYCVVARTPETPITSARGANYWGQGTLVTVKPGG

SEQ ID NO: 61 - protein, 127 aa, synthetic construct
  REGION 1..127: note = chemically synthesized
EVQLLESGGGEVQPGGSLRLSCAASGRSISNYAMGWFRQAPGKEREFVSASVWNNAGNYYAESVKGRFTISRDDAKSTLYLQMSSLRAEDTAVYYCVVARTPETPITSARGANYWGQGTLVTVKPGG

SEQ ID NO: 62 - protein, 127 aa, synthetic construct
  REGION 1..127: note = chemically synthesized
QVQLQESGGGSVQAGGSLTLSCAASGRAASDYAVGWFRQAPGKEREFVAACNWSGEDTVYAYIVKGRFTISRDNAGNTVSLRMSSLEPEDTAVYYCAAAPSFSRSVLDGNLSQIDYWGQGTQVTVSS

SEQ ID NO: 63 - protein, 127 aa, synthetic construct
  REGION 1..127: note = chemically synthesized
EVQLLESGGGLVQPGGSLRLSCAASGRAASDYAMSWFRQAPGKGLEFVSAINWGGEDTVYADSVKGRFTISRDNSKNTLYLQMNSLRAEDTAVYYCAAAPSFSRSVLDGNLSQIDYWGQGTLVTVSS

SEQ ID NO: 64 - protein, 124 aa, synthetic construct
  REGION 1..124: note = chemically synthesized
QVQLVQSGGGLAQAGGSLRLSCVASGRTFTNYAMGWFRQAPGKEREFVAAINWSGDSTYHADSVKGRFTISRDNAKDSVYLQMTKLKPEDTADYYCASAESFSRGGLPYGMNYWGQGTQVTVSS

SEQ ID NO: 65 - protein, 124 aa, synthetic construct
  REGION 1..124: note = chemically synthesized
EVQLLESGGGLVQPGGSLRLSCAASGRTFTNYAMSWFRQAPGKGLEFVSAINWSGDSTYYADSVKGRFTISRDNSKNTLYLQMNSLRAEDTAVYYCASAESFSRGGLPYGMNYWGQGTLVTVSS

SEQ ID NO: 66 - protein, 126 aa, synthetic construct
  REGION 1..126: note = chemically synthesized
EVQLLESGGGEVQPGGSLRLSCAASGRTFTNYAMSWFRQAPGKGLEFVSAINWSGDSTYYAESVKGRFTISRDNAKNTLYLQMSSLRAEDTAVYYCASAESFSRGGLPYGMNYWGQGTLVTVKPGG

SEQ ID NO: 67 - protein, 126 aa, synthetic construct
  REGION 1..126: note = chemically synthesized
QVQLVQSGGGLVQAGGSLTLSCAASVSTFGTSPVGWFRQAPGKEREFVSAIRWDGVGAYYADSVRGRFKNSKDNAKRTAYLQMNRLKPEDTAVYYCALPRRGDSELPSTVKEYGYWGQGTQVTVSS

SEQ ID NO: 68 - protein, 126 aa, synthetic construct
  REGION 1..126: note = chemically synthesized
EVQLLESGGGEVQPGGSLRLSCAASVSTFGTSPVGWFRQAPGKEREFVSAIRWEGVGAYYAESVKGRFTISRDNAKNTLYLQMSSLRAEDTAVYYCALPRRGDSELPSTVKEYGYWGQGTLVTVKP

SEQ ID NO: 69 - protein, 126 aa, synthetic construct
  REGION 1..126: note = chemically synthesized
EVQLLESGGGLVQPGGSLRLSCAASVSTFGTSPVGWFRQAPGKEREFVSAIRWDGVGAYYADSVKGRFTISRDNSKNTLYLQMNSLRAEDTAVYYCALPRRGDSELPSTVKEYGYWGQGTLVTVSS

SEQ ID NO: 70 - protein, 128 aa, synthetic construct
  REGION 1..128: note = chemically synthesized
EVQLLESGGGEVQPGGSLRLSCAASVSTFGTSPVGWFRQAPGKEREFVSAIRWDGVGAYYAESVKGRFTISRDNAKNTLYLQMSSLRAEDTAVYYCALPRRGDSELPSTVKEYGYWGQGTLVTVKPGG

SEQ ID NO: 71 - protein, 128 aa, synthetic construct
  REGION 1..128: note = chemically synthesized
EVQLLESGGGEVQPGGSLRLSCAASVSTFGTSPVGWFRQAPGKEREFVCAIRWEGVGAYYAESVKGRFTCSRDNAKNTLYLQMSSLRAEDTAVYYCALPRRGDSELPSTVKEYGYWGQGTLVTVKPGG

SEQ ID NO: 72 - protein, 128 aa, synthetic construct
  REGION 1..128: note = chemically synthesized
EVQLLESGGGEVQPGGSLRLSCAASVSTFGTSPVGWFRQAPGKEREFVSAIRWDAVGAYYAESVKGRFTISKDNAKRTLYLQMSSLRAEDTAVYYCALPRRGDSELPSTVKEYGYWGQGTLVTVKPGG

SEQ ID NO: 73 - protein, 128 aa, synthetic construct
  REGION 1..128: note = chemically synthesized
EVQLLESGGGEVQPGGSLRLSCAASVSTFGTSPVGWFRQAPGKEREFVSAIRWDGVGAYYAESVKGRFTISKDNAKRTLYLQMSSLRAEDTAVYYCALPRRGESELPSTVKEYGYWGQGTLVTVKPGG

SEQ ID NO: 74 - protein, 128 aa, synthetic construct
  REGION 1..128: note = chemically synthesized
EVQLLESGGGEVQPGGSLRLSCAASVSTFGTSPVGWFRQAPGKEREFVSAIRWDGVGAYYAESVKGRFTISKDNAKRTLYLQMSSLRAEDTAVYYCALPRRGDAELPSTVKEYGYWGQGTLVTVKPGG

SEQ ID NO: 75 - protein, 128 aa, synthetic construct; identical to SEQ ID NO: 70
  REGION 1..128: note = chemically synthesized

SEQ ID NO: 76 - protein, 128 aa, synthetic construct
  REGION 1..128: note = chemically synthesized
EVQLLESGGGEVQPGGSLRLSCAASVSTFGTSPVGWFRQAPGKEREFVSAIRWEGVGAYYAESVKGRFTISKDNAKRTLYLQMSSLRAEDTAVYYCALPRRGESELPSTVKEYGYWGQGTLVTVKPGG

SEQ ID NO: 77 - protein, 128 aa, synthetic construct
  REGION 1..128: note = chemically synthesized
EVQLLESGGGEVQPGGSLRLSCAASVSTFGTSPVGWFRQAPGKEREFVSAIRWEGVGAYYAESVKGRFTISKDNAKRTLYLQMSSLRAEDTAVYYCALPRRGDAELPSTVKEYGYWGQGTLVTVKPGG

SEQ ID NO: 78 - protein, 128 aa, synthetic construct
  REGION 1..128: note = chemically synthesized
EVQLLESGGGEVQPGGSLRLSCAASVSTFGTSPVGWFRQAPGKEREFVSAIRWDAVGAYYAESVKGRFTISKDNAKRTLYLQMSSLRAEDTAVYYCALPRRGESELPSTVKEYGYWGQGTLVTVKPGG

SEQ ID NO: 79 - protein, 121 aa, synthetic construct
  REGION 1..121: note = chemically synthesized
QLQLQESGGGLVQAGDSLRLSCQVSGRTLSAYLMAWFRQAPNKVREYLGRIRWNEGDTYYPDSVKGRFTISKDDAKNTVYLRMNSLKPEDTAVYYCAARSIFNPSDQYVYWGQGTQVTVSS

SEQ ID NO: 80 - protein, 121 aa, synthetic construct
  REGION 1..121: note = chemically synthesized
EVQLLESGGGLVQPGGSLRLSCAASGRTLSAYLMSWFRQAPGKGLEYVSAIRWNEGDTYYADSVKGRFTISRDNSKNTLYLQMNSLRAEDTAVYYCAARSIFNPSDQYVYWGQGTLVTVSS

SEQ ID NO: 81 - protein, 121 aa, synthetic construct
  REGION 1..121: note = chemically synthesized
EVQLLESGGGLVQPGGSLRLSCAVSGRTLSAYLMSWFRQAPGKGREYVSRIRWNEGDTYYADSVKGRFTISRDNSKNTLYLQMNSLKAEDTAVYYCAARSIFNPSDQYVYWGQGTLVTVSS

SEQ ID NO: 82 - protein, 128 aa, synthetic construct
  REGION 1..128: note = chemically synthesized
EVQLVQSGGGLVQAGGSLRLSCAASGSTFSSLDMGWFRQAPGKERAFVAAISRSGDNIYYAESVKGRFTISRDNAENTMYLQMNSLKPEDSAVYYCAVESQPTYSGGVYYPRYGMDVWGQGTQVTVSS

SEQ ID NO: 83 - protein, 128 aa, synthetic construct
  REGION 1..128: note = chemically synthesized
EVQLLESGGGLVQPGGSLRLSCAASGSTFSSLDMSWFRQAPGKGLEFVSAISRSGDNIYYADSVKGRFTISRDNSKNTLYLQMNSLRAEDTAVYYCAVESQPTYSGGVYYPRYGMDVWGQGTLVTVSS

SEQ ID NO: 84 - protein, 128 aa, synthetic construct
  REGION 1..128: note = chemically synthesized
EVQLLESGGGLVQPGGSLRLSCAASGSTFSSLDMGWFRQAPGKGREFVSAISRSGDNIYYADSVKGRFTISRDNSKNTLYLQMNSLRAEDTAVYYCAVESQPTYSGGVYYPRYGMDVWGQGTLVTVSS

SEQ ID NO: 85 - protein, 123 aa, synthetic construct
  REGION 1..123: note = chemically synthesized
EVQLLESGGGEVQPGGSLRLSCAASGLTFPNYGMGWFRQAPGKEREFVSAIYWSGGTVFYAESVKGRFTISRDNAKNTVYLQMSSLRAEDTAVYYCAVTIRGAATQTWKYDYWGQGTLVTVKP

SEQ ID NO: 86 - protein, 123 aa, synthetic construct
  REGION 1..123: note = chemically synthesized
EVQLLESGGGEVQPGGSLRLSCAASGLTFPNYGMGWFRQAPGKEREFLAVIYWSGGTVFYAESVKGRFTISRDNAKNTVYLQMSSLRAEDTAVYYCAVTIRGAATQTWKYDYWGQGTLVTVKP

SEQ ID NO: 87 - protein, 123 aa, synthetic construct
  REGION 1..123: note = chemically synthesized
EVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKP

SEQ ID NO: 88 - protein, 123 aa, synthetic construct
  REGION 1..123: note = chemically synthesized
EVQLLESGGGEVQPGGSLRLSCAASGLTFPNYGMGWFRQAPGKEREFLAVIYWSGGTVYYAESVKGRFTISRDNAKNTLYLQMSSLRAEDTAVYYCAVTIRGAATQTWKYDYWGQGTLVTVKP

SEQ ID NO: 89 - protein, 123 aa, synthetic construct
  REGION 1..123: note = chemically synthesized
EVQLLESGGGEVQPGGSLRLSCAASGLTFPNYGMGWFRQAPGKEREFVSAIYWSGGTVYYAESVKGRFTISRDNAKNTVYLQMSSLRAEDTAVYYCAVTIRGAATQTWKYDYWGQGTLVTVKP

SEQ ID NO: 90 - protein, 123 aa, synthetic construct
  REGION 1..123: note = chemically synthesized
EVQLLESGGGEVQPGGSLRLSCAASGLTFPNYGMGWFRQAPGKEREFLAVIYWSGGTVYYAESVKGRFTISRDNAKNTVYLQMSSLRAEDTAVYYCAVTIRGAATQTWKYDYWGQGTLVTVKP

SEQ ID NO: 91 - protein, 124 aa, synthetic construct
  REGION 1..124: note = chemically synthesized
EVQLLESGGGEVQPGGSLRLSCAASGRTVSNYAMGWFRQAPGKDREFVSALNWGGDTTYYAESVKGRFTISRDNAKNTLYLQMSSLRAEDTAVYYCAAAQSFRRGGAPYGDNYWGQGTLVTVKP

SEQ ID NO: 92 - protein, 256 aa, synthetic construct
  REGION 1..256: note = chemically synthesized
EVQLLESGGGEVQPGGSLRLSCAASGLTFPNYGMSWFRQAPGKGLEFVCAIYWSGGTVYYAESVKGRFTCSRDNAKNTLYLQMSSLRAEDTAVYYCAVTIRGAATQTWKYDYWGQGTLVTVKPGGSGGSEVQLLESGGGEVQPGGSLRLSCAASGLTFPNYGMSWFRQAPGKGLEFVCAIYWSGGTVYYAESVKGRFTCSRDNAKNTLYLQMSSLRAEDTAVYYCAVTIRGAATQTWKYDYWGQGTLVTVKPGGGG

SEQ ID NO: 93 - protein, 256 aa, synthetic construct
  REGION 1..256: note = chemically synthesized
EVQLLESGGGEVQPGGSLRLSCAASGLTFPNYGMSWFRQAPGKGLEFVSAIYWSGGTVYYAESVKGRFTISRDNAKNTLYLQMSSLRAEDTAVYYCAVTIRGAATQTWKYDYWGQGTLVTVKPGGSGGSEVQLLESGGGEVQPGGSLRLSCAASGLTFPNYGMSWFRQAPGKGLEFVSAIYWSGGTVYYAESVKGRFTISRDNAKNTLYLQMSSLRAEDTAVYYCAVTIRGAATQTWKYDYWGQGTLVTVKPGGGG

SEQ ID NO: 94 - protein, 267 aa, synthetic construct
  REGION 1..267: note = chemically synthesized
EVQLLESGGGLVQPGGSLRLSCAASGSTFSSLDMGWFRQAPGKGREFVSAISRSGDNIYYADSVKGRFTISRDNSKNTLYLQMNSLRAEDTAVYYCAVDSQPTYSGGVYYPRYGMDVWGQGTLVTVSGSGGGGSEVQLLESGGGLVQPGGSLRLSCAASGSTFSSLDMGWFRQAPGKGREFVSAISRSGDNIYYADSVKGRFTISRDNSKNTLYLQMNSLRAEDTAVYYCAVDSQPTYSGGVYYPRYGMDVWGQGTLVTVSSAGGGG

SEQ ID NO: 95 - protein, 263 aa, synthetic construct
  REGION 1..263: note = chemically synthesized
EVQLLESGGGLVQPGGSLRLSCAASGLTFPNYGMSWFRQAPGKGLEFVSAIYWSGGTVYYADSVKGRFTISRDNSKNTLYLQMNSLRAEDTAVYYCAVTIRGAATQTWKYDYWGQGTLVTVSSGGGSGGSGGGGSEVQLLESGGGLVQPGGSLRLSCAASGLTFPNYGMSWFRQAPGKGLEFVSAIYWSGGTVYYADSVKGRFTISRDNSKNTLYLQMNSLRAEDTAVYYCAVTIRGAATQTWKYDYWGQGTLVTVSSAGGGG

SEQ ID NO: 96 - protein, 266 aa, synthetic construct
  REGION 1..266: note = chemically synthesized
EVQLLESGGGLVQPGGSLRLSCAASGLTFPNYGMSWFRQAPGKGLEFVSAIYWSGGTVYYADSVKGRFTISRDNSKNTLYLQMNSLRAEDTAVYYCAVTIRGAATQTWKYDYWGQGTLVTVSSGGGGSGGGGSGGGGSEVQLLESGGGLVQPGGSLRLSCAASGLTFPNYGMSWFRQAPGKGLEFVSAIYWSGGTVYYADSVKGRFTISRDNSKNTLYLQMNSLRAEDTAVYYCAVTIRGAATQTWKYDYWGQGTLVTVSSAGGGG

SEQ ID NO: 97 - protein, 267 aa, synthetic construct; identical to SEQ ID NO: 94
  REGION 1..267: note = chemically synthesized

SEQ ID NO: 98 - protein, 270 aa, synthetic construct
  REGION 1..270: note = chemically synthesized
EVQLLESGGGLVQPGGSLRLSCAASGSTFSSLDMGWFRQAPGKGREFVSAISRSGDNIYYADSVKGRFTISRDNSKNTLYLQMNSLRAEDTAVYYCAVDSQPTYSGGVYYPRYGMDVWGQGTLVTVSSGGGSGGGGSEVQLLESGGGLVQPGGSLRLSCAASGSTFSSLDMGWFRQAPGKGREFVSAISRSGDNIYYADSVKGRFTISRDNSKNTLYLQMNSLRAEDTAVYYCAVDSQPTYSGGVYYPRYGMDVWGQGTLVTVSSAGGGG

SEQ ID NO: 99 - protein, 273 aa, synthetic construct
  REGION 1..273: note = chemically synthesized
EVQLLESGGGLVQPGGSLRLSCAASGSTFSSLDMGWFRQAPGKGREFVSAISRSGDNIYYADSVKGRFTISRDNSKNTLYLQMNSLRAEDTAVYYCAVDSQPTYSGGVYYPRYGMDVWGQGTLVTVSSGGGSGGSGGGGSEVQLLESGGGLVQPGGSLRLSCAASGSTFSSLDMGWFRQAPGKGREFVSAISRSGDNIYYADSVKGRFTISRDNSKNTLYLQMNSLRAEDTAVYYCAVDSQPTYSGGVYYPRYGMDVWGQGTLVTVSSAGGGG

SEQ ID NO: 100 - protein, 276 aa, synthetic construct
  REGION 1..276: note = chemically synthesized
EVQLLESGGGLVQPGGSLRLSCAASGSTFSSLDMSWFRQAPGKGLEFVSAISRSGDNIYYADSVKGRFTISRDNSKNTLYLQMNSLRAEDTAVYYCAVDSQPTYSGGVYYPRYGMDVWGQGTLVTVSSGGGGSGGGGSGGGGSEVQLLESGGGLVQPGGSLRLSCAASGSTFSSLDMSWFRQAPGKGLEFVSAISRSGDNIYYADSVKGRFTISRDNSKNTLYLQMNSLRAEDTAVYYCAVDSQPTYSGGVYYPRYGMDVWGQGTLVTVSSAGGGG

SEQ ID NO: 101 - protein, 259 aa, synthetic construct
  REGION 1..259: note = chemically synthesized
EVQLLESGGGLVQPGGSLRLSCAASGRAFSNYAMSWFRQAPGKGLEFVSAINWNGENRYYADSVKGRFTISRDNSKNTLYLQMNSLRAEDTAVYYCAAALSFRLGGEPYGDAYWGQGTLVTVSGSGGGGSEVQLLESGGGLVQPGGSLRLSCAASGRAFSNYAMSWFRQAPGKGLEFVSAINWNGENRYYADSVKGRFTISRDNSKNTLYLQMNSLRAEDTAVYYCAAALSFRLGGEPYGDAYWGQGTLVTVSSAGGGG

SEQ ID NO: 102 - protein, 265 aa, synthetic construct
  REGION 1..265: note = chemically synthesized
EVQLLESGGGLVQPGGSLRLSCAASGRAFSNYAMSWFRQAPGKGLEFVSAINWNGENRYYADSVKGRFTISRDNSKNTLYLQMNSLRAEDTAVYYCAAALSFRLGGEPYGDAYWGQGTLVTVSSGGGSGGSGGGGSEVQLLESGGGLVQPGGSLRLSCAASGRAFSNYAMSWFRQAPGKGLEFVSAINWNGENRYYADSVKGRFTISRDNSKNTLYLQMNSLRAEDTAVYYCAAALSFRLGGEPYGDAYWGQGTLVTVSSAGGGG

SEQ ID NO: 103 - protein, 268 aa, synthetic construct
  REGION 1..268: note = chemically synthesized
EVQLLESGGGLVQPGGSLRLSCAASGRAFSNYAMSWFRQAPGKGLEFVSAINWNGENRYYADSVKGRFTISRDNSKNTLYLQMNSLRAEDTAVYYCAAALSFRLGGEPYGDAYWGQGTLVTVSSGGGGSGGGGSGGGGSEVQLLESGGGLVQPGGSLRLSCAASGRAFSNYAMSWFRQAPGKGLEFVSAINWNGENRYYADSVKGRFTISRDNSKNTLYLQMNSLRAEDTAVYYCAAALSFRLGGEPYGDAYWGQGTLVTVSSAGGGG

SEQ ID NO: 104 - protein, 261 aa, synthetic construct
  REGION 1..261: note = chemically synthesized
EVQLLESGGGLVQPGGSLRLSCAASGRSISNYAMGWFRQAPGKEREFVSASVWNNGGNYYADSVKGRFTISRDNSKNTLYLQMNSLRAEDTAVYYCVVARTPETPITSARGANYWGQGTLVTVSGSGGGGSEVQLLESGGGLVQPGGSLRLSCAASGRSISNYAMGWFRQAPGKEREFVSASVWNNGGNYYADSVKGRFTISRDNSKNTLYLQMNSLRAEDTAVYYCVVARTPETPITSARGANYWGQGTLVTVSSAGGGG

SEQ ID NO: 105 - protein, 261 aa, synthetic construct
  REGION 1..261: note = chemically synthesized
EVQLLESGGGKVQPGGSLRLSCAASGRSISNYAMSWFRQAPGKGLEFVSASVWNNGGNYYADSVKGRFTISRDNSKNTLYLQMNSLRAEDTAVYYCVVARTPETPITSARGANYWGQGTLVTVSGSGGGGSEVQLLESGGGLVQPGGSLRLSCAASGRSISNYAMSWFRQAPGKGLEFVSASVWNNGGNYYADSVKGRFTISRDNSKNTLYLQMNSLRAEDTAVYYCVVARTPETPITSARGANYWGQGTLVTVSSAGGGG

SEQ ID NO: 106 - protein, 264 aa, synthetic construct
  REGION 1..264: note = chemically synthesized
EVQLLESGGGLVQPGGSLRLSCAASGRSISNYAMSWFRQAPGKGLEFVSASVWNNGGNYYADSVKGRFTISRDNSKNTLYLQMNSLRAEDTAVYYCVVARTPETPITSARGANYWGQGTLVTVSSGGGSGGGGSEVQLLESGGGLVQPGGSLRLSCAASGRSISNYAMSWFRQAPGKGLEFVSASVWNNGGNYYADSVKGRFTISRDNSKNTLYLQMNSLRAEDTAVYYCVVARTPETPITSARGANYWGQGTLVTVSSAGGGG

SEQ ID NO: 107 - protein, 267 aa, synthetic construct
  REGION 1..267: note = chemically synthesized
EVQLLESGGGLVQPGGSLRLSCAASGRSISNYAMSWFRQAPGKGLEFVSASVWNNGGNYYADSVKGRFTISRDNSKNTLYLQMNSLRAEDTAVYYCVVARTPETPITSARGANYWGQGTLVTVSSGGGSGGSGGGGSEVQLLESGGGLVQPGGSLRLSCAASGRSISNYAMSWFRQAPGKGLEFVSASVWNNGGNYYADSVKGRFTISRDNSKNTLYLQMNSLRAEDTAVYYCVVARTPETPITSARGANYWGQGTLVTVSSAGGGG

SEQ ID NO: 108 - protein, 261 aa, synthetic construct; identical to SEQ ID NO: 105
  REGION 1..261: note = chemically synthesized

SEQ ID NO: 109 - protein, 263 aa, synthetic construct
  REGION 1..263: note = chemically synthesized
EVQLLESGGGLVQPGGSLRLSCAASVSTFGTSPVGWFRQAPGKEREFVSAIRWDGVGAYYADSVKGRFTISRDNSKNTLYLQMNSLRAEDTAVYYCALPRRGDSELPSTVKEYGYWGQGTLVTVSGSGGGGSEVQLLESGGGLVQPGGSLRLSCAASVSTFGTSPVGWFRQAPGKEREFVSAIRWDGVGAYYADSVKGRFTISRDNSKNTLYLQMNSLRAEDTAVYYCALPRRGDSELPSTVKEYGYWGQGTLVTVSSAGGGG

SEQ ID NO: 110 - protein, 272 aa, synthetic construct
  REGION 1..272: note = chemically synthesized
EVQLLESGGGLVQPGGSLRLSCAASVSTFGTSPVGWFRQAPGKEREFVSAIRWDGVGAYYADSVKGRFTISRDNSKNTLYLQMNSLRAEDTAVYYCALPRRGDSELPSTVKEYGYWGQGTLVTVSSGGGGSGGGGSGGGGSEVQLLESGGGLVQPGGSLRLSCAASVSTFGTSPVGWFRQAPGKEREFVSAIRWDGVGAYYADSVKGRFTISRDNSKNTLYLQMNSLRAEDTAVYYCALPRRGDSELPSTVKEYGYWGQGTLVTVSSAGGGG

SEQ ID NO: 111 - protein, 265 aa, synthetic construct
  REGION 1..265: note = chemically synthesized
EVQLLESGGGEVQPGGSLRLSCAASGLTFPNYGMGWFRQAPGKEREFVSAIYWSGGTVFYAESVKGRFTISRDNAKNTVYLQMSSLRAEDTAVYYCAVTIRGAATQTWKYDYWGQGTLVTVKPGGSGGSEVQLLESGGGEVQPGGSLRLSCAASGLTFPNYGMGWFRQAPGKEREFVSAIYWSGGTVFYAESVKGRFTISRDNAKNTVYLQMSSLRAEDTAVYYCAVTIRGAATQTWKYDYWGQGTLVTVKPGGGGDKTHTCPPC

SEQ ID NO: 112 - protein, 265 aa, synthetic construct
  REGION 1..265: note = chemically synthesized
EVQLLESGGGEVQPGGSLRLSCAASGLTFPNYGMGWFRQAPGKEREFLAVIYWSGGTVFYAESVKGRFTISRDNAKNTVYLQMSSLRAEDTAVYYCAVTIRGAATQTWKYDYWGQGTLVTVKPGGSGGSEVQLLESGGGEVQPGGSLRLSCAASGLTFPNYGMGWFRQAPGKEREFLAVIYWSGGTVFYAESVKGRFTISRDNAKNTVYLQMSSLRAEDTAVYYCAVTIRGAATQTWKYDYWGQGTLVTVKPGGGGDKTHTCPPC

SEQ ID NO: 113 - protein, 265 aa, synthetic construct
  REGION 1..265: note = chemically synthesized
EVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGGSGGSEVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGGGGDKTHTCPPC

SEQ ID NO: 114 - protein, 265 aa, synthetic construct
  REGION 1..265: note = chemically synthesized
EVQLLESGGGEVQPGGSLRLSCAASGLTFPNYGMGWFRQAPGKEREFLAVIYWSGGTVYYAESVKGRFTISRDNAKNTLYLQMSSLRAEDTAVYYCAVTIRGAATQTWKYDYWGQGTLVTVKPGGSGGSEVQLLESGGGEVQPGGSLRLSCAASGLTFPNYGMGWFRQAPGKEREFLAVIYWSGGTVYYAESVKGRFTISRDNAKNTLYLQMSSLRAEDTAVYYCAVTIRGAATQTWKYDYWGQGTLVTVKPGGGGDKTHTCPPC

SEQ ID NO: 115 - protein, 265 aa, synthetic construct
  REGION 1..265: note = chemically synthesized
EVQLLESGGGEVQPGGSLRLSCAASGLTFPNYGMGWFRQAPGKEREFVSAIYWSGGTVYYAESVKGRFTISRDNAKNTVYLQMSSLRAEDTAVYYCAVTIRGAATQTWKYDYWGQGTLVTVKPGGSGGSEVQLLESGGGEVQPGGSLRLSCAASGLTFPNYGMGWFRQAPGKEREFVSAIYWSGGTVYYAESVKGRFTISRDNAKNTVYLQMSSLRAEDTAVYYCAVTIRGAATQTWKYDYWGQGTLVTVKPGGGGDKTHTCPPC

SEQ ID NO: 116 - protein, 265 aa, synthetic construct
  REGION 1..265: note = chemically synthesized
EVQLLESGGGEVQPGGSLRLSCAASGLTFPNYGMGWFRQAPGKEREFLAVIYWSGGTVYYAESVKGRFTISRDNAKNTVYLQMSSLRAEDTAVYYCAVTIRGAATQTWKYDYWGQGTLVTVKPGGSGGSEVQLLESGGGEVQPGGSLRLSCAASGLTFPNYGMGWFRQAPGKEREFLAVIYWSGGTVYYAESVKGRFTISRDNAKNTVYLQMSSLRAEDTAVYYCAVTIRGAATQTWKYDYWGQGTLVTVKPGGGGDKTHTCPPC

SEQ ID NO: 117 - protein, 267 aa, synthetic construct
  REGION 1..267: note = chemically synthesized
EVQLLESGGGEVQPGGSLRLSCAASGRTVSNYAMGWFRQAPGKDREFVSALNWGGDTTYYAESVKGRFTISRDNAKNTLYLQMSSLRAEDTAVYYCAAAQSFRRGGAPYGDNYWGQGTLVTVKPGGSGGSEVQLLESGGGEVQPGGSLRLSCAASGRTVSNYAMGWFRQAPGKDREFVSALNWGGDTTYYAESVKGRFTISRDNAKNTLYLQMSSLRAEDTAVYYCAAAQSFRRGGAPYGDNYWGQGTLVTVKPGGGGDKTHTCPPC

SEQ ID NO: 118 - protein, 270 aa, synthetic construct
  REGION 1..270: note = chemically synthesized
EVQLLESGGGEVQPGGSLRLSCAASGRTVSNYAMGWFRQAPGKDREFVSALNWGGDTTYYAESVKGRFTISRDNAKNTLYLQMSSLRAEDTAVYYCAAAQSFRRGGAPYGDNYWGQGTLVTVKPGGSGGSGGSEVQLLESGGGEVQPGGSLRLSCAASGRTVSNYAMGWFRQAPGKDREFVSALNWGGDTTYYAESVKGRFTISRDNAKNTLYLQMSSLRAEDTAVYYCAAAQSFRRGGAPYGDNYWGQGTLVTVKPGGGGDKTHTCPPC

SEQ ID NO: 119 - protein, 410 aa, synthetic construct
  REGION 1..410: note = chemically synthesized
EVQLLESGGGLVQPGGSLRLSCAASGRSISNYAMSWFRQAPGKGLEFVSASVWNNGGNYYADSVKGRFTISRDNSKNTLYLQMNSLRAEDTAVYYCVVARTPETPITSARGANYWGQGTLVTVSSGGGGSGGGGSGGGGSEVQLLESGGGLVQPGGSLRLSCAASGRSISNYAMSWFRQAPGKGLEFVSASVWNNGGNYYADSVKGRFTISRDNSKNTLYLQMNSLRAEDTAVYYCVVARTPETPITSARGANYWGQGTLVTVSSGGGGSGGGGSGGGGSEVQLLESGGGLVQPGGSLRLSCAASGRSISNYAMSWFRQAPGKGLEFVSASVWNNGGNYYADSVKGRFTISRDNSKNTLYLQMNSLRAEDTAVYYCVVARTPETPITSARGANYWGQGTLVTVSSAGGGG

SEQ ID NO: 120 - protein, 394 aa, synthetic construct
  REGION 1..394: note = chemically synthesized
EVQLLESGGGEVQPGGSLRLSCAASVSTFGTSPVGWFRQAPGKEREFVCAIRWEGVGAYYAESVKGRFTCSRDNAKNTLYLQMSSLRAEDTAVYYCALPRRGDSELPSTVKEYGYWGQGTLVTVKPGGSGGSEVQLLESGGGEVQPGGSLRLSCAASVSTFGTSPVGWFRQAPGKEREFVCAIRWEGVGAYYAESVKGRFTCSRDNAKNTLYLQMSSLRAEDTAVYYCALPRRGDSELPSTVKEYGYWGQGTLVTVKPGGSGGSEVQLLESGGGEVQPGGSLRLSCAASVSTFGTSPVGWFRQAPGKEREFVCAIRWEGVGAYYAESVKGRFTCSRDNAKNTLYLQMSSLRAEDTAVYYCALPRRGDSELPSTVKEYGYWGQGTLVTVKPGGGG

SEQ ID NO: 121 - protein, 394 aa, synthetic construct
  REGION 1..394: note = chemically synthesized
EVQLLESGGGEVQPGGSLRLSCAASVSTFGTSPVGWFRQAPGKEREFVSAIRWEGVGAYYAESVKGRFTISRDNAKNTLYLQMSSLRAEDTAVYYCALPRRGDSELPSTVKEYGYWGQGTLVTVKPGGSGGSEVQLLESGGGEVQPGGSLRLSCAASVSTFGTSPVGWFRQAPGKEREFVSAIRWEGVGAYYAESVKGRFTISRDNAKNTLYLQMSSLRAEDTAVYYCALPRRGDSELPSTVKEYGYWGQGTLVTVKPGGSGGSEVQLLESGGGEVQPGGSLRLSCAASVSTFGTSPVGWFRQAPGKEREFVSAIRWEGVGAYYAESVKGRFTISRDNAKNTLYLQMSSLRAEDTAVYYCALPRRGDSELPSTVKEYGYWGQGTLVTVKPGGGG

SEQ ID NO: 122 - protein, 400 aa, synthetic construct
  REGION 1..400: note = chemically synthesized
EVQLLESGGGEVQPGGSLRLSCAASGSTFSSLDMGWFRQAPGKGREFVCAISRSGDNIYYAESVKGRFTCSRDNAKNTLYLQMSSLRAEDTAVYYCAVESQPTYSGGVYYPRYGMDVWGQGTLVTVKPGGSGGSEVQLLESGGGEVQPGGSLRLSCAASGSTFSSLDMGWFRQAPGKGREFVCAISRSGDNIYYAESVKGRFTCSRDNAKNTLYLQMSSLRAEDTAVYYCAVESQPTYSGGVYYPRYGMDVWGQGTLVTVKPGGSGGSEVQLLESGGGEVQPGGSLRLSCAASGSTFSSLDMGWFRQAPGKGREFVCAISRSGDNIYYAESVKGRFTCSRDNAKNTLYLQMSSLRAEDTAVYYCAVESQPTYSGGVYYPRYGMDVWGQGTLVTVKPGGGG

SEQ ID NO: 123 - protein, 400 aa, synthetic construct
  REGION 1..400: note = chemically synthesized
EVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVESQPTYSGGVYYPRYGMDVWGQGTLVTVKPGGSGGSEVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVESQPTYSGGVYYPRYGMDVWGQGTLVTVKPGGSGGSEVQLLESGGGEVQPGGSLRLSCAASGSTFSSLDMGWFRQAPGKGREFVSAISRSGDNIYYAESVKGRFTISRDNAKNTLYLQMSSLRAEDTAVYYCAVESQPTYSGGVYYPRYGMDVWGQGTLVTVKPGGGG

SEQ ID NO: 124 - protein, 413 aa, synthetic construct
  REGION 1..413: note = chemically synthesized
QVQLVQSGGGLVQAGGSLTLSCAASVSTFGTSPVGWFRQAPGKEREFVSAIRWDGVGAYYADSVRGRFKNSKDNAKRTAYLQMNRLKPEDTAVYYCALPRRGDSELPSTVKEYGYWGQGTQVTVSSGGGGSGGGGSGGGGSQVQLVQSGGGLVQAGGSLTLSCAASVSTFGTSPVGWFRQAPGKEREFVSAIRWDGVGAYYADSVRGRFKNSKDNAKRTAYLQMNRLKPEDTAVYYCALPRRGDSELPSTVKEYGYWGQGTQVTVSSGGGGSGGGGSGGGGSQVQLVQSGGGLVQAGGSLTLSCAASVSTFGTSPVGWFRQAPGKEREFVSAIRWDGVGAYYADSVRGRFKNSKDNAKRTAYLQMNRLKPEDTAVYYCALPRRGDSELPSTVKEYGYWGQGTQVTVSSAGGGG

SEQ ID NO: 125 - protein, 593 aa, synthetic construct
  REGION 1..593: note = chemically synthesized
EVQLLESGGGLVQPGGSLRLSCAASGTFDKINNMGWYRQAPGKQRDLVAQITPGGITDYADSVKGRFTISRDNSKNTLYLQMNSLRPEDTAVYYCNAEILKRAYIDVYVNYWGQGTLVTVSSGGGGSGGGGSGGGGSGGGGSGGGGSGGGGSGGGGSEVQLLESGGGLVQPGGSLRLSCAASGTFDKINNMGWYRQAPGKQRDLVAQITPGGITDYADSVKGRFTISRDNSKNTLYLQMNSLRPEDTAVYYCNAEILKRAYIDVYVNYWGQGTLVTVSSGGGGSGGGGSGGGGSGGGGSGGGGSGGGGSGGGGSEVQLLESGGGLVQPGGSLRLSCAASGTFDKINNMGWYRQAPGKQRDLVAQITPGGITDYADSVKGRFTISRDNSKNTLYLQMNSLRPEDTAVYYCNAEILKRAYIDVYVNYWGQGTLVTVSSGGGGSGGGGSGGGGSGGGGSGGGGSGGGGSGGGGSEVQLLESGGGLVQPGGSLRLSCAASGTFDKINNMGWYRQAPGKQRDLVAQITPGGITDYADSVKGRFTISRDNSKNTLYLQMNSLRPEDTAVYYCNAEILKRAYIDVYVNYWGQGTLVTVSS

SEQ ID NO: 126 - protein, 595 aa, synthetic construct
  REGION 1..595: note = chemically synthesized
EVQLLESGGGEVQPGGSLRLSCAASGTFDKINNMGWYRQAPGKQRDLVAQITPGGITDYADSVKGRFTISRDNSKNTLYLQMNSLRPEDTAVYYCNAEILKRAYIDVYVNYWGQGTLVTVKPGGGGSGGGGSGGGGSGGGGSGGGGSGGGGSGGGGSEVQLLESGGGEVQPGGSLRLSCAASGTFDKINNMGWYRQAPGKQRDLVAQITPGGITDYADSVKGRFTISRDNSKNTLYLQMNSLRPEDTAVYYCNAEILKRAYIDVYVNYWGQGTLVTVKPGGGGSGGGGSGGGGSGGGGSGGGGSGGGGSGGGGSEVQLLESGGGEVQPGGSLRLSCAASGTFDKINNMGWYRQAPGKQRDLVAQITPGGITDYADSVKGRFTISRDNSKNTLYLQMNSLRPEDTAVYYCNAEILKRAYIDVYVNYWGQGTLVTVKPGGGGSGGGGSGGGGSGGGGSGGGGSGGGGSGGGGSEVQLLESGGGEVQPGGSLRLSCAASGTFDKINNMGWYRQAPGKQRDLVAQITPGGITDYADSVKGRFTISRDNSKNTLYLQMNSLRPEDTAVYYCNAEILKRAYIDVYVNYWGQGTLVTVKPGG

SEQ ID NO: 127 - protein, 218 aa, synthetic construct
  REGION 1..218: note = chemically synthesized
PAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 128 - protein, 10 aa, synthetic construct
  REGION 1..10: note = chemically synthesized
SGLTFPNYGM

SEQ ID NO: 129 - protein, 10 aa, synthetic construct
  REGION 1..10: note = chemically synthesized
VIYWSGGTVF

SEQ ID NO: 130 - protein, 17 aa, synthetic construct
  REGION 1..17: note = chemically synthesized
AVTIRGAATQTWKYDYW

SEQ ID NO: 131 - protein, 10 aa, synthetic construct
  REGION 1..10: note = chemically synthesized
AIYWSGGTVY

SEQ ID NO: 132 - protein, 10 aa, synthetic construct
  REGION 1..10: note = chemically synthesized
AIYWSGGTVF

SEQ ID NO: 133 - protein, 10 aa, synthetic construct
  REGION 1..10: note = chemically synthesized
VIYWSGGTVY

SEQ ID NO: 134 - protein, 10 aa, synthetic construct
  REGION 1..10: note = chemically synthesized
SGRTVSNYAM

SEQ ID NO: 135 - protein, 10 aa, synthetic construct
  REGION 1..10: note = chemically synthesized
ALNWGGDTTS

SEQ ID NO: 136 - protein, 18 aa, synthetic construct
  REGION 1..18: note = chemically synthesized
AAAQSFRRGGAPYGDNYW

SEQ ID NO: 137 - protein, 10 aa, synthetic construct
  REGION 1..10: note = chemically synthesized
ALNWGGDTTY

SEQ ID NO: 138 - protein, 12 aa, synthetic construct
  REGION 1..12: note = chemically synthesized
SGRALTGYHMAW

SEQ ID NO: 139 - protein, 10 aa, synthetic construct
  REGION 1..10: note = chemically synthesized
YGIWDRAGAA

SEQ ID NO: 140 - protein, 17 aa, synthetic construct
  REGION 1..17: note = chemically synthesized
ASMAVRTYYSPRSYDSW

SEQ ID NO: 141 - protein, 12 aa, synthetic construct
  REGION 1..12: note = chemically synthesized
SGRALTGYHMSW

SEQ ID NO: 142 - protein, 12 aa, synthetic construct
  REGION 1..12: note = chemically synthesized
SGSTFSSLDMGW

SEQ ID NO: 143 - protein, 10 aa, synthetic construct
  REGION 1..10: note = chemically synthesized
AISRSGDNIY

SEQ ID NO: 144 - protein, 22 aa, synthetic construct
  REGION 1..22: note = chemically synthesized
AVDSQPTYSGGVYYPRYGMDVW

SEQ ID NO: 145 - protein, 22 aa, synthetic construct
  REGION 1..22: note = chemically synthesized
AVDTQPTYSGGVYYPRYGMDVW

SEQ ID NO: 146 - protein, 22 aa, synthetic construct
  REGION 1..22: note = chemically synthesized
AVDAQPTYSGGVYYPRYGMDVW

SEQ ID NO: 147 - protein, 22 aa, synthetic construct
  REGION 1..22: note = chemically synthesized
AVESQPTYSGGVYYPRYGMDVW

SEQ ID NO: 148 - protein, 22 aa, synthetic construct
  REGION 1..22: note = chemically synthesized
AVDSQPTYSGGVYYPRYGYDVW

SEQ ID NO: 149 - protein, 22 aa, synthetic construct
  REGION 1..22: note = chemically synthesized
AVDSQPTYSGGVYYPRYGLDVW

SEQ ID NO: 150 - protein, 22 aa, synthetic construct
  REGION 1..22: note = chemically synthesized
AVDAQPTYSGGVYYPRYGADVW

SEQ ID NO: 151 - protein, 22 aa, synthetic construct
  REGION 1..22: note = chemically synthesized
AVDAQPTYSGGVYYPRYGHDVW

SEQ ID NO: 152 - protein, 22 aa, synthetic construct
  REGION 1..22: note = chemically synthesized
AVDAQPTYSGGVYYPRYGNDVW

SEQ ID NO: 153 - protein, 22 aa, synthetic construct
  REGION 1..22: note = chemically synthesized
AVDAQPTYSGGVYYPRYGPDVW

SEQ ID NO: 154 - protein, 22 aa, synthetic construct
  REGION 1..22: note = chemically synthesized
AVDAQPTYSGGVYYPRYGQDVW

SEQ ID NO: 155 - protein, 22 aa, synthetic construct
  REGION 1..22: note = chemically synthesized
AVDAQPTYSGGVYYPRYGRDVW

SEQ ID NO: 156 - protein, 22 aa, synthetic construct
  REGION 1..22: note = chemically synthesized
AVDAQPTYSGGVYYPRYGSDVW

SEQ ID NO: 157 - protein, 22 aa, synthetic construct
  REGION 1..22: note = chemically synthesized
AVDAQPTYSGGVYYPRYGTDVW

SEQ ID NO: 158 - protein, 22 aa, synthetic construct
  REGION 1..22: note = chemically synthesized
AVDAQPTYSGGVYYPRYGVDVW

SEQ ID NO: 159 - protein, 12 aa, synthetic construct
  REGION 1..12: note = chemically synthesized
SGRAFSNYALGW

SEQ ID NO: 160 - protein, 10 aa, synthetic construct
  REGION 1..10: note = chemically synthesized
AINWNGENRY

SEQ ID NO: 161 - protein, 18 aa, synthetic construct
  REGION 1..18: note = chemically synthesized
AAALSFRLGGEPYGDAYW

SEQ ID NO: 162 - protein, 12 aa, synthetic construct
  REGION 1..12: note = chemically synthesized
SGRAFSNYAMSW

SEQ ID NO: 163 - protein, 10 aa, synthetic construct
  REGION 1..10: note = chemically synthesized
SGSIFTNNAM

SEQ ID NO: 164 - protein, 10 aa, synthetic construct
  REGION 1..10: note = chemically synthesized
QITMGGGITN

SEQ ID NO: 165 - protein, 16 aa, synthetic construct
  REGION 1..16: note = chemically synthesized
NAEVKSADWGAYANYW

SEQ ID NO: 166 - protein, 10 aa, synthetic construct
  REGION 1..10: note = chemically synthesized
AITMGGGITY

SEQ ID NO: 167 - protein, 10 aa, synthetic construct
  REGION 1..10: note = chemically synthesized
QITMGGGITY

SEQ ID NO: 168 - protein, 10 aa, synthetic construct
  REGION 1..10: note = chemically synthesized
SGRSISNYAM

SEQ ID NO: 169 - protein, 10 aa, synthetic construct
  REGION 1..10: note = chemically synthesized
ASVWNNGGNY

SEQ ID NO: 170 - protein, 19 aa, synthetic construct
  REGION 1..19: note = chemically synthesized
VVARTPETPITSARGANYW

SEQ ID NO: 171 - protein, 10 aa, synthetic construct
  REGION 1..10: note = chemically synthesized
ASVWNQGGNY

SEQ ID NO: 172 - protein, 10 aa, synthetic construct
  REGION 1..10: note = chemically synthesized
ASVWNNAGNY

SEQ ID NO: 173 - protein, 10 aa, synthetic construct
  REGION 1..10: note = chemically synthesized
SGRAASDYAV

SEQ ID NO: 174 - protein, 10 aa, synthetic construct
  REGION 1..10: note = chemically synthesized
ACNWSGEDTV

SEQ ID NO: 175 - protein, 21 aa, synthetic construct
  REGION 1..21: note = chemically synthesized
AAAPSFSRSVLDGNLSQIDYW

SEQ ID NO: 176 - protein, 10 aa, synthetic construct
  REGION 1..10: note = chemically synthesized
SGRAASDYAM

SEQ ID NO: 177 - protein, 9 aa, synthetic construct
  REGION 1..9: note = chemically synthesized
INWGGEDTV

SEQ ID NO: 178 - protein, 10 aa, synthetic construct
  REGION 1..10: note = chemically synthesized
SGRTFTNYAM

SEQ ID NO: 179 - protein, 10 aa, synthetic construct
  REGION 1..10: note = chemically synthesized
AINWSGDSTY

SEQ ID NO: 180 - protein, 18 aa, synthetic construct
  REGION 1..18: note = chemically synthesized
ASAESFSRGGLPYGMNYW

SEQ ID NO: 181 - protein, 10 aa, synthetic construct
  REGION 1..10: note = chemically synthesized
SVSTFGTSPV

SEQ ID NO: 182 - protein, 10 aa, synthetic construct
  REGION 1..10: note = chemically synthesized
AIRWDGVGAY

SEQ ID NO: 183 - protein, 20 aa, synthetic construct
  REGION 1..20: note = chemically synthesized
ALPRRGDSELPSTVKEYGYW

SEQ ID NO: 184 - protein, 10 aa, synthetic construct
  REGION 1..10: note = chemically synthesized
AIRWEGVGAY

SEQ ID NO: 185 - protein, 10 aa, synthetic construct
  REGION 1..10: note = chemically synthesized
AIRWDAVGAY

SEQ ID NO: 186 - protein, 20 aa, synthetic construct
  REGION 1..20: note = chemically synthesized
ALPRRGESELPSTVKEYGYW

SEQ ID NO: 187 - protein, 20 aa, synthetic construct
  REGION 1..20: note = chemically synthesized
ALPRRGDAELPSTVKEYGYW

SEQ ID NO: 188 - protein, 10 aa, synthetic construct
  REGION 1..10: note = chemically synthesized
SGRTLSAYLM

SEQ ID NO: 189 - protein, 10 aa, synthetic construct
  REGION 1..10: note = chemically synthesized
RIRWNEGDTY

SEQ ID NO: 190 - protein, 15 aa, synthetic construct
  REGION 1..15: note = chemically synthesized
AARSIFNPSDQYVYW